(19) United States
(12) Patent Application Publication
(10) Pub. No.: US 2010/0168011 A1
(43) Pub. Date: Jul. 1, 2010

(54) Pharmaceutical Formulations and Methods for Making the Same

(75) Inventors: Robert N. Jennings, JR., San Diego, CA (US); Scott H. Coleman, San Diego, CA (US)

Correspondence Address: Intellectual Property Department;Amylin Pharmaceuticals, Inc., 9360 Towne Centre Drive, San Diego, CA, 92121, US

(73) Assignee: Amylin Pharmaceuticals, Inc., San Diego, CA (US)

(21) Appl. No.: 12/519,114

(22) PCT Filed: Dec. 11, 2007
(86) PCT No.: PCT/US2007/025382

§ 371 Date: Jun. 12, 2009

Related U.S. Application Data
(60) Provisional application No. 60/874,647, filed on Dec. 12, 2006.

(51) Int. Cl. A61K 38/16; A61P 3/10

(52) U.S. Cl. 514/12

(57) ABSTRACT

In certain embodiments, the invention relates to pre-lyophilization formulations, lyophilized compositions, reconstituted formulations, kits comprising the same, and methods for preparing, storing and using the same.

### BACKGROUND

Most peptides and proteins lose activity when stored in aqueous solutions for any extended period of time. Even when refrigerated, long-term stability of proteins and peptides can be a problem. Lyophilization, a freeze-drying process that removes 95% or more of the water from a formulation, has been employed to stabilize pharmaceutical compositions containing peptides or proteins.

Lyophilization generally involves a freezing stage in which a formulation is solidified, a primary drying stage in which ice is removed by sublimation under vacuum or reduced pressure, and a secondary drying stage in which residual water is removed. The desired output of the process is a solid composition which can be stored for extended periods of time and readily reconstituted to yield a biologically active peptide or protein.

During the freezing stage, ice nucleates and grows within the formulation solution. Freezing can cause damage to proteins/peptides and/or aggregation as the concentrations of proteins/peptides and buffer salts increase. Additionally, the concentration of buffer salts can lead to their precipitation. See, e.g., Eckhardt B M, et al. Pharm. Res. 1991; 8:1360-1364 and Van den Berg L and Rose, D Arch. Biochem. Biophys. 1959; 81:319-329. Events that occur during the freezing stage also will impact the primary and secondary drying stages. For example, ice crystal morphology will affect the porosity of the solid or “cake” that forms as ice sublimes during the primary drying stage and the ultimate consistency of the cake after the secondary drying stage.

Cryoprotectants and lyoprotectants are often added to formulations prior to lyophilization. Cryoprotectants are provided to stabilize proteins during the freezing process and may also provide protection during primary and secondary drying, as well as during long-term storage. Examples of cryoprotectants include dextran, polyethylene glycol, sugars, such as sucrose, glucose, trehalose, and lactose; surfactants such as polysorbates; and free amino acids such as glycine, arginine, and serine. Lyoprotectants are added to provide stability during primary and secondary drying processes. Examples of lyoprotectants include polyols and sugars such as sucrose and trehalose. See, e.g., Carpenter J F, et al. Arch. Biochem. Biophys. 1986; 250:505-512; Carpenter J F and Crowe J H Cryobiology 1988; 25:459-470; Carpenter J F and Crowe J H Cryobiology 1988; 25:244-255; Carpenter J F and Crowe J H Biochem 1989; 28:3916-3922; Carpenter J F, et al., J. Diary Sci. 1990; 73:3627-3636; Carpenter J F, et al. Achives of Biochemistry and Biophysics. 1993; 303:456-464; and Prestrelski S J, et al. Achives of Biochemistry and Biophysics. 1993; 303, 465-473.

High solid concentrations may be required for certain pharmaceutical formulations. U.S. Patent Publication 20040180059 reports that lyophilized products normally contain between 5-10% solids but discloses extending this limit to 12% solids. Accommodating higher solid concentrations (10-25%) may be associated with longer drying times, increased complexity of the freezing protocol, and/or the need to increase the surface area of the solution being lyophilized as well as the inclusion of substances such as dextrose, mannitol and dextran. See, e.g., Remington's Pharmaceutical Sciences, Chapter 84, page 1483-1484, 18th Edition, A. R. Gennaro, Editor, Mack Publishing Co., Easton, Pa. 1990.

### SUMMARY

In certain embodiments, the invention relates to pre-lyophilization formulations, lyophilized formulations and methods for preparing, storing and using the same.

In one embodiment, the invention provides a pre-lyophilization solution comprising: a peptide; and a cyclic oligosaccharide (e.g., a cyclodextrin); wherein the solution comprises a solids content of at least 20% w/w, and wherein the cyclic oligosaccharide provides for at least 80% of the solids content.

In another embodiment, the invention provides a pre-lyophilization solution comprising: a peptide; a phospholipid; and a molecule comprising a hydrophilic portion and a liphophilic or hydrophobic portion (e.g., cyclic oligosaccharide, such as a cyclodextrin). In one aspect, the solution comprises a solids content of at least 20% w/w, wherein the phospholipid provides for at least 2% of the solids content (e.g., 0.4% w/w), and wherein the molecule comprising a hydrophilic portion and a liphophilic or hydrophobic portion is present in an amount which solubilizes the phospholipid.

In certain aspects, the peptide is a bioactive peptide. For example, the peptide can be a glucoregulatory peptide or a weight-controlling and/or diet-controlling peptide. In one aspect, the peptide is selected from the group consisting of an incretin, amylin, amylin analog, calcitonin, a calcitonin analog, a leptin, a leptin analog, PYY, a PYY analog, ghrelin and a ghrelin analog, combinations thereof, chimeras, or hybrids thereof. Suitable incretins include, for example, exendin (exendin-3 or exendin-4), an exendin analog, GLP-1, a GLP-1 analog, GIP or a GIP analog or chimeras or hybrids thereof which can include amino acid sequences providing incretin or non-incretin biological activities. Analogs can include agonists or antagonists of a reference peptide, depending on the therapeutic or biological effect desired.

In certain aspects, the peptide is fused or is conjugated to another bioactive peptide and/or can include domains from one or more biologically active peptides. For example, the peptide can be an amylin peptide (or amylin analog) fused or conjugated to a calcitonin peptide (or calcitonin analog).

In one aspect, the solution comprises a preservative which is not benzalkonium chloride and the peptide is an exendin or exendin analog. For example, the solution can comprise one or more parabens.

In another aspect, the solution comprises a polyamino acid.

In a further aspect, the solution does not comprise a cryoprotectant or lyoprotectant.

In still another embodiment, the invention provides a container comprising a pre-lyophilization solution, the solution comprising a peptide wherein the solution comprises a solids content of at least 20% w/w; and wherein the ratio of the fill height to the container internal diameter is greater than 0.50, e.g., 0.75 or greater.

In certain aspects, the container is adapted for use in a delivery system to deliver the peptide to a subject. For example, in one aspect, the container is capable of being sealed with a spray cap, for providing nasal administration of a reconstituted solution after lyophilization.

In a further embodiment, the invention provides a method for preparing a peptide formulation, comprising: providing a pre-lyophilization solution comprising the peptide, wherein the pre-lyophilization solution comprises a solids content of at least 20% w/w; lyophilizing the pre-lyophilization solution comprising the peptide, thereby providing a lyophilized peptide composition; and adding a final volume of aqueous solution to the lyophilized peptide composition, wherein fill volume of the pre-lyophilization solution is 40% of the final volume.

In one aspect, the lyophilization process comprises a freezing stage, a primary drying stage, and a secondary drying stage. In certain aspects, lyophilizing does not include an annealing step.

In one aspect, the primary drying cycle occurs at a temperature which is below the glass transition temperature of an ingredient in the solution which provides the largest contribution to the solids content. For example, the primary drying cycle can occur at a temperature which is below the glass transition temperature of a cyclic oligosaccharide.

In certain aspects, the secondary drying cycle occurs at greater than 25° C.

In other aspects, the peptide is a bioactive peptide and the lyophilized peptide composition is stored at a temperature greater than 4° C. (e.g., greater then 20° C., for example, at 25° C.) and retains biological activity for a period longer than one month, e.g., six months or greater, and in certain aspects, 1 year or longer, 2 years or longer or even 5 years or longer.

In one aspect, lyophilization occurs in less than 48 hours.

In another embodiment, the invention provides a method for preparing a lyophilized peptide composition, comprising: providing a container comprising a pre-lyophilization solution comprising the peptide, wherein the pre-lyophilization solution comprises a solids content of at least 20% w/w; lyophilizing the pre-lyophilization solution comprising the peptide, thereby providing a lyophilized peptide composition; wherein the ratio of the fill height of the pre-lyophilization solution to the container internal diameter is greater than 0.5 (e.g., 0.75 or greater).

In still another embodiment, the invention provides a method for preparing a lyophilized peptide composition, comprising: providing a pre-lyophilization solution comprising the peptide; wherein the pre-lyophilization solution comprises a solids content of at least 20% w/w and further comprises a cyclic oligosaccharide which provides at least 80% of the solids content; and lyophilizing the pre-lyophilization solution, thereby obtaining the lyophilized peptide composition.

In a further embodiment, the invention provides a method for preparing a lyophilized peptide composition, comprising: providing a pre-lyophilization solution comprising the peptide; wherein the pre-lyophilization solution comprises a solids content of at least 20% w/w; and lyophilizing the pre-lyophilization solution, thereby obtaining the lyophilized peptide composition, wherein lyophilization occurs in under 48 hours.

In certain embodiments, the invention also provides methods for storing bioactive peptides. In one aspect, the method comprises preparing a lyophilized peptide composition according to any of the methods described herein and storing the lyophilized peptide composition for at least 48 hours, e.g., at least one month, at least 3 months, or at least six months, at least 1 year, at least 2 years, or at least 5 years. In certain aspects, the lyophilized peptide composition is stored at a temperature above 18° C. (e.g., above 20° C.; for example, at about 25° C.).

Embodiments of the invention include methods for treating a patient treatable with a bioactive peptide comprising administering the peptide in a peptide formulation prepared according to any of the methods described herein.

### BRIEF DESCRIPTION OF THE DRAWINGS

FIG. 1 is a lyophilization cycle trace showing a lyophilization method according to one aspect of the invention.

FIGS. 2A and 2B show the content and purity analysis comparing an exenatide standard to a reconstituted formulation comprising exenatide.

FIGS. 3A and 3B illustrate the content and purity of the formulation over a six month time period at 5° C. and 20° C.

### DETAILED DESCRIPTION

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention.

The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or compositions and/or peptide sequences in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It must be noted that as used herein and in the appended claims, the singular forms “a”, “an”, and “the” include plural referents unless the context clearly dictates otherwise.

It is further noted that invention encompasses both the inclusion and exclusion of any optional elements, and that the indication that an element is optional may be taken as support for the negative limitation that the element can, in certain embodiments, be excluded.

As will be apparent to those of skill in the art upon reading this disclosure, individual embodiments described and illustrated herein may have discrete features which may be readily separated from or combined with the features of any of other embodiment(s) without departing from the scope or spirit of the present invention. Similarly, any recited method can be carried out in the order of events recited or in any other order which is logically possible.

Unless defined otherwise, all technical and scientific terms used herein have the same meaning as commonly understood by one of ordinary skill in the art to which this invention belongs. Still, certain elements are defined below for the sake of clarity and ease of reference.

### Definitions

The term “pharmaceutical formulation” refers to a composition comprising at least one active ingredient (e.g., such as a peptide) in a form and amount which permits the active ingredient to be therapeutically effective. A pharmaceutical formulation can include one or more pharmaceutically acceptable excipients. As used herein the term “ingredient” is used interchangeably with “compound' or “component.”

As used herein, a “pre-lyophilization solution” is a solution comprising at least one active ingredient (e.g., such as a peptide) which can be lyophilized and reconstituted in a form and amount which permits the active ingredient to be therapeutically effective. In certain aspects, the concentration of active ingredient in a pre-lyophilization solution is not a therapeutically effective concentration.

“Pharmaceutically acceptable” excipients or carriers (vehicles, additives) are those which can reasonably be administered to a subject mammal to provide an effective dose of the active ingredient employed. The term pharmaceutical “excipient” and “carrier” are used interchangeably herein.

As used herein, the term “therapeutically effective amount” means an amount of active compound in the composition that will elicit a biological response that is sought in a cell, tissue, system, and/or subject (including a human being), which includes without limitation, alleviation and/or prevention of the symptom(s)of a disorder or condition being treated and/or prevented. As used herein, the term “symptom(s)” refers to any marker(s) of the condition, disease or disorder (collectively referred to herein as a “condition” unless context dictates otherwise) which can be observed directly or indirectly and can include, but is not limited to, physiological response(s) and/or the expression of particular biomarker(s) (e.g., protein(s), peptide(s), nucleic acid(s), metabolites, molecule(s), etc.) associated with a disorder or condition, and/or the progression of a disorder or condition.

As used herein, “treatment” generally refers to an approach for obtaining beneficial or desired results, including clinical results. “Treating” or “palliating” a condition means that the extent and/or undesirable manifestations of the condition, is lessened and/or time course of the progression is slowed or lengthened, as compared to not treating the condition. Beneficial or desired results can include, but are not limited to, alleviation or amelioration of one or more symptoms, diminishment of extent of the condition, stabilizing (i.e., not worsening) the condition, delay or slowing of progression of the condition, amelioration or palliation of the condition, and remission (whether partial or total), whether detectable or undetectable. “Treatment” can also mean prolonging survival as compared to expected survival if not receiving treatment. Further, treating does not necessarily occur by administration of one dose, but can occur upon administration of a series of doses. Thus, a therapeutically effective amount, an amount sufficient to palliate, or an amount sufficient to treat a condition may be administered in one or more doses.

As used herein, “transmucosal administration,” “transmucosal delivery” or equivalent terms refer to administration across any mucosal surface, including, but not limited to oral mucosa, rectal mucosa, urethral mucosa, vaginal mucosa and nasal mucosa, intestinal mucosa and bronchopulmonary mucosa. Oral mucosal administration includes buccal, sublingual and gingival routes of administration. As used herein, transmucosal administration or delivery of a peptide occurs by contacting a mucosal surface with a formulation comprising the peptide and does not include providing a formulation to a mucosal tissue through circulation of the peptide in plasma (e.g., after oral non-mucosal administration and metabolism, for example by ingestion).

The term “solid” generally refers to a non-liquid, non-gaseous structure, and can encompass compositions which are crystalline, amorphous or include a combination of crystalline and amorphous materials.

“Reconstitution time” is the time that is required to rehydrate a lyophilized formulation to provide a clear, particle-free solution.

The “glass transition temperature” (T_{g}) of a composition or a component of the composition is the temperature above which a composition/component changes from a glassy state (e.g., molecules have vibrational motion but have very slow rotational and translational motion) to a liquid. The T_{g} of a composition or component of the composition can be determined using methods known in the art, for example, by differential scanning calorimetry. See, e.g., Angell, C A. Science 1995; 267:1924-1935 and Wolanczy J P. Cryo-Letters 1989; 10:73-76.

As used herein, a “stable lyophilized formulation” is one in which the active ingredient (e.g., such as a bioactive peptide) substantially retains its physical stability, chemical stability and/or biological activity upon storage.

The term amino acid” or “amino acid residue” refers to a natural amino acid, unnatural amino acid, and modified amino acid residue. Unless stated to the contrary, any reference to an amino acid, generally or specifically by name, includes reference to both the D and the L stereoisomers if their structure allow such stereoisomeric forms. Natural amino acids include: alanine (Ala), arginine (Arg), asparagine (Asn), aspartic acid (Asp), cysteine (Cys), glutamine (Gin), glutamic acid (Glu), glycine (Gly), histidine (His), isoleucine (Ile), leucine (Leu), Lysine (Lys), methionine (Met), phenylalanine (Phe), proline (Pro), serine (Ser), threonine (Thr), tryptophan (Trp), tyrosine (Tyr) and valine (Val). Unnatural amino acids include, but are not limited to: homolysine, homoarginine, homoserine, azetidinecarboxylic acid, 2-aminoadipic acid, 3-aminoadipic acid, beta-alanine, aminopropionic acid, 2-aminobutyric acid, 4-aminobutyric acid, 6-aminocaproic acid, 2-aminoheptanoic acid, 2-aminoisobutyric acid, 3-aminoisbutyric acid, 2-aminopimelic acid, tertiary-butyiglycine, 2,4-diaminoisobutyric acid, desmosine, 2,2′-diaminopimeiic acid, 2,3-diaminopropionic acid, N-ethyigiycine, N-ethylasparagine, homoproline, hydroxylysine, allo-hydroxylysine, 3-hydroxyproline, 4-hydroxyproline, isodesmosine, ailo-isoleucine, N-methyialanine, N-methylglycine, N-methylisoieucine, N-methylpentylglycine, N-methylvaline, naphthaianine, norvaline, norleucine, omithine, pentylglycine, pipecolic acid and thioproline. Modified amino acid residues include, but are not limited to those which are chemically blocked, reversibly or irreversibly, or chemically modified on their N-terminal amino group or their side chain groups, as for example, N-methylated D and L amino acids or residues wherein the side chain functional groups are chemically modified to another functional group. For example, modified amino acids include without limitation, methionine sulfoxide; methionine sulfone; aspartic acid-(beta-methyl ester), a modified amino acid of aspartic acid; N-ethylglycine, a modified amino acid of glycine; or alanine carboxamide, and a modified amino acid of alanine. Additional residues that can be incorporated are described in Sandberg et al., J. Med. Chem. 1998; 41: 2481-91. In certain aspects, unnatural amino acids are included at sites of protease cleavage (e.g., such as a cleavage site for DPP-IV) to thereby provide resistance against cleavage.

As used herein, the terms “protein”, “polypeptide” or “peptide” include any molecule that comprises five or more amino acids. It is well known in the art that proteins may undergo modification, including post-translational modifications, such as, but not limited to, disulfide bond formation, glycosylation, phosphorylation, or oligomerization. Thus, as used herein, the term “protein” or “peptide” includes any protein or peptide that is modified by any biological or non-biological process. In certain contexts, as used herein, a “peptide” refers to a polymer comprising less than about 200 amino acid residues, less than about 100 amino acid residues, or less than about 50 amino acid residues. Generally, “peptides” as used herein do not include polyamino acids unless explicitly referred to as such. Also, generally, unless context dictates otherwise, as used herein the term “peptide”, “polypeptide” and “protein” are used herein interchangeably.

The term “polyamino acid” refers to any homopolymer or mixture of homopolymers of a particular amino acid.

As used herein, an “analog” refers to a peptide whose sequence was derived from that of a base reference peptide, e.g., (amylin, calcitonin, PP, PYY, GLP-1, exendin, etc.), and includes insertions, substitutions, extensions, and/or deletions of the reference amino acid sequence, for example having at least 50 or 55% amino acid sequence identity with the base peptide, in other cases, for example, having at least 70%, 80%, 90%, or 95% amino acid sequence identity with the base peptide. Such analogs may comprise conservative or non-conservative amino acid substitutions (including non-natural amino acids and L and D forms). Analogs include compounds having agonist and compounds having antagonist activity. As used herein “analog” refers to bioactive peptides or proteins that are structurally related to a parent peptide by amino acid sequence but which differ from the parent in a characteristic of interest such as bioactivity, solubility, resistance to proteolysis, etc. In certain embodiments, analogs have activities between about 1% to about 10,000%, about 10% to about 1000%, and about 50% to about 500% of the bioactivity of the parental peptide.

Specific types of analogs include amino acid alterations such as deletions, substitutions, additions, and amino acid modifications and derivatizations. A “deletion” refers to the absence of one or more amino acid residue(s) in the related peptide.

An “addition” refers to the presence of one or more amino acid residue(s) in the related peptide. Additions and deletions to a peptide may be at the amino terminus, the carboxy terminus, and/or internal.

Analog peptides can include one or more changes of a “non-essential” amino acid residue compared to a reference peptide. In the context of the invention, a “non-essential” amino acid residue is a residue that can be altered, e.g., deleted or substituted, in the novel amino acid sequence without abolishing or substantially reducing the activity (e.g., the agonist or antagonist activity) of the analog peptide. In certain embodiments, such analogs can include deletions, additions or substitutions of 1-10 or more non-essential amino acid residues without abolishing or substantially reducing the activity of the polypeptide. In one aspect, an analog has greater than 50%, greater than 55% or greater than 60% amino acid identity to a reference peptide. In one aspect, an analog is an agonist of its reference peptide. In antother aspect, an analog is an antagonist of its reference peptide.

A “substitution” refers to the replacement of one or more amino acid residue(s) by another amino acid residue(s) in the peptide. Analogs can contain different combinations of alterations including more than one alteration and different types of alterations. Substitutions include conservative amino acid substitutions. A “conservative amino acid substitution” is one in which the amino acid residue is replaced with an amino acid residue having a similar side chain or similar physicochemical characteristics (e.g., electrostatic, hydrogen bonding, isosteric, hydrophobic features). The amino acids may be naturally occurring or nonnatural (unnatural). Families of amino acid residues having similar side chains are known in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, methionine, cysteine), nonpolar side chains (e.g., alanine, valine, leucine, isoleucine, proline, phenylalanine, tryptophan), branched side chains (e.g., threonine, valine, isoleucine) and aromatic side chains (e.g., tyrosine, phenylalanine, tryptophan, histidine).

A “derivative” refers to a reference peptide or analog, as described above, having a chemical modification of one or more of its amino acid side groups, α-carbon atoms, terminal amino group, or terminal carboxylic acid group.

A “modification” includes, but is not limited to, adding chemical moieties, creating new bonds, and removing chemical moieties. Modifications at amino acid side groups include, without limitation, acylation of lysine 8-amino groups, N-alkylation of arginine, histidine, or lysine, alkylation of glutamic or aspartic carboxylic acid groups, and deamidation of glutamine or asparagine. Modifications of the terminal amino include, without limitation, the desamino, N-lower alkyl, N-di-lower alkyl, and N-acyl modifications. Modifications of the terminal amino include, without limitation, the desamino, N-lower alkyl, N-di-lower alkyl, and N-acyl modifications, such as alkyl acyls, branched alkylacyls, alkylaryl-acyls. Modifications of the terminal carboxy group include, without limitation, the amide, lower alkyl amide, dialkyl amide, arylamide, alkylarylamide and lower alkyl ester modifications. Lower alkyl is C₁-C₄ alkyl. Furthermore, one or more side groups, or terminal groups, may be protected by protective groups known to the ordinarily-skilled synthetic chemist. The α-carbon of an amino acid may be mono-or dimethylated. In certain aspects, modification includes the addition of dicarboxylic acid moieties, fatty acid molecules, weight-enhancing molecules (e.g., such as polyethylene glycol, albumin, gelatin, and the like), carbohydrates (e.g., dextran, saccharides, sialated saccharides, such as monosialated pentasaccharides, and the like), moieties for modifying susceptibility to proteolysis (e.g., by a DPP-IV enzyme), moieties for modifying immunogenicity, antibody molecules or fragments thereof, polymers, and the like. See Ferguson et al., Annu. Rev. Biochem-57:285-320, 1988).

“Percent identity” can be determined as is known in the art. “Identity” measures the percent of identical matches between the smaller of two or more sequences with gap alignments (if any) addressed by a particular mathematical model or computer pro gram (i.e., “algorithms”). In certain aspects, identity can be determined by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis of homology and identity, such described in Schwartz, R. M. and Dayhoff, M. O. In Atlas of Protein Sequences and Structure, (M. O. Dayhoff, ed.), 1979; 5(3):353-358, National Biomedical Research Foundation, Washington, D.C., USA., for example, which adapts the local homology algorithm of Smith and Waterman (Advances in Appl. Math. 1981; 2:482-489) for peptide analysis. The Bestfit program (Wisconsin Sequence Analysis Package, Version 8 for Unix, Genetics Computer Group, University Research Park, 575 Science Drive, Madison, Wis. 53711) also uses local homology algorithm of Smith and Waterman, supra, to find the best segment of similarity between two sequences. These programs are readily utilized with the default parameters recommended by the provider of these programs. Other programs are known in the art and can be used, including, without limitation, BLASTP, e.g., using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. See, e.g., Karlin S and Altschul S F, Proc. Natl. Acad. of Sci. USA 87: 2264-2268, 1990 and Altschul S F, et al. Nucleic Acids Res. 1997; 25:3389-3402. Additional parameters that can be used include the following: Algorithm: Needleman et al., J. Mol. Biol, 1970; 48:443-453 1970; Comparison matrix: BLOSUM 62 from Henikoff et al., Proc. Natl. Acad. Sci. USA, 1992; 89:10915-10919; Gap Penalty: 12, Gap Length Penalty: 4, Threshold of Similarity: 0. Other exemplary algorithms, gap opening penalties, gap extension penalties, comparison matrices, thresholds of similarity, etc. may be used, including those set forth in the Program Manual, Wisconsin Package, Version 9, September, 1997. The particular choices to be made will be apparent to those of skill in the art and will depend on the specific comparison to be made, for example, whether the comparison is between given pairs of sequences (in which case GAP or BestFit are generally preferred) or between one sequence and a large database of sequences (in which case FASTA or BLASTA are preferred).

By “agonist” is meant a compound which elicits a biological activity of a reference peptide. In certain aspects, an agonist has a potency better than the reference peptide, or within five orders of magnitude (plus or minus) of potency compared to the reference peptide, when evaluated by art-known measures, e.g., such as receptor binding/competition studies. In one aspect, an agonist will bind in such assays with an affinity of greater than about 1 μM, and in certain aspects, with an affinity of greater than about 1-5 nM. An agonist can be a fragment of a reference peptide which retains or displays enhanced potency compared to the reference peptide and/or can be an analog of the reference peptide.

As used herein, the term “bioactive” refers to an ability to elicit a biological response that is sought in a cell, tissue, system, and/or subject (including a human being), e.g., a bioactive peptide is one which can be provided in a therapeutically effective amount. In one aspect, a bioactive peptide has biological activity in at least one in vivo hormonal and/or signaling pathway. In another aspect, an agonist can modulate the therapeutic efficacy, scope, duration of action, physicochemical properties, and/or other pharmacokinetic properties of such biological activity. Biological activity may be evaluated through target receptor binding assays, or through metabolic studies that monitor a physiological indication, and/or through the measurement of relevant biomarkers, as is known in the art.

As used herein “subject” or “patient” refers to any animal including domestic animals such as domestic livestock and companion animals. The terms are also meant to include human beings.

As used herein, a “cyclic oligosaccharide” refers to a polymer of saccharides bound cyclically, e.g., via (α-1,4)-linkages (e.g., such as a cyclodextrin).

As discussed above, in certain embodiments, the invention relates to pre-lyophilization formulations, lyophilized formulations and methods for preparing, storing and using the same.

In one aspect, pre-lyophilization formulations according to the invention include at least one peptide. Peptides can include one or more bioactive peptides, including, without limitation any of: amylin, adrenomedullin (“ADM”), calcitonin (“CT”), calcitonin gene related peptide, (“CGRP”), intermedin (e.g., AFP-6); cholecystokinin (a “CCK peptide”, e.g., such as CCK-4, CCK-5, CCK-8, CCK-33), leptin, a pancreatic peptide (“PP”), peptide YY (“PYY”), and more generally, an incretin (e.g., glucagon-like peptide-1 (“GLP-1”), glucagon-like peptide 2 (“GLP-2”), exendin (e.g., exendin-3 or exendin-4)), gastric inhibitory peptide (GIP)), oxyntomodulin (OXM), natriuretic peptides (e.g., ANP, BNP, CNP or urodilatin), a urocortin family peptide (e.g., Urocortin I, II, and III or Ucn-2 and -3), a neuromedin family peptide (e.g., neuromedin U or a splice variant thereof), secretin, gastrin releasing peptide/bombesin, ghrelin, a somatotropin, insulin, and combinations thereof. The sequence composition of the peptides can be as expressed in humans or can be species variants thereof, analogs (agonist or antagonist), derivatives, modified, chimeric and/or hybrid forms thereof. In certain aspects, an antagonist of the peptide is useful as a bioactive agent and peptide analogs which are antagonists of a reference peptide are also encompassed within the scope of the invention. For example, the peptide can include a PYY antagonist or a ghrelin anatagonist. As discussed above, in certain aspects, a peptide can include a functional domain from more than one reference peptide. For example, the peptide can include an amylin (or amylin analog) portion and a calcitonin (or calcitonin analog) portion in a single molecule wherein the amylin and caclitonin portion can be linked covalently via an amide bond or via a non-amide linkage. In one aspect, the peptide which combines a plurality of biological domains from different peptides is an amylin agonist.

In one aspect, the bioactive peptide included in the formulation is an adrenomedullin (ADM). For example, the peptide can be one such as disclosed in Hinson et al. Endocrine Reviews 2000; 21 (2) :13 8-167 and in WO2006042242. As discussed above, formulations can also include analog, derivative, modified, chimeric and/or hybrid forms of ADM peptides.

In one aspect, the bioactive peptide is calcitonin (CT). For example, the peptide can comprise the human peptide hormone calcitonin and species variants thereof, including salmon calcitonin (“sCT”). See, e.g., Becker JCEM 2004; 89(4): 1512-1525; Sexton Current Medicinal Chemistry 1999; 6:1067-1093; and WO2006042242. Formulations can also include analog, derivative, modified, chimeric and/or hybrid forms of calcitonin peptides.

In another aspect, the bioactive peptide is a calcitonin gene related peptide or “CGRP”, for example, the human CGRP or a species variant thereof. See, e.g., Wimalawansa (Crit. Rev. Neurobiol. 1997; 11(2-3):167-239 and WO2006042242. Formulations can also include analog, derivative, modified, chimeric and/or hybrid forms of CGRP peptides.

In still another aspect, the bioactive peptide is an intermedin such as AFP-6 or a species variant thereof. See, e.g., WO2006042242. Formulations can also include analog, derivative, modified, chimeric and/or hybrid forms of intermedin peptides.

In a further aspect, the bioactive peptide is cholecystokinin or “CCK”, for example, the human CCK (e.g., CCK 1-33) or a species variant thereof, which may be sulfated or unsulfated. In one aspect, the CCK peptide is pentagastrin (CCK-5 or CCK(29-33)). In another aspect, the CCK peptide is CCK-4 (CCK (30-33). Formulations can also include analog, derivative, modified, chimeric and/or hybrid forms of CCK peptides. CCK peptides are described in Lieverse et al., Ann. N.Y. Acad. Sci. 1994; 713:268-272, Crawley and Corwin, Peptides 1994; 15: 731-755, Walsh, “Gastrointestinal Hormones,” In Physiology of the Gastrointestinal Tract (3d ed. 1994; Raven Press, New York) and WO2005077072, for example.

A bioactive peptide can also include a leptin. By “leptin” is meant the human leptin or a species variant thereof. Leptin is the polypeptide product of the ob gene as described in the International Patent Publication No. WO 96/05309, Pelleymounter et al. Science 1995; 269:540-543, Halaas et al. Science 1995; 269:543-546, and Campfeld et al. Science 1995; 269:546 549. Formulations can also include analog, derivative, modified, chimeric and/or hybrid forms of leptin peptides. Analogs and fragments of leptin are disclosed in U.S. Pat. No. 5,521,283, U. S. Pat. No. 5, 532,336, PCT/US96/22308 and PCT/US96/01471, WO2004039832, WO2003034996, WO 96/05309; WO 96/40912; WO 97/06816; WO 00/20872; WO 97/18833; WO 97/38014; WO 98/08512 and WO 98/28427, U.S. Pat. No. 5,521,283; U.S. Pat. No. 5,525,705; U.S. Pat. No. 5,532,336; U.S. Pat. No. 5,552,522; U.S. Pat. No. 5,552,523; U.S. Pat. No. 5,552,524; U.S. Pat. No. 5,554,727; U.S. Pat. No. 5,559,208; U.S. Pat. No. 5,563,243; U.S. Pat. No. 5,563,244; U.S. Pat. No. 5,563,245; U.S. Pat. No. 5,567,678;

In still another aspect, the bioactive peptide, is a human oxyntomodulin or species variant thereof. In one aspect, an OXM peptide is a 37 amino acid peptide that contains the 29 amino acid sequence of glucagon followed by an 8 amino acid carboxyterminal extension. Formulations can also include analog, derivative, modified, chimeric and/or hybrid forms of OXM peptides. See, e.g., WO2005035761, WO2004062685, US20060189522, and EP795562.

In a further aspect, the bioactive peptide is a ghrelin peptide, e.g., human ghrelin or a species variant thereof. See, e.g., Kojima et al. Nature 1999; 402(6762):656-60; Arvat, et al. J. Endocrirol Divest 2000; 23(8):493-5; Horvath et al. Pharm Des. 2003; 9(17):1383-95; Wren et al. J Cliff Endocrinl Metab 2001; 86(12):5992; Wren et al. Diabetes 2001; 50(11):2540-7; Kamegai et al. Diabetes 2001; 50(11):2438-43; Shintani et al. Diabetes 2001; 50:227-232 and Asakawa et al. Gut 2003; 52(7):947-52. Formulations can also include analog, derivative, modified, chimeric and/or hybrid forms of ghrelin peptides. In certain aspects, the bioactive peptide is a ghrelin analog which is an antagonist of at least one activity of a ghrelin peptide. See, e.g., WO2004009616.

In certain aspects, the bioactive peptide includes a growth hormone. For example, in one aspect, the bioactive peptide comprises somatotropin, a species variant, analog, derivative, modified form, or a chimeric or hybrid form thereof. See, e.g., WO2005066208, WO1996030405, and U.S. Pat. No. 6,916,914.

The bioactive peptide can also include a natriuretic peptide. Natriuretic peptides act in the body to oppose the activity of the renin-angiotensin system. Atrial natriuretic peptide (ANP), is synthesized in the atria; brain-type natriuretic peptide (BNP), is synthesized in the ventricles; and C-type natriuretic peptide (CNP), is synthesized in the brain. See, e.g., WO2004094459, WO2004094460, WO2005019819. Variants of these peptides are described in, for example, WO2005072055. Urodilatin (CCD 95-126) is a natriuretic peptide which can be isolated from human urine (Forsberg G., et al., J. Prot. Chem. 1991; 10:517-526) and differs from the ANP (99-126) peptide by the inclusion of four additional amino acids at the N-terminus. The amino acid sequence and the structure of urodilatin are described in Drummer C., et al., Am. J. Physiol. 1992; 262:744-754. In certain aspects, the bioactive peptide includes a species variant, analog, derivative, modified form, or a chimeric or hybrid form of a natriuretic peptide.

In still other aspects, the bioactive peptide comprises a urocortin family peptide, such as urocortin I, II or III, a species variant thereof, an analog, derivative, modified form, chimeric or hybrid form thereof. See, e.g., EP845035, US20030032587, and U.S. Pat. No. 6,838,274.

The bioactive peptide can also include a bombesin-like peptide or a neuromedin family peptide, a species variant, an analog, derivative, modified form, chimeric or hybrid form thereof. In one aspect, the bioactive peptide is a neuromedin or a splice variant of a neuromedin. See, e.g., WO2002032937, WO2002032937, and WO2006086769.

In certain aspects, the bioactive peptide comprises a human insulin peptide, a species variant, an analog, derivative, modified, chimeric and/or hybrid form thereof. Insulin peptides are known in the art. See, e.g., as described in US20030144181; US2003010498; US20030040601; US 20030004096A1, U.S. Pat. No. 6,551,992; U.S. Pat. No. 6,534,288; U.S. Pat. No. 6,531,448; U.S. Pat. No. RE37,971; US20020198140; U.S. Pat. No. 6,465,426; U.S. Pat. No. 6,444,641, US20020137144; US20020132760; US20020082199; U.S. Pat. No. 6,335,316; U.S. Pat. No. 6,268,335; US 20010041787; US20010041786; US20010039260; US20010036916; US20010007853A1; U.S. Pat. No. 6,051,551A; U.S. Pat. No. 6,034,054; U.S. Pat. No. 5,970,973; U.S. Pat. No. 5,952,297; U.S. Pat. No. 5,922,675; U.S. Pat. No. 5,888,477; U.S. Pat. No. 5,873,358A; U.S. Pat. No. 5,747,642; U.S. Pat. No. 5,693,609; U.S. Pat. No. 5,650,486; U.S. Pat. No. 5,646,242; U.S. Pat. No. 5,597,893; U.S. Pat. No. 5,547,929; U.S. Pat. No. 5,504,188; U.S. Pat. No. 5,474,978; U.S. Pat. No. 5,461,031; U.S. Pat. No. 4,421,685; U.S. Pat. No. 6,221,837; and U.S. Pat. No. 5,177,058.

In one particular embodiment, the bioactive peptide comprises a human incretin or a species variant, an analog, a derivative, modified, chimeric and/or hybrid form thereof.

In one aspect, the bioactive peptide comprises a gastric inhibitory peptide (GIP), or a species variant, an analog, a derivative, modified, chimeric and/or hybrid form thereof. See, e.g., as described in WO2006086769.

In a further aspect, the bioactive peptide comprises an exendin, exendin analog derivative, or a modified, chimeric and/or hybrid form thereof. Examples of suitable exendins include, but are not limited to, exendin-3, exendin-4, exendin-4 acid, exendin-4 (1-30), exendin-4 (1-30) amide, exendin-4 (1-28), exendin-4 (1-28) amide, ¹⁴Leu, ²⁵Phe, exendin-4 amide, and ¹⁴Leu, ²⁵Phe exendin-4 (1-28) amide as well as other bioactive exendins known in the art, such as those described in WO 99/07404, WO 99/25727, WO 99/25728, and WO 01/04156; US 20030087820; US 2002137666; US 2003087821; and U.S. Pat. No. 6,528,486.

Exendins that can be used in the compositions disclosed herein include those described by Formula I (SEQ ID No. 3) which is as follows:

Xaa₁ Xaa₂ Xaa₃ Gly Thr Xaa₆ Xaa₇ Xaa₈ Xaa₉ Xaa₁₀
Ser Lys Gln Xaa₁₄ Glu Glu Glu Ala Val Arg Leu
Xaa₂₂ Xaa₂₃ Xaa₂₄ Xaa₂₅ Leu Lys Asn Gly Gly
Xaa₃₁ Ser Ser Gly Ala Xaa₃₆ Xaa₃₇ Xaa₃₈ Xaa₃₉-Z;

where:

Xaa₁ is His, Arg or Tyr;

Xaa₂ is Ser, Gly, Ala or Thr;

Xaa₃ is Asp or Glu;

Xaa₆ is Phe, Tyr or naphthylalanine;

Xaa₇ is Thr or Ser;

Xaa₈ is Ser or Thr;

Xaa₉ is Asp or Glu;

Xaa₁₀ is Leu, Ile, Val, pentyiglycine or Met;

Xaa₁₄ is Leu, Ile, pentyiglycine, Val or Met;

Xaa₂₂ is Phe, Tyr or naphthylalanine;

Xaa₂₃ is Ile, Val, Leu, pentyiglycine, tert-butylglycine or Met;

Xaa₂₄ is Glu or Asp;

Xaa₂₅ is Trp, Phe, Tyr, or naphthylalanine;

Xaa₃₁, Xaa₃₆, Xaa₃₇ and Xaa₃₈ are independently Pro, homoproline, 3Hyp, 4Hyp, thioproline, N-alkylglycine, N-alkylpentylglycine or N-alkylalanine;

Xaa₃₉ is Ser, Thr or Tyr; and

Z is —OH or —NH2

Examples of additional exendins that can be used in the compositions disclosed herein include those described by Formula II (SEQ ID No. 4) which is as follows:

Xaa₁ Xaa₂ Xaa₃ Gly Xaa₅ Xaa₆ Xaa₇ Xaa₈
Xaa₉ Xaa₁₀ Xaa₁₁ Xaa₁₂ Xaa₁₃ Xaa₁₄ Xaa₁₅
Xaa₁₆ Xaa₁₇ Ala Xaa₁₉ Xaa₂₀ Xaa₂₁ Xaa₂₂
Xaa₂₃ Xaa₂₄ Xaa₂₅ Xaa₂₆ Xaa₂₇ Xaa₂₈-Z₁;

where

Xaa₁ is His, Arg or Tyr;

Xaa₂ is Ser, Gly, Ala or Thr;

Xaa₃ is Ala, Asp or Glu;

Xaa₅ is Ala or Thr;

Xaa₆ is Ala, Phe, Tyr or naphthylalanine;

Xaa₇ is Thr or Ser;

Xaa₈ is Ala, Ser or Thr;

Xaa₉ is Asp or Glu;

Xaa₁₀ is Ala, Leu, Ile, Val, pentylglycine or Met;

Xaa₁₁ is Ala or Ser;

Xaa₁₂ is Ala or Lys;

Xaa₁₃ is Ala or Gln;

Xaa₁₄ is Ala, Leu, Ile, pentylglycine, Val or Met;

Xaa₁₅ is Ala or Glu;

Xaa₁₆ is Ala or Glu;

Xaa₁₇ is Ala or Glu;

Xaa₁₉ is Ala or Val;

Xaa₂₀ is Ala or Arg;

Xaa₂₁ is Ala or Leu;

Xaa₂₂ is Ala, Phe, Tyr or naphthylalanine;

Xaa₂₃ is Ile, Val, Leu, pentylglycine, tert-butylglycine or Met;

Xaa₂₄ is Ala, Glu or Asp;

Xaa₂₅ is Ala, Trp, Phe, Tyr or naphthylalanine;

Xaa₂₆ is Ala or Leu;

Xaa₂₇ is Ala or Lys;

Xaa₂₈ is Ala or Asn;

Z₁ is —OH,
—NH₂,
Gly-Z₂,
Gly Gly-Z₂,
Gly Gly Xaa₃₁-Z₂
Gly Gly Xaa₃₁ Ser-Z₂,
Gly Gly Xaa₃₁ Ser Ser-Z₂,
Gly Gly Xaa₃₁ Ser Ser Gly-Z₂,
Gly Gly Xaa₃₁ Ser Ser Gly Ala-Z₂,
Gly Gly Xaa₃₁ Ser Ser Gly Ala Xaa₃₆-Z₂,
Gly Gly Xaa₃₁ Ser Ser Gly Ala Xaa₃₆ Xaa₃₇-Z₂, or
Gly Gly Xaa₃₁ Ser Ser Gly Ala Xaa₃₆ Xaa₃₇ Xaa₃₈-Z₂;
Xaa₃₁ Xaa₃₆, Xaa₃₇ and Xaa₃₈ are independently Pro, homoproline, 3Hyp, 4Hyp, thioproline, N-alkylglycine, N-alkylpentylglycine or N-alkylalanine; and
Z₂ is —OH or —NH₂;

provided that no more than three of Xaa₃, Xaa₅, Xaa₆, Xaa₈, Xaa₁₀, Xaa₁₁, Xaa₁₂, Xaa₁₃, Xaa₁₄, Xaa₁₅, Xaa₁₆, Xaa₁₇, Xaa₁₉, Xaa₂₀, Xaa₂₁, Xaa₂₄, Xaa₂₅, Xaa₂₆, Xaa₂₇ and Xaa₂₈ are Ala

Additional examples of exendins that are suitable for use in the compositions disclosed herein are those described by Formula III (SEQ ID No. 5) which is as follows:

Xaa₁ Xaa₂ Xaa₃ Xaa₄ Xaa₅ Xaa₆ Xaa₇ Xaa₈
Xaa₉ Xaa₁₀ Xaa₁₁ Xaa₁₂ Xaa₁₃ Xaa₁₄ Xaa₁₅
Xaa₁₆ Xaa₁₇ Ala Xaa₁₉ Xaa₂₀ Xaa₂₁ Xaa₂₂ Xaa₂₃
Xaa₂₄ Xaa₂₅ Xaa₂₆ Xaa₂₇ Xaa₂₈-Z₁;

Xaa₁ is His, Arg, Tyr, Ala, Norval, Val or Norleu;

Xaa₂ is Ser, Gly, Ala or Thr;

Xaa₃ is Ala, Asp or Glu;

Xaa₄ is Ala, Norval, Val, Norleu or Gly;

Xaa₅ is Ala or Thr;

Xaa₆ is Ala, Phe, Tyr or naphthylalanine;

Xaa₇ is Thr or Ser;

Xaa₈ is Ala, Ser or Thr;

Xaa₉ is Ala, Norval, Val, Norleu, Asp or Glu;

Xaa₁₀ is Ala, Leu, Ile, Val, pentylglycine or Met;

Xaa₁₁ is Ala or Ser;

Xaa₁₂ is Ala or Lys;

Xaa₁₃ is Ala or Gln;

Xaa₁₄ is Ala, Leu, Ile, pentylglycine, Val or Met;

Xaa₁₅ is Ala or Glu;

Xaa₁₆ is Ala or Glu;

Xaa₁₇ is Ala or Glu;

Xaa₁₉ is Ala or Val;

Xaa₂₀ is Ala or Arg;

Xaa₂₁ is Ala or Leu;

Xaa₂₂ is Phe, Tyr or naphthylalanine;

Xaa₂₃ is Ile, Val, Leu, pentylglycine, tert-butylglycine or Met;

Xaa₂₄ is Ala, Glu or Asp;

Xaa₂₅ is Ala, Trp, Phe, Tyr or naphthylalanine;

Xaa₂₆ is Ala or Leu;

Xaa₂₇ is Ala or Lys;

Xaa₂₈ is Ala or Asn;

Z₁ is —OH,
—NH₂
Gly-Z₂,
Gly Gly-Z₂,
Gly Gly Xaa₃₁-Z₂,
Gly Gly Xaa₃₁ Ser-Z₂,
Gly Gly Xaa₃₁ Ser Ser-Z₂,
Gly Gly Xaa₃₁ Ser Ser Gly-Z₂,
Gly Gly Xaa₃₁ Ser Ser Gly Ala-Z₂,
Gly Gly Xaa₃₁ Ser Ser Gly Ala Xaa₃₆-Z₂,
Gly Gly Xaa₃₁ Ser Ser Gly Ala Xaa₃₆ Xaa₃₇-Z₂,
Gly Gly Xaa₃₁ Ser Ser Gly Ala Xaa₃₆ Xaa₃₇ Xaa₃₈-Z₂,
or Gly Gly Xaa₃₁ Ser Ser Gly Ala Xaa₃₆ Xaa₃₇ Xaa₃₈ Xaa₃₉-Z₂;

where:
Xaa₃₁, Xaa₃₆, Xaa₃₇ and Xaa₃₈ are independently Pro, homoproline, 3Hyp, 4Hyp, thioproline, N-alkylglycine, N-alkylpentylglycine or N-alkylalanine;
Xaa₃₉ is Ser, Thr or Tyr; and
Z₂ is —OH or —NH₂;
provided that no more than three of Xaa₃, Xaa₄, Xaa₅, Xaa₆, Xaa₈, Xaa₉, Xaa₁₀, Xaa₁₁, Xaa₁₂, Xaa₁₃, Xaa₁₄, Xaa₁₅, Xaa₁₆, Xaa₁₇, Xaa₁₉, Xaa₂₀, Xaa₂₁, Xaa₂₄, Xaa₂₅, Xaa₂₆, Xaa₂₇ and Xaa₂₈ are Ala;

and provided also that, if Xaa₁ is His, Arg or Tyr, then at least one of Xaa₃, Xaa₄ and Xaa₉ is Ala.

Examples of particular exendins, exendin analogs and exendin derivatives that can be used in the compositions described herein, include, but are not limited to those describe in Table 1. In one embodiment, the bioactive peptide or protein is exendin-4.

TABLE 1
Exendins, Exendin Analogs and Exendin Derivatives

| | |
|---|---|
| SEQ | |
| ID NO | Table 1 Sequence |

| | |
|---|---|
| 1 | His Ser Asp Gly Thr Phe Thr Ser Asp Leu Ser Lys Gln Met Glu Glu Glu Ala Val |
| | Arg Leu Phe Ile Glu Trp Leu Lys Asn Gly Gly Pro Ser Ser Gly Ala Pro Pro Pro |
| | Ser |
| 2 | His Gly Glu Gly Thr Phe Thr Ser Asp Leu Ser Lys Gln Met Glu Glu Glu Ala Val |
| | Arg Leu Phe Ile Glu Trp Leu Lys Asn Gly Gly Pro Ser Ser Gly Ala Pro Pro Pro |
| | Ser |
| 6 | His Gly Glu Gly Thr Phe Thr Ser Asp Leu Ser Lys Gln Met Glu Glu Glu Ala Val |
| | Arg Leu Phe Ile Glu Trp Leu Lys Asn Gly Gly |
| 7 | His Gly Glu Gly Thr Phe Thr Ser Asp Leu Ser Lys Gln Met Glu Glu Glu Ala Val |
| | Arg Leu Phe Ile Glu Trp Leu Lys Asn Gly Gly-NH₂ |
| 8 | His Gly Glu Gly Thr Phe Thr Ser Asp Leu Ser Lys Gln Leu Glu Glu Glu Ala Val |
| | Arg Leu Ala Ile Glu Phe Leu Lys Asn-NH₂ |
| 9 | His Gly Glu Gly Thr Phe Thr Ser Asp Leu Ser Lys Gln Leu Glu Glu Glu Ala Val |
| | Arg Leu Phe Ile Glu Phe Leu Lys Asn Gly Gly Pro Ser Ser Gly Ala Pro Pro Pro |
| | Ser-NH₂ |
| 10 | His Gly Glu Gly Thr Phe Thr Ser Asp Leu Ser Lys Gln Leu Glu Glu Glu Ala Val |
| | Arg Leu Phe Ile Glu Trp Leu Lys Asn Gly Gly Pro Ser Ser Gly Ala Pro Pro Pro |
| | Ser NH₂ |
| 11 | His Gly Glu Gly Thr Phe Thr Ser Asp Leu Ser Lys Gln Met Glu Glu Glu Ala Val |
| | Arg Leu Phe Ile Glu Phe Leu Lys Asn Gly Gly Pro Ser Ser Gly Ala Pro Pro Pro |
| | Ser NH₂ |
| 12 | Tyr Gly Glu Gly Thr Phe Thr Ser Asp Leu Ser Lys Gln Met Glu Glu Glu Ala Val |
| | Arg Leu Phe Ile Glu Trp Leu Lys Asn Gly Gly Pro Ser Ser Gly Ala Pro Pro Pro |
| | Ser NH₂ |
| 13 | His Gly Glu Gly Thr Phe Thr Ser Asp Leu Ser Lys Gln Met Glu Glu Glu Ala Val |
| | Arg Leu Phe Ile Glu Trp Leu Lys Asn Gly Gly Pro Ser Ser Gly Ala Pro Pro Pro |
| | Tyr NH₂ |
| 14 | His Gly Asp Gly Thr Phe Thr Ser Asp Leu Ser Lys Gln Met Glu Glu Glu Ala Val |
| | Arg Leu Phe Ile Glu Trp Leu Lys Asn Gly Gly Pro Ser Ser Gly Ala Pro Pro Pro |
| | Ser NH₂ |
| 15 | His Gly Glu Gly Thr napthylAla Thr Ser Asp Leu Ser Lys Gln Met Glu Glu Glu |
| | Ala Val Arg Leu Phe Ile Glu Trp Leu Lys Asn Gly Gly Pro Ser Ser Gly Ala Pro |
| | Pro Pro Ser NH₂ |
| 16 | His Gly Glu Gly Thr Phe Ser Ser Asp Leu Ser Lys Gln Met Glu Glu Glu Ala Val |
| | Arg Leu Phe Ile Glu Trp Leu Lys Asn Gly Gly Pro Ser Ser Gly Ala Pro Pro Pro |
| | Ser NH₂ |
| 17 | His Gly Glu Gly Thr Phe Ser Thr Asp Leu Ser Lys Gln Met Glu Glu Glu Ala Val |
| | Arg Leu Phe Ile Glu Trp Leu Lys Asn Gly Gly Pro Ser Ser Gly Ala Pro Pro Pro |
| | Ser NH₂ |
| 18 | His Gly Glu Gly Thr Phe Thr Thr Asp Leu Ser Lys Gln Met Glu Glu Glu Ala Val |
| | Arg Leu Phe Ile Glu Trp Leu Lys Asn Gly Gly Pro Ser Ser Gly Ala Pro Pro Pro |
| | Ser NH₂ |
| 19 | His Gly Glu Gly Thr Phe Thr Ser Glu Leu Ser Lys Gln Met Glu Glu Glu Ala Val |
| | Arg Leu Phe Ile Glu Trp Leu Lys Asn Gly Gly Pro Ser Ser Gly Ala Pro Pro Pro |
| | Ser NH₂ |
| 20 | His Gly Glu Gly Thr Phe Thr Ser Asp pentylGly Ser Lys Gln Met Glu Glu Glu |
| | Ala Val Arg Leu Phe Ile Glu Trp Leu Lys Asn Gly Gly Pro Ser Ser Gly Ala Pro |
| | Pro Pro Ser NH |
| 21 | His Gly Glu Gly Thr Phe Thr Ser Asp pentylGly Ser Lys Gln Leu Glu Glu Glu |
| | Ala Val Arg Leu Phe Ile Glu Phe Leu Lys Asn Gly Gly Pro Ser Ser Gly Ala Pro |
| | Pro Pro Ser NH₂ |
| 22 | His Gly Glu Gly Thr Phe Thr Ser Asp Leu Ser Lys Gln pentylGly Glu Glu Glu |
| | Ala Val Arg Leu Phe Ile Glu Trp Leu Lys Asn Gly Gly Pro Ser Ser Gly Ala Pro |
| | Pro Pro Ser NH₂ |
| 23 | His Gly Glu Gly Thr Phe Thr Ser Asp Leu Ser Lys Gln pentylGly Glu Glu Glu |
| | Ala Val Arg Leu Phe Ile Glu Phe Leu Lys Asn Gly Gly Pro Ser Ser Gly Ala Pro |
| | Pro Pro Ser NH₂ |
| 24 | His Gly Glu Gly Thr Phe Thr Ser Asp Leu Ser Lys Gln Met Glu Glu Glu Ala Val |
| | Arg Leu napthylAla Ile Glu Trp Leu Lys Asn Gly Gly Pro Ser Ser Gly Ala Pro |
| | Pro Pro Ser NH₂ |
| 25 | His Gly Glu Gly Thr Phe Thr Ser Asp Leu Ser Lys Gln Met Glu Glu Glu Ala Val |
| | Arg Leu Phe Val Glu Trp Leu Lys Asn Gly Gly Pro Ser Ser Gly Ala Pro Pro Pro |
| | Ser NH₂ |
| 26 | His Gly Glu Gly Thr Phe Thr Ser Asp Leu Ser Lys Gln Leu Glu Glu Glu Ala Val |
| | Arg Leu Phe Val Glu Phe Leu Lys Asn Gly Gly Pro Ser Ser Gly Ala Pro Pro Pro |
| | Ser NH₂ |
| 27 | His Gly Glu Gly Thr Phe Thr Ser Asp Leu Ser Lys Gln Met Glu Glu Glu Ala Val |
| | Arg Leu Phe tbutylGly Glu Trp Leu Lys Asn Gly Gly Pro Ser Ser Gly Ala Pro |
| | Pro Pro Ser NH₂ |
| 28 | His Gly Glu Gly Thr Phe Thr Ser Asp Leu Ser Lys Gln Leu Glu Glu Glu Ala Val |
| | Arg Leu Phe tbutylGly Glu Phe Leu Lys Asn Gly Gly Pro Ser Ser Gly Ala Pro |
| | Pro Pro Ser NH₂ |
| 29 | His Gly Glu Gly Thr Phe Thr Ser Asp Leu Ser Lys Gln Met Glu Glu Glu Ala Val |
| | Arg Leu Phe Ile Asp Trp Leu Lys Asn Gly Gly Pro Ser Ser Gly Ala Pro Pro Pro |
| | Ser NH₂ |
| 30 | His Ala Glu Gly Thr Phe Thr Ser Asp Leu Ser Lys Gln Leu Glu Glu Glu Ala Val |
| | Arg Leu Phe Ile Glu Phe Leu Lys Asn Gly Gly Pro Ser Ser Gly Ala Pro Pro Pro |
| | Ser NH₂ |
| 31 | His Gly Glu Gly Thr Phe Thr Ser Asp Leu Ser Lys Gln Met Glu Glu Glu Ala Val |
| | Arg Leu Phe Ile Glu Trp Leu Lys Asn Gly Gly thioPro Ser Ser Gly Ala thioPro |
| | thioPro thioPro Ser NH₂ |
| 32 | His Gly Glu Gly Thr Phe Thr Ser Asp Leu Ser Lys Gln Met Glu Glu Glu Ala Val |
| | Arg Leu Phe Ile Glu Trp Leu Lys Asn Gly Gly Pro Ser Ser Gly Ala thioPro |
| | thioPro thioPro Ser NH₂ |
| 33 | His Gly Glu Gly Thr Phe Thr Ser Asp Leu Ser Lys Gln Met Glu Glu Glu Ala Val |
| | Arg Leu Phe Ile Glu Trp Leu Lys Asn Gly Gly homoPro Ser Ser Gly Ala |
| | homoPro homoPro homoPro Ser NH₂ |
| 34 | His Gly Glu Gly Thr Phe Thr Ser Asp Leu Ser Lys Gln Met Glu Glu Glu Ala Val |
| | Arg Leu Phe Ile Glu Trp Leu Lys Asn Gly Gly Pro Ser Ser Gly Ala homoPro |
| | homoPro homoPro Ser NH₂ |
| 35 | His Gly Glu Gly Thr Phe Thr Ser Asp Leu Ser Lys Gln Leu Glu Glu Glu Ala Val |
| | Arg Leu Phe Ile Glu Phe Leu Lys Asn Gly Gly thioPro Ser Ser Gly Ala thioPro |
| | thioPro thioPro Ser NH₂ |
| 36 | His Gly Glu Gly Thr Phe Thr Ser Asp Leu Ser Lys Gln Leu Glu Glu Glu Ala Val |
| | Arg Leu Phe Ile Glu Phe Leu Lys Asn Gly Gly homoPro Ser Ser Gly Ala |
| | homoPro homoPro homoPro Ser NH₂ |
| 37 | His Gly Glu Gly Thr Phe Thr Ser Asp Leu Ser Lys Gln Met Glu Glu Glu Ala Val |
| | Arg Leu Phe Ile Glu Trp Leu Lys Asn Gly Gly NmethylAla Ser Ser Gly Ala |
| | NmethylAla NmethylAla NmethylAla Ser NH₂ |
| 38 | His Gly Glu Gly Thr Phe Thr Ser Asp Leu Ser Lys Gln Met Glu Glu Glu Ala Val |
| | Arg Leu Phe Ile Glu Trp Leu Lys Asn Gly Gly Pro Ser Ser Gly Ala NmethylAla |
| | NmethylAla NmethylAla Ser NH₂ |
| 39 | His Gly Glu Gly Thr Phe Thr Ser Asp Leu Ser Lys Gln Leu Glu Glu Glu Ala Val |
| | Arg Leu Phe Ile Glu Phe Leu Lys Asn Gly Gly NmethylAla Ser Ser Gly Ala |
| | NmethylAla NmethylAla NmethylAla Ser NH₂ |
| 40 | His Gly Glu Gly Thr Phe Thr Ser Asp Leu Ser Lys Gln Met Glu Glu Glu Ala Val |
| | Arg Leu Phe Ile Glu Trp Leu Lys Asn-NH₂ |
| 41 | His Gly Glu Gly Thr Phe Thr Ser Asp Leu Ser Lys Gln Leu Glu Glu Glu Ala Val |
| | Arg Leu Phe Ile Glu Phe Leu Lys Asn-NH₂ |
| 42 | His Ala Glu Gly Thr Phe Thr Ser Asp Leu Ser Lys Gln Leu Glu Glu Glu Ala Val |
| | Arg Leu Phe Ile Glu Phe Leu Lys Asn-NH₂ |
| 43 | His Gly Glu Gly Ala Phe Thr Ser Asp Leu Ser Lys Gln Leu Glu Glu Glu Ala Val |
| | Arg Leu Phe Ile Glu Phe Leu Lys Asn-NH₂ |
| 44 | His Gly Glu Gly Thr Ala Thr Ser Asp Leu Ser Lys Gln Leu Glu Glu Glu Ala Val |
| | Arg Leu Phe Ile Glu Phe Leu Lys Asn-NH₂ |
| 45 | His Gly Glu Gly Thr Phe Thr Ala Asp Leu Ser Lys Gln Leu Glu Glu Glu Ala Val |
| | Arg Leu Phe Ile Glu Phe Leu Lys Asn-NH₂ |
| 46 | His Gly Glu Gly Thr Phe Thr Ser Asp Ala Ser Lys Gln Leu Glu Glu Glu Ala Val |
| | Arg Leu Phe Ile Glu Phe Leu Lys Asn-NH₂ |
| 47 | His Gly Glu Gly Thr Phe Thr Ser Asp Leu Ala Lys Gln Leu Glu Glu Glu Ala Val |
| | Arg Leu Phe Ile Glu Phe Leu Lys Asn-NH₂ |
| 48 | His Gly Glu Gly Thr Phe Thr Ser Asp Leu Ser Ala Gln Leu Glu Glu Glu Ala Val |
| | Arg Leu Phe Ile Glu Phe Leu Lys Asn-NH₂ |
| 49 | His Gly Glu Gly Thr Phe Thr Ser Asp Leu Ser Lys Ala Leu Glu Glu Glu Ala Val |
| | Arg Leu Phe Ile Glu Phe Leu Lys Asn-NH₂ |
| 50 | His Gly Glu Gly Thr Phe Thr Ser Asp Leu Ser Lys Gln Ala Glu Glu Glu Ala Val |
| | Arg Leu Phe Ile Glu Phe Leu Lys Asn-NH₂ |
| 51 | His Gly Glu Gly Thr Phe Thr Ser Asp Leu Ser Lys Gln Leu Ala Glu Glu Ala Val |
| | Arg Leu Phe Ile Glu Phe Leu Lys Asn-NH₂ |
| 52 | His Gly Glu Gly Thr Phe Thr Ser Asp Leu Ser Lys Gln Leu Glu Ala Glu Ala Val |
| | Arg Leu Phe Ile Glu Phe Leu Lys Asn-NH₂ |
| 53 | His Gly Glu Gly Thr Phe Thr Ser Asp Leu Ser Lys Gln Leu Glu Glu Ala Ala Val |
| | Arg Leu Phe Ile Glu Phe Leu Lys Asn-NH₂ |
| 54 | His Gly Glu Gly Thr Phe Thr Ser Asp Leu Ser Lys Gln Leu Glu Glu Glu Ala Ala |
| | Arg Leu Phe Ile Glu Phe Leu Lys Asn-NH₂ |
| 55 | His Gly Glu Gly Thr Phe Thr Ser Asp Leu Ser Lys Gln Leu Glu Glu Glu Ala Val |
| | Ala Leu Phe Ile Glu Phe Leu Lys Asn-NH₂ |
| 56 | His Gly Glu Gly Thr Phe Thr Ser Asp Leu Ser Lys Gln Leu Glu Glu Glu Ala Val |
| | Arg Ala Phe Ile Glu Phe Leu Lys Asn-NH₂ |
| 57 | His Gly Glu Gly Thr Phe Thr Ser Asp Leu Ser Lys Gln Leu Glu Glu Glu Ala Val |
| | Arg Leu Phe Ile Ala Phe Leu Lys Asn-NH₂ |
| 58 | His Gly Glu Gly Thr Phe Thr Ser Asp Leu Ser Lys Gln Leu Glu Glu Glu Ala Val |
| | Arg Leu Phe Ile Glu Ala Leu Lys Asn-NH₂ |
| 59 | His Gly Glu Gly Thr Phe Thr Ser Asp Leu Ser Lys Gln Leu Glu Glu Glu Ala Val |
| | Arg Leu Phe Ile Glu Phe Ala Lys Asn-NH₂ |
| 60 | His Gly Glu Gly Thr Phe Thr Ser Asp Leu Ser Lys Gln Leu Glu Glu Glu Ala Val |
| | Arg Leu Phe Ile Glu Phe Leu Ala Asn-NH₂ |
| 61 | His Gly Glu Gly Thr Phe Thr Ser Asp Leu Ser Lys Gln Leu Glu Glu Glu Ala Val |
| | Arg Leu Phe Ile Glu Phe Leu Lys Ala-NH₂ |
| 62 | His Gly Glu Gly Thr Phe Thr Ser Asp Leu Ser Lys Gln Met Glu Glu Glu Ala Val |
| | Arg Leu Phe Ile Glu Trp Leu Lys Asn Gly Gly Pro Ser Ser Gly Ala Pro Pro Pro- |
| | NH₂ |
| 63 | His Gly Glu Gly Thr Phe Thr Ser Asp Leu Ser Lys Gln Leu Glu Glu Glu Ala Val |
| | Arg Leu Phe Ile Glu Phe Leu Lys Asn Gly Gly Pro Ser Ser Gly Ala Pro Pro Pro- |
| | NH₂ |
| 64 | His Gly Glu Gly Thr Phe Thr Ser Asp Leu Ser Lys Gln Met Glu Glu Glu Ala Val |
| | Arg Leu Phe Ile Glu Trp Leu Lys Asn Gly Gly Pro Ser Ser Gly Ala Pro Pro-NH₂ |
| 65 | His Gly Glu Gly Thr Phe Thr Ser Asp Leu Ser Lys Gln Leu Glu Glu Glu Ala Val |
| | Arg Leu Phe Ile Glu Phe Leu Lys Asn Gly Gly Pro Ser Ser Gly Ala Pro Pro-NH₂ |
| 66 | His Gly Glu Gly Thr Phe Thr Ser Asp Leu Ser Lys Gln Met Glu Glu Glu Ala Val |
| | Arg Leu Phe Ile Glu Trp Leu Lys Asn Gly Gly Pro Ser Ser Gly Ala Pro-NH₂ |
| 67 | His Gly Glu Gly Thr Phe Thr Ser Asp Leu Ser Lys Gln Leu Glu Glu Glu Ala Val |
| | Arg Leu Phe Ile Glu Phe Leu Lys Asn Gly Gly Pro Ser Ser Gly Ala Pro-NH₂ |
| 68 | His Gly Glu Gly Thr Phe Thr Ser Asp Leu Ser Lys Gln Met Glu Glu Glu Ala Val |
| | Arg Leu Phe Ile Glu Trp Leu Lys Asn Gly Gly Pro Ser Ser Gly Ala-NH₂ |
| 69 | His Gly Glu Gly Thr Phe Thr Ser Asp Leu Ser Lys Gln Leu Glu Glu Glu Ala Val |
| | Arg Leu Phe Ile Glu Phe Leu Lys Asn Gly Gly Pro Ser Ser Gly Ala-NH₂ |
| 70 | His Gly Glu Gly Thr Phe Thr Ser Asp Leu Ser Lys Gln Met Glu Glu Glu Ala Val |
| | Arg Leu Phe Ile Glu Trp Leu Lys Asn Gly Gly Pro Ser Ser Gly-NH₂ |
| 71 | His Gly Glu Gly Thr Phe Thr Ser Asp Leu Ser Lys Gln Leu Glu Glu Glu Ala Val |
| | Arg Leu Phe Ile Glu Phe Leu Lys Asn Gly Gly Pro Ser Ser Gly-NH₂ |
| 72 | His Gly Glu Gly Thr Phe Thr Ser Asp Leu Ser Lys Gln Met Glu Glu Glu Ala Val |
| | Arg Leu Phe Ile Glu Trp Leu Lys Asn Gly Gly Pro Ser Ser-NH₂ |
| 73 | His Gly Glu Gly Thr Phe Thr Ser Asp Leu Ser Lys Gln Leu Glu Glu Glu Ala Val |
| | Arg Leu Phe Ile Glu Phe Leu Lys Asn Gly Gly Pro Ser Ser-NH₂ |
| 74 | His Gly Glu Gly Thr Phe Thr Ser Asp Leu Ser Lys Gln Met Glu Glu Glu Ala Val |
| | Arg Leu Phe Ile Glu Trp Leu Lys Asn Gly Gly Pro Ser-NH₂ |
| 75 | His Gly Glu Gly Thr Phe Thr Ser Asp Leu Ser Lys Gln Leu Glu Glu Glu Ala Val |
| | Arg Leu Phe Ile Glu Phe Leu Lys Asn Gly Gly Pro Ser-NH₂ |
| 76 | His Gly Glu Gly Thr Phe Thr Ser Asp Leu Ser Lys Gln Met Glu Glu Glu Ala Val |
| | Arg Leu Phe Ile Glu Trp Leu Lys Asn Gly Gly Pro-NH₂ |
| 77 | His Gly Glu Gly Thr Phe Thr Ser Asp Leu Ser Lys Gln Leu Glu Glu Glu Ala Val |
| | Arg Leu Phe Ile Glu Phe Leu Lys Asn Gly Gly Pro-NH₂ |
| 78 | His Gly Glu Gly Thr Phe Thr Ser Asp Leu Ser Lys Gln Leu Glu Glu Glu Ala Val |
| | Arg Leu Phe Ile Glu Phe Leu Lys Asn Gly Gly-NH₂ |
| 79 | His Gly Glu Gly Thr Phe Thr Ser Asp Leu Ser Lys Gln Met Glu Glu Glu Ala Val |
| | Arg Leu Phe Ile Glu Trp Leu Lys Asn Gly-NH₂ |
| 80 | His Gly Glu Gly Thr Phe Thr Ser Asp Leu Ser Lys Gln Leu Glu Glu Glu Ala Val |
| | Arg Leu Phe Ile Glu Phe Leu Lys Asn Gly-NH₂ |
| 81 | His Gly Glu Gly Thr Phe Thr Ser Asp Leu Ser Lys Gln Met Glu Glu Glu Ala Val |
| | Arg Leu Phe Ile Glu Trp Leu Lys Asn Gly Gly thioPro Ser Ser Gly Ala thioPro |
| | thioPro thioPro-NH₂ |
| 82 | His Gly Glu Gly Thr Phe Thr Ser Asp Leu Ser Lys Gln Met Glu Glu Glu Ala Val |
| | Arg Leu Phe Ile Glu Trp Leu Lys Asn Gly Gly Pro Ser Ser Gly Ala thioPro |
| | thioPro thioPro-NH₂ |
| 83 | His Gly Glu Gly Thr Phe Thr Ser Asp Leu Ser Lys Gln Met Glu Glu Glu Ala Val |
| | Arg Leu Phe Ile Glu Trp Leu Lys Asn Gly Gly NMeala Ser Ser Gly Ala Pro Pro- |
| | NH₂ |
| 84 | His Gly Glu Gly Thr Phe Thr Ser Asp Leu Ser Lys Gln Met Glu Glu Glu Ala Val |
| | Arg Leu Phe Ile Glu Trp Leu Lys Asn Gly Gly NMeala Ser Ser Gly Ala NMeAla |
| | NmeAla-NH₂ |
| 85 | His Gly Glu Gly Thr Phe Thr Ser Asp Leu Ser Lys Gln Met Glu Glu Glu Ala Val |
| | Arg Leu Phe Ile Glu Trp Leu Lys Asn Gly Gly homoPro Ser Ser Gly Ala |
| | homoPro homoPro-NH₂ |
| 86 | His Gly Glu Gly Thr Phe Thr Ser Asp Leu Ser Lys Gln Met Glu Glu Glu Ala Val |
| | Arg Leu Phe Ile Glu Trp Leu Lys Asn Gly Gly homoPro Ser Ser Gly Ala |
| | homoPro-NH₂ |
| 87 | Arg Gly Glu Gly Thr Phe Thr Ser Asp Leu Ser Lys Gln Met Glu Glu Glu Ala Val |
| | Arg Leu Phe Ile Glu Trp Leu Lys Asn Gly Gly Pro Ser Ser Gly Ala-NH₂ |
| 88 | His Gly Asp Gly Thr Phe Thr Ser Asp Leu Ser Lys Gln Met Glu Glu Glu Ala Val |
| | Arg Leu Phe Ile Glu Trp Leu Lys Asn Gly Gly-NH₂ |
| 89 | His Gly Glu Gly Thr NaphthylAla Thr Ser Asp Leu Ser Lys Gln Leu Glu Glu Glu |
| | Ala Val Arg Leu Phe Ile Glu Phe Leu Lys Asn-NH₂ |
| 90 | His Gly Glu Gly Thr Phe Ser Ser Asp Leu Ser Lys Gln Met Glu Glu Glu Ala Val |
| | Arg Leu Phe Ile Glu Trp Leu Lys Asn-NH₂ |
| 91 | His Gly Glu Gly Thr Phe Ser Thr Asp Leu Ser Lys Gln Met Glu Glu Glu Ala Val |
| | Arg Leu Phe Ile Glu Trp Leu Lys Asn-NH₂ |
| 92 | His Gly Glu Gly Thr Phe Thr Ser Glu Leu Ser Lys Gln Met Ala Glu Glu Ala Val |
| | Arg Leu Phe Ile Glu Trp Leu Lys Asn-NH₂ |
| 93 | His Gly Glu Gly Thr Phe Thr Ser Asp pentylGly Ser Lys Gln Leu Glu Glu Glu |
| | Ala Val Arg Leu Phe Ile Glu Phe Leu Lys Asn-NH₂ |
| 94 | His Gly Glu Gly Thr Phe Thr Ser Asp Leu Ser Lys Gln Leu Glu Glu Glu Ala Val |
| | Arg Leu NaphthylAla Ile Glu Phe Leu Lys Asn-NH₂ |
| 95 | His Gly Glu Gly Thr Phe Thr Ser Asp Leu Ser Lys Gln Met Glu Glu Glu Ala Val |
| | Arg Leu Phe tButylGly Glu Trp Leu Lys Asn-NH₂ |
| 96 | His Gly Glu Gly Thr Phe Thr Ser Asp Leu Ser Lys Gln Leu Glu Glu Glu Ala Val |
| | Arg Leu Phe Ile Asp Phe Leu Lys Asn-NH₂ |
| 97 | His Gly Glu Gly Thr Phe Thr Ser Asp Ala Ser Lys Gln Leu Glu Glu Glu Ala Val |
| | Arg Leu Phe Ile Glu Phe Leu Lys Asn Gly Gly Pro Ser Ser-NH₂ |
| 98 | His Gly Glu Gly Thr Phe Thr Ser Asp Ala Ser Lys Gln Met Glu Glu Glu Ala Val |
| | Arg Leu Phe Ile Glu Trp Leu Lys Asn Gly-NH₂ |
| 99 | His Gly Glu Gly Thr Phe Thr Ser Asp Ala Ser Lys Gln Met Glu Glu Glu Ala Val |
| | Arg Leu Phe Ile Glu Trp Leu Lys Asn Gly Gly homoPro Ser Ser Gly Ala |
| | homoPro homoPro-NH₂ |
| 100 | Ala Gly Glu Gly Thr Phe Thr Ser Asp Leu Ser Lys Gln Leu Glu Glu Glu Ala Val |
| | Arg Leu Phe Ile Glu Phe Leu Lys Asn-NH₂ |
| 101 | His Gly Ala Gly Thr Phe Thr Ser Asp Leu Ser Lys Gln Leu Glu Glu Glu Ala Val |
| | Arg Leu Phe Ile Glu Phe Leu Lys Asn-NH₂ |
| 102 | His Gly Glu Ala Thr Phe Thr Ser Asp Leu Ser Lys Gln Leu Glu Glu Glu Ala Val |
| | Arg Leu Phe Ile Glu Phe Leu Lys Asn-NH₂ |
| 103 | His Gly Glu Gly Thr Phe Thr Ser Ala Leu Ser Lys Gln Leu Glu Glu Glu Ala Val |
| | Arg Leu Phe Ile Glu Phe Leu Lys Asn-NH₂ |
| 104 | Ala Gly Glu Gly Thr Phe Thr Ser Asp Leu Ser Lys Gln Met Glu Glu Glu Ala Val |
| | Arg Leu Phe Ile Glu Trp Leu Lys Asn-NH₂ |
| 105 | His Gly Ala Gly Thr Phe Thr Ser Asp Leu Ser Lys Gln Met Glu Glu Glu Ala Val |
| | Arg Leu Phe Ile Glu Trp Leu Lys Asn-NH₂ |
| 106 | His Gly Glu Ala Thr Phe Thr Ser Asp Leu Ser Lys Gln Met Glu Glu Glu Ala Val |
| | Arg Leu Phe Ile Glu Trp Leu Lys Asn-NH₂ |
| 107 | His Gly Glu Gly Thr Phe Thr Ser Ala Leu Ser Lys Gln Met Glu Glu Glu Ala Val |
| | Arg Leu Phe Ile Glu Trp Leu Lys Asn-NH₂ |
| 108 | His Gly Glu Gly Thr Phe Thr Ser Asp Ala Ser Lys Gln Met Glu Glu Glu Ala Val |
| | Arg Leu Phe Ile Glu Trp Leu Lys Asn-NH₂ |
| 109 | Ala Ala Glu Gly Thr Phe Thr Ser Asp Leu Ser Lys Gln Met Glu Glu Glu Ala Val |
| | Arg Leu Phe Ile Glu Trp Leu Lys Asn-NH₂ |
| 110 | Ala Ala Glu Gly Thr Phe Thr Ser Asp Leu Ser Lys Gln Leu Glu Glu Glu Ala Val |
| | Arg Leu Phe Ile Glu Phe Leu Lys Asn-NH₂ |
| 111 | Ala Gly Asp Gly Thr Phe Thr Ser Asp Leu Ser Lys Gln Met Glu Glu Glu Ala Val |
| | Arg Leu Phe Ile Glu Trp Leu Lys Asn-NH₂ |
| 112 | Ala Gly Asp Gly Thr Phe Thr Ser Asp Leu Ser Lys Gln Leu Glu Glu Glu Ala Val |
| | Arg Leu Phe Ile Glu Phe Leu Lys Asn-NH₂ |
| 113 | Ala Gly Asp Gly Ala Phe Thr Ser Asp Leu Ser Lys Gln Met Glu Glu Glu Ala Val |
| | Arg Leu Phe Ile Glu Trp Leu Lys Asn-NH₂ |
| 114 | Ala Gly Asp Gly Ala Phe Thr Ser Asp Leu Ser Lys Gln Leu Glu Glu Glu Ala Val |
| | Arg Leu Phe Ile Glu Phe Leu Lys Asn-NH₂ |
| 115 | Ala Gly Asp Gly Thr NaphthylAla Thr Ser Asp Leu Ser Lys Gln Met Glu Glu Glu |
| | Ala Val Arg Leu Phe Ile Glu Trp Leu Lys Asn-NH₂ |
| 116 | Ala Gly Asp Gly Thr NaphthylAla Thr Ser Asp Leu Ser Lys Gln Leu Glu Glu Glu |
| | Ala Val Arg Leu Phe Ile Glu Phe Leu Lys Asn-NH₂ |
| 117 | Ala Gly Asp Gly Thr Phe Ser Ser Asp Leu Ser Lys Gln Met Glu Glu Glu Ala Val |
| | Arg Leu Phe Ile Glu Trp Leu Lys Asn-NH₂ |
| 118 | Ala Gly Asp Gly Thr Phe Ser Ser Asp Leu Ser Lys Gln Leu Glu Glu Glu Ala Val |
| | Arg Leu Phe Ile Glu Phe Leu Lys Asn-NH₂ |
| 119 | Ala Gly Asp Gly Thr Phe Thr Ala Asp Leu Ser Lys Gln Met Glu Glu Glu Ala Val |
| | Arg Leu Phe Ile Glu Trp Leu Lys Asn-NH₂ |
| 120 | Ala Gly Asp Gly Thr Phe Thr Ala Asp Leu Ser Lys Gln Leu Glu Glu Glu Ala Val |
| | Arg Leu Phe Ile Glu Phe Leu Lys Asn-NH₂ |
| 121 | Ala Gly Asp Gly Thr Phe Thr Ser Ala Leu Ser Lys Gln Met Glu Glu Glu Ala Val |
| | Arg Leu Phe Ile Glu Trp Leu Lys Asn-NH₂ |
| 122 | Ala Gly Asp Gly Thr Phe Thr Ser Ala Leu Ser Lys Gln Leu Glu Glu Glu Ala Val |
| | Arg Leu Phe Ile Glu Phe Leu Lys Asn-NH₂ |
| 123 | Ala Gly Asp Gly Thr Phe Thr Ser Glu Leu Ser Lys Gln Met Glu Glu Glu Ala Val |
| | Arg Leu Phe Ile Glu Trp Leu Lys Asn-NH₂ |
| 124 | Ala Gly Asp Gly Thr Phe Thr Ser Glu Leu Ser Lys Gln Leu Glu Glu Glu Ala Val |
| | Arg Leu Phe Ile Glu Phe Leu Lys Asn-NH₂ |
| 125 | Ala Gly Asp Gly Thr Phe Thr Ser Asp Ala Ser Lys Gln Met Glu Glu Glu Ala Val |
| | Arg Leu Phe Ile Glu Trp Leu Lys Asn-NH₂ |
| 126 | Ala Gly Asp Gly Thr Phe Thr Ser Asp Ala Ser Lys Gln Leu Glu Glu Glu Ala Val |
| | Arg Leu Phe Ile Glu Phe Leu Lys Asn-NH₂ |
| 127 | Ala Gly Asp Gly Thr Phe Thr Ser Asp pentylGly Ser Lys Gln Met Glu Glu Glu |
| | Ala Val Arg Leu Phe Ile Glu Trp Leu Lys Asn-NH₂ |
| 128 | Ala Gly Asp Gly Thr Phe Thr Ser Asp pentylGly Ser Lys Gln Leu Glu Glu Glu |
| | Ala Val Arg Leu Phe Ile Glu Phe Leu Lys Asn-NH₂ |
| 129 | Ala Gly Asp Gly Thr Phe Thr Ser Asp Leu Ala Lys Gln Met Glu Glu Glu Ala Val |
| | Arg Leu Phe Ile Glu Trp Leu Lys Asn-NH₂ |
| 130 | Ala Gly Asp Gly Thr Phe Thr Ser Asp Leu Ala Lys Gln Leu Glu Glu Glu Ala Val |
| | Arg Leu Phe Ile Glu Phe Leu Lys Asn-NH₂ |
| 131 | Ala Gly Asp Gly Thr Phe Thr Ser Asp Leu Ser Ala Gln Met Glu Glu Glu Ala Val |
| | Arg Leu Phe Ile Glu Trp Leu Lys Asn-NH₂ |
| 132 | Ala Gly Asp Gly Thr Phe Thr Ser Asp Leu Ser Ala Gln Leu Glu Glu Glu Ala Val |
| | Arg Leu Phe Ile Glu Phe Leu Lys Asn-NH₂ |
| 133 | Ala Gly Asp Gly Thr Phe Thr Ser Asp Leu Ser Lys Ala Met Glu Glu Glu Ala Val |
| | Arg Leu Phe Ile Glu Trp Leu Lys Asn-NH₂ |
| 134 | Ala Gly Asp Gly Thr Phe Thr Ser Asp Leu Ser Lys Ala Leu Glu Glu Glu Ala Val |
| | Arg Leu Phe Ile Glu Phe Leu Lys Asn-NH₂ |
| 135 | Ala Gly Asp Gly Thr Phe Thr Ser Asp Leu Ser Lys Gln Ala Glu Glu Glu Ala Val |
| | Arg Leu Phe Ile Glu Trp Leu Lys Asn-NH₂ |
| 136 | Ala Gly Asp Gly Thr Phe Thr Ser Asp Leu Ser Lys Gln Ala Glu Glu Glu Ala Val |
| | Arg Leu Phe Ile Glu Phe Leu Lys Asn-NH₂ |
| 137 | Ala Gly Asp Gly Thr Phe Thr Ser Asp Leu Ser Lys Gln pentylGly Glu Glu Glu |
| | Ala Val Arg Leu Phe Ile Glu Trp Leu Lys Asn-NH₂ |
| 138 | Ala Gly Asp Gly Thr Phe Thr Ser Asp Leu Ser Lys Gln pentylGly Glu Glu Glu |
| | Ala Val Arg Leu Phe Ile Glu Phe Leu Lys Asn-NH₂ |
| 139 | Ala Gly Asp Gly Thr Phe Thr Ser Asp Leu Ser Lys Gln Met Ala Glu Glu Ala Val |
| | Arg Leu Phe Ile Glu Trp Leu Lys Asn-NH₂ |
| 140 | Ala Gly Asp Gly Thr Phe Thr Ser Asp Leu Ser Lys Gln Leu Ala Glu Glu Ala Val |
| | Arg Leu Phe Ile Glu Phe Leu Lys Asn-NH₂ |
| 141 | Ala Gly Asp Gly Thr Phe Thr Ser Asp Leu Ser Lys Gln Met Glu Ala Glu Ala Val |
| | Arg Leu Phe Ile Glu Trp Leu Lys Asn-NH₂ |
| 142 | Ala Gly Asp Gly Thr Phe Thr Ser Asp Leu Ser Lys Gln Leu Glu Ala Glu Ala Val |
| | Arg Leu Phe Ile Glu Phe Leu Lys Asn-NH₂ |
| 143 | Ala Gly Asp Gly Thr Phe Thr Ser Asp Leu Ser Lys Gln Met Glu Glu Ala Ala Val |
| | Arg Leu Phe Ile Glu Trp Leu Lys Asn-NH₂ |
| 144 | Ala Gly Asp Gly Thr Phe Thr Ser Asp Leu Ser Lys Gln Leu Glu Glu Ala Ala Val |
| | Arg Leu Phe Ile Glu Phe Leu Lys Asn-NH₂ |
| 145 | Ala Gly Asp Gly Thr Phe Thr Ser Asp Leu Ser Lys Gln Met Glu Glu Glu Ala Ala |
| | Arg Leu Phe Ile Glu Trp Leu Lys Asn-NH₂ |
| 146 | Ala Gly Asp Gly Thr Phe Thr Ser Asp Leu Ser Lys Gln Leu Glu Glu Glu Ala Ala |
| | Arg Leu Phe Ile Glu Phe Leu Lys Asn-NH₂ |
| 147 | Ala Gly Asp Gly Thr Phe Thr Ser Asp Leu Ser Lys Gln Met Glu Glu Glu Ala Val |
| | Ala Leu Phe Ile Glu Trp Leu Lys Asn-NH₂ |
| 148 | Ala Gly Asp Gly Thr Phe Thr Ser Asp Leu Ser Lys Gln Leu Glu Glu Glu Ala Val |
| | Ala Leu Phe Ile Glu Phe Leu Lys Asn-NH₂ |
| 149 | Ala Gly Asp Gly Thr Phe Thr Ser Asp Leu Ser Lys Gln Met Glu Glu Glu Ala Val |
| | Arg Ala Phe Ile Glu Trp Leu Lys Asn-NH₂ |
| 150 | Ala Gly Asp Gly Thr Phe Thr Ser Asp Leu Ser Lys Gln Leu Glu Glu Glu Ala Val |
| | Arg Ala Phe Ile Glu Phe Leu Lys Asn-NH₂ |
| 151 | Ala Gly Asp Gly Thr Phe Thr Ser Asp Leu Ser Lys Gln Met Glu Glu Glu Ala Val |
| | Arg Leu Naphthylala Ile Glu Trp Leu Lys Asn-NH₂ |
| 152 | Ala Gly Asp Gly Thr Phe Thr Ser Asp Leu Ser Lys Gln Leu Glu Glu Glu Ala Val |
| | Arg Leu Naphthylala Ile Glu Phe Leu Lys Asn-NH₂ |
| 153 | Ala Gly Asp Gly Thr Phe Thr Ser Asp Leu Ser Lys Gln Met Glu Glu Glu Ala Val |
| | Arg Leu Phe Val Glu Trp Leu Lys Asn-NH₂ |
| 154 | Ala Gly Asp Gly Thr Phe Thr Ser Asp Leu Ser Lys Gln Leu Glu Glu Glu Ala Val |
| | Arg Leu Phe Val Glu Phe Leu Lys Asn-NH₂ |
| 155 | Ala Gly Asp Gly Thr Phe Thr Ser Asp Leu Ser Lys Gln Met Glu Glu Glu Ala Val |
| | Arg Leu Phe tButylgly Glu Trp Leu Lys Asn-NH₂ |
| 156 | Ala Gly Asp Gly Thr Phe Thr Ser Asp Leu Ser Lys Gln Leu Glu Glu Glu Ala Val |
| | Arg Leu Phe tButylgly Glu Phe Leu Lys Asn-NH₂ |
| 157 | Ala Gly Asp Gly Thr Phe Thr Ser Asp Leu Ser Lys Gln Met Glu Glu Glu Ala Val |
| | Arg Leu Phe Ile Asp Trp Leu Lys Asn-NH₂ |
| 158 | Ala Gly Asp Gly Thr Phe Thr Ser Asp Leu Ser Lys Gln Leu Glu Glu Glu Ala Val |
| | Arg Leu Phe Ile Asp Phe Leu Lys Asn-NH₂ |
| 159 | Ala Gly Asp Gly Thr Phe Thr Ser Asp Leu Ser Lys Gln Met Glu Glu Glu Ala Val |
| | Arg Leu Phe Ile Glu Ala Leu Lys Asn-NH₂ |
| 160 | Ala Gly Asp Gly Thr Phe Thr Ser Asp Leu Ser Lys Gln Leu Glu Glu Glu Ala Val |
| | Arg Leu Phe Ile Glu Ala Leu Lys Asn-NH₂ |
| 161 | Ala Gly Asp Gly Thr Phe Thr Ser Asp Leu Ser Lys Gln Met Glu Glu Glu Ala Val |
| | Arg Leu Phe Ile Glu Trp Ala Lys Asn-NH₂ |
| 162 | Ala Gly Asp Gly Thr Phe Thr Ser Asp Leu Ser Lys Gln Leu Glu Glu Glu Ala Val |
| | Arg Leu Phe Ile Glu Phe Ala Lys Asn-NH₂ |
| 163 | Ala Gly Asp Gly Thr Phe Thr Ser Asp Leu Ser Lys Gln Met Glu Glu Glu Ala Val |
| | Arg Leu Phe Ile Glu Trp Leu Ala Asn-NH₂ |
| 164 | Ala Gly Asp Gly Thr Phe Thr Ser Asp Leu Ser Lys Gln Leu Glu Glu Glu Ala Val |
| | Arg Leu Phe Ile Glu Phe Leu Ala Asn-NH₂ |
| 165 | Ala Gly Asp Gly Thr Phe Thr Ser Asp Leu Ser Lys Gln Met Glu Glu Glu Ala Val |
| | Arg Leu Phe Ile Glu Trp Leu Lys Ala-NH₂ |
| 166 | Ala Gly Asp Gly Thr Phe Thr Ser Asp Leu Ser Lys Gln Leu Glu Glu Glu Ala Val |
| | Arg Leu Phe Ile Glu Phe Leu Lys Ala-NH₂ |
| 167 | Ala Gly Glu Gly Thr Phe Thr Ser Asp Leu Ser Lys Gln Met Glu Glu Glu Ala Val |
| | Arg Leu Phe Ile Glu Trp Leu Lys Asn Gly Gly Pro Ser Ser Gly Ala Pro Pro Pro- |
| | NH₂ |
| 168 | His Gly Ala Gly Thr Phe Thr Ser Asp Leu Ser Lys Gln Leu Glu Glu Glu Ala Val |
| | Arg Leu Phe Ile Glu Phe Leu Lys Asn Gly Gly Pro Ser Ser Gly Ala Pro Pro Pro- |
| | NH₂ |
| 169 | His Gly Glu Ala Thr Phe Thr Ser Asp Leu Ser Lys Gln Met Glu Glu Glu Ala Val |
| | Arg Leu Phe Ile Glu Trp Leu Lys Asn Gly Gly Pro Ser Ser Gly Ala Pro Pro-NH₂ |
| 170 | His Gly Glu Gly Thr Phe Thr Ser Ala Leu Ser Lys Gln Met Glu Glu Glu Ala Val |
| | Arg Leu Phe Ile Glu Trp Leu Lys Asn Gly Gly Pro Ser Ser Gly Ala Pro-NH₂ |
| 171 | Ala Gly Glu Gly Thr Phe Thr Ser Asp Ala Ser Lys Gln Leu Glu Glu Glu Ala Val |
| | Arg Leu Phe Ile Glu Phe Leu Lys Asn Gly Gly Pro Ser Ser Gly Ala Pro-NH₂ |
| 172 | Ala Gly Glu Gly Thr Phe Thr Ser Asp Leu Ser Lys Gln Met Glu Glu Glu Ala Val |
| | Arg Leu Phe Ile Glu Trp Leu Lys Asn Gly Gly Pro Ser Ser Gly Ala-NH₂ |
| 173 | His Gly Ala Gly Thr Phe Thr Ser Asp Leu Ser Lys Gln Leu Glu Glu Glu Ala Val |
| | Arg Leu Phe Ile Glu Phe Leu Lys Asn Gly Gly Pro Ser Ser Gly Ala-NH₂ |
| 174 | His Gly Glu Ala Thr Phe Thr Ser Asp Leu Ser Lys Gln Met Glu Glu Glu Ala Val |
| | Arg Leu Phe Ile Glu Trp Leu Lys Asn Gly Gly Pro Ser Ser Gly-NH₂ |
| 175 | His Gly Glu Gly Thr Phe Thr Ser Ala Leu Ser Lys Gln Met Glu Glu Glu Ala Val |
| | Arg Leu Phe Ile Glu Trp Leu Lys Asn Gly Gly Pro Ser Ser-NH₂ |
| 176 | Ala Gly Glu Gly Thr Phe Thr Ser Asp Leu Ser Lys Gln Met Glu Glu Glu Ala Val |
| | Arg Leu Phe Ile Glu Trp Leu Lys Asn Gly Gly Pro Ser-NH₂ |
| 177 | His Gly Ala Gly Thr Phe Thr Ser Asp Leu Ser Lys Gln Leu Glu Glu Glu Ala Val |
| | Arg Leu Phe Ile Glu Phe Leu Lys Asn Gly Gly Pro Ser-NH₂ |
| 178 | His Gly Glu Ala Thr Phe Thr Ser Asp Leu Ser Lys Gln Met Glu Glu Glu Ala Val |
| | Arg Leu Phe Ile Glu Trp Leu Lys Asn Gly Gly Pro-NH₂ |
| 179 | His Gly Glu Gly Thr Phe Thr Ser Ala Leu Ser Lys Gln Leu Glu Glu Glu Ala Val |
| | Arg Leu Phe Ile Glu Phe Leu Lys Asn Gly Gly-NH₂ |
| 180 | Ala Gly Glu Gly Thr Phe Thr Ser Asp Leu Ser Lys Gln Leu Glu Glu Glu Ala Val |
| | Arg Leu Phe Ile Glu Phe Leu Lys Asn Gly-NH₂ |
| 181 | His Gly Ala Gly Thr Phe Thr Ser Asp Leu Ser Lys Gln Met Glu Glu Glu Ala Val |
| | Arg Leu Phe Ile Glu Trp Leu Lys Asn Gly Gly thioPro Ser Ser Gly Ala thioPro |
| | thioPro thioPro-NH₂ |
| 182 | His Gly Glu Ala Thr Phe Thr Ser Asp Leu Ser Lys Gln Met Glu Glu Glu Ala Val |
| | Arg Leu Phe Ile Glu Trp Leu Lys Asn Gly Gly Pro Ser Ser Gly Ala thioPro |
| | thioPro thioPro-NH₂ |
| 183 | His Gly Glu Gly Thr Phe Thr Ser Ala Leu Ser Lys Gln Met Glu Glu Glu Ala Val |
| | Arg Leu Phe Ile Glu Trp Leu Lys Asn Gly Gly NMeala Ser Ser Gly Ala NMeala |
| | NMeala-NH₂ |
| 184 | Ala Gly Glu Gly Thr Phe Thr Ser Asp Leu Ser Lys Gln Met Glu Glu Glu Ala Val |
| | Arg Leu Phe Ile Glu Trp Leu Lys Asn Gly Gly homoPro Ser Ser Gly Ala |
| | homoPro-NH₂ |
| 185 | His Gly Ala Gly Thr Phe Thr Ser Asp Leu Ser Lys Gln Met Glu Glu Glu Ala Val |
| | Arg Leu Phe Ile Glu Trp Leu Lys Asn Gly Gly Pro Ser Ser Gly Ala-NH₂ |
| 186 | His Gly Asp Ala Thr Phe Thr Ser Asp Leu Ser Lys Gln Met Glu Glu Glu Ala Val |
| | Arg Leu Phe Ile Glu Trp Leu Lys Asn Gly Gly-NH₂ |
| 187 | Ala Gly Glu Gly Thr Phe Thr Ser Asp Leu Ser Lys Gln Met Glu Glu Glu Ala Val |
| | Arg Leu Phe Ile Glu Trp Leu Lys Asn Gly Gly Pro Ser Ser Gly Ala Pro Pro Pro |
| | Ser-NH₂ |
| 188 | Ala Gly Ala Gly Thr Phe Thr Ser Asp Leu Ser Lys Gln Leu Glu Glu Glu Ala Val |
| | Arg Leu Phe Ile Glu Phe Leu Lys Asn Gly Gly Pro Ser Ser Gly Ala Pro Pro Pro |
| | Ser-NH₂ |

In one aspect, a bioactive peptide included in the formulation is a peptide in Pancreatic Polypeptide Family (PPF peptide). In another aspect, the peptide is a human pancreatic peptide polypeptide (human PP) or a species variant thereof. In another aspect, the peptide is a human NPY peptide or species variant thereof. See, e.g., WO2005077094 and Gehlert, Proc. Soc. Exp. Biol. Med. 1998; 218: 7-22. Formulations can also include analog, derivative, modified, chimeric and/or hybrid forms of PP and/or NPY peptides.

In still another aspect, the bioactive peptide is a peptide which lacks the first two amino acids of PYY (e.g., PYY(3-36) (See, e.g., Eberlein et al., Peptides 1989; 10: 797-803; Grandt et al., Regul. Pept. 1994; 51: 151-9) or is an analog thereof which has at least 50% sequence identity to PYY (3-36) over the entire length o PYY(3-36), and also comprise at least two PPF motifs including at least the N-terminal polyproline PPF motif and the C-terminal tail PPF motif. Additional PPF motifs can correspond to any motif of any of the PP family polypeptides, including PP, PYY and NPY. See, e.g., WO2005077094.

Additional PYY peptides that can be used in the compositions disclosed herein include any bioactive PYY peptide, PYY analog or PYY derivative known in the art such as those as described in International Patent Application Publication Nos. WO 02/47712 and WO 03/26591; and US Patent Application Publication No. 2002-141985. Particular examples of PYY peptides, PYY analogs and PYY derivatives that can be used in the compositions disclosed herein, include, but are not limited to those described in Table 2. Also included are other Y receptor family peptide agonists, particularly Y2, Y5, and putative Y7 receptor agonists and derivatives thereof. In one embodiment, the bioactive peptide is PYY₃₋₃₆.

TABLE 2
PYY Peptides, Analogs and Derivatives

| SEQ | |
|---|---|
| ID NO | Table 2 Sequence |
| 189 | Ala Pro Leu Glu Pro Val Tyr Pro Gly Asp Asn Ala Thr Pro Glu Gln Met |
| | Ala Gln Tyr Ala Ala Asp Leu Arg Arg Tyr Ile Asn Met Leu Thr Arg Pro |
| | Arg Tyr |
| 190 | Tyr Pro Ile Lys Pro Glu Ala Pro Gly Glu Asp Ala Ser Pro Glu Glu Leu |
| | Asn Arg Tyr Tyr Ala Ser Leu Arg His Tyr Leu Asn Leu Val Thr Arg Gln |
| | Arg Tyr |
| 191 | Ile Lys Pro Glu Ala Pro Gly Glu Asp Ala Ser Pro Glu Glu Leu Asn Arg |
| | Tyr Tyr Ala Ser Leu Arg His Tyr Leu Asn Leu Val Thr Arg Gln Arg Tyr |
| 192 | Tyr Pro Ser Lys Pro Asp Asn Pro Gly Glu Asp Ala Pro Ala Glu Asp Met |
| | Ala Arg Tyr Tyr Ser Ala Leu Arg His Tyr Ile Asn Leu Ile Thr Arg Gln Arg |
| | Tyr |
| 193 | Ser Lys Pro Asp Asn Pro Gly Glu Asp Ala Pro Ala Glu Asp Met Ala Arg |
| | Tyr Tyr Ser Ala Leu Arg His Tyr Ile Asn Leu Ile Thr Arg Gln Arg Tyr |
| 194 | Ala Ser Leu Arg His Tyr Leu Asn Leu Val Thr Arg Gln Arg Tyr |

In another aspect, the bioactive peptide comprises a human glucagon like peptide-1 (GLP-1) or species variants thereof, an analog, a derivative, modified, chimeric and/or hybrid form thereof. See, e.g., WO2005000892, WO2004022004, WO2005097175, WO 01/98331, WO 02/48192; US2003220243; US2004-053819; U.S. Pat. No. 5,981,488, U.S. Pat. No. 5,574,008, U.S. Pat. No. 5,512,549, and U.S. Pat. No. 5,705,483.

In additional embodiments, the bioactive peptide comprises a GLP-1 analog or GLP-1 derivative such as GLP-1 (7-37), GLP-1(7-36)NH₂, Gly⁸ GLP-1(7-37), Ser³⁴ GLP-1(7-37) Val⁸ GLP-1(7-37) and Val⁸ Glu²² GLP-1(7-37). Any bioactive GLP-1, GLP-1 analog or GLP-1 derivative known in the art can be used in the present formulations, including, but not limited to those described in WO 01/98331, WO 02/48192; US2003220243; US2004053819; U.S. Pat. No. 5,981,488; U.S. Pat. No. 5,574,008; U.S. Pat. No. 5,512,549; and U.S. Pat. No. 5,705,483. Examples of GLP-1 peptides that are suitable for use in the formulations disclosed herein are those described in US2003220243 by the following formulas:

Formula IV

(SEQ ID No. 244)

His-Xaa₈-Glu-Gly-Xaa₁₁-Xaa₁₂-Thr-Ser-
Asp-Xaa₁₆-Ser-Ser-Tyr-Leu-Glu-Xaa₂₂-Xaa₂₃-
Xaa₂₄-Ala-Xaa₂₆-Xaa₂₇-Phe-Ile-Ala-Xaa₃₁-
Leu-Xaa₃₃-Xaa₃₄-Xaa₃₅-Xaa₃₆-R

where:
Xaa₅ is Gly, Ala, Val, Leu, Ile, Ser, or Thr;
Xaa₁₁ is Asp, Glu, Arg, Thr, Ala, Lys, or His;
Xaa₁₂ is His, Trp, Phe, or Tyr;
Xaa₁₆ is Leu, Ser, Thr, Trp, His, Phe, Asp, Val, Glu, or Ala;
Xaa₂₂ is Gly, Asp, Glu, Gln, Asn, Lys, Arg, Cys, or Cysteic Acid;
Xaa₂₃ is His, Asp, Lys, Glu, or Gln;
Xaa₂₄ is Glu, His, Ala, or Lys;
Xaa₂₆ is Asp, Lys, Glu, or His;
Xaa₂₇ is Ala, Glu, His, Phe, Tyr, Trp, Arg, or Lys;
Xaa₃₁ is Ala, Glu, Asp, Ser, or His;
Xaa₃₃ is Asp, Arg, Val, Lys, Ala, Gly, or Glu;
Xaa₃₄ is Glu, Lys, or Asp;
Xaa₃₅ is Thr, Ser, Lys, Arg, Trp, Tyr, Phe, Asp, Gly, Pro, His, or Glu;
Xaa₃₆ is Arg, Glu, or His; and
R is: Lys, Arg, Thr, Ser, Glu, Asp, Trp, Tyr, Phe, His, —NH₂, Gly, Gly-Pro, or Gly-Pro-NH₂, or is deleted.

Formula V

(SEQ ID No. 245)

His-Xaa₈-Glu-Gly-Thr-Xaa₁₂-Thr-Ser-Asp-
Xaa₁₆-Ser-Ser-Tyr-Leu-Glu-Xaa₂₂-Xaa₂₃-
Ala-Ala-Xaa₂₆-Glu-Phe-Ile-Xaa₃₀-Trp-Leu-
Val-Lys-Xaa₃₅-Arg-R

where:
Xaa₈ is Gly, Ala, Val, Leu, Ile, Ser, or Thr;
Xaa₁₂ is His, Trp, Phe, or Tyr;
Xaa₁₆ is Leu, Ser, Thr, Trp, His, Phe, Asp, Val, Glu, or Ala;
Xaa₂₂ is Gly, Asp, Glu, Gln, Asn, Lys, Arg, Cys, or Cysteic Acid (3-Sulfoalanine);
Xaa₂₃ is His, Asp, Lys, Glu, or Gln;
Xaa₂₆ is: Asp, Lys, Glu, or His;
Xaa₃₀ is Ala, Glu, Asp, Ser, or His;
Xaa₃₅ is Thr, Ser, Lys, Arg, Trp, Tyr, Phe, Asp, Gly, Pro, His, or Glu; and
R is: Lys, Arg, Thr, Ser, Glu, Asp, Trp, Tyr, Phe, His, —NH₂, Gly, Gly-Pro, or Gly-Pro-NH₂, or is deleted.

Formula VI

(SEQ ID No. 246)

His-Xaa₈-Glu-Gly-Thr-Phe-Thr-Ser-Asp-Val-Ser-
Ser-Tyr-Leu-Glu-Xaa₂₂-Xaa₂₃-Ala-Ala-Lys-
Xaa₂₇-Phe-Ile-Xaa₃₀-Trp-Leu-Val-Lys-Gly-Arg-R

where:
Xaa₈ is Gly, Ala, Val, Leu, Ile, Ser, or Thr;
Xaa₂₂ is Gly, Asp, Glu, Gln, Asn, Lys, Arg, Cys, or Cysteic Acid (3-Sulfoalanine);
Xaa₂₃ is His, Asp, Lys, Glu, or Gln;
Xaa₂₇ is Ala, Glu, His, Phe, Tyr, Trp, Arg, or Lys
Xaa₃₀ is Ala, Glu, Asp, Ser, or His; and
R is: Lys, Arg, Thr, Ser, Glu, Asp, Trp, Tyr, Phe, His, —NH₂, Gly, Gly-Pro, or Gly-Pro-NH₂, or is deleted.

Formula VII

(SEQ ID No. 247)

Xaa₇-Xaa₈-Glu-Gly-Thr-Phe-Thr-Ser-Asp-Val-Ser-
Ser-Tyr-Leu-Glu-Xaa₂₂-Gln-Ala-Ala-Lys-Glu-
Phe-Ile-Ala-Trp-Leu-Val-Lys-Gly-Arg-R

where:
Xaa₇ is L-histidine, D-histidine, desamino-histidine, 2amino-histidine, β-hydroxy-histidine, homohistidine, α-fluoromethyl-histidine or α-methyl-histidine;
Xaa₈ is glycine, alanine, valine, leucine, isoleucine, serine or threonine;
Xaa₂₂ is aspartic acid, glutamic acid, glutamine, asparagine, lysine, arginine, cysteine, or cysteic acid; and
R is —NH₂ or Gly(OH).

Particular, but non-limiting examples of GLP1 peptides that can be use in the present compositions can be found in Table 3

TABLE 3
GLP-1 Peptides, Analogs and Derivatives

| SEQ | |
|---|---|
| ID NO | Table 3 Sequence |
| 195 | His Val Glu Gly Thr Phe Thr Ser Asp Val Ser Ser Tyr Leu Glu Glu Gln Ala Ala |
| | Lys Glu Phe Ile Ala Trp Leu Val Lys Gly Arg Gly |
| 196 | His Val Glu Gly Thr Phe Thr Ser Asp Val Ser Ser Tyr Leu Glu Asp Gln Ala Ala |
| | Lys Glu Phe Ile Ala Trp Leu Val Lys Gly Arg Gly |
| 197 | His Val Glu Gly Thr Phe Thr Ser Asp Val Ser Ser Tyr Leu Glu Arg Gln Ala Ala |
| | Lys Glu Phe Ile Ala Trp Leu Val Lys Gly Arg Gly |
| 198 | His Val Glu Gly Thr Phe Thr Ser Asp Val Ser Ser Tyr Leu Glu Lys Gln Ala Ala |
| | Lys Glu Phe Ile Ala Trp Leu Val Lys Gly Arg Gly |
| 199 | His Gly Glu Gly Thr Phe Thr Ser Asp Val Ser Ser Tyr Leu Glu Glu Gln Ala Ala |
| | Lys Glu Phe Ile Ala Trp Leu Val Lys Gly Arg Gly |
| 200 | His Gly Glu Gly Thr Phe Thr Ser Asp Val Ser Ser Tyr Leu Glu Asp Gln Ala Ala |
| | Lys Glu Phe Ile Ala Trp Leu Val Lys Gly Arg Gly |
| 201 | His Gly Glu Gly Thr Phe Thr Ser Asp Val Ser Ser Tyr Leu Glu Arg Gln Ala Ala |
| | Lys Glu Phe Ile Ala Trp Leu Val Lys Gly Arg Gly |
| 202 | His Gly Glu Gly Thr Phe Thr Ser Asp Val Ser Ser Tyr Leu Glu Lys Gln Ala Ala |
| | Lys Glu Phe Ile Ala Trp Leu Val Lys Gly Arg Gly |
| 203 | His Val Glu Gly Thr Phe Thr Ser Asp Val Ser Ser Tyr Leu Glu Gly Gln Ala Ala |
| | Lys Glu Phe Ile Glu Trp Leu Val Lys Gly Arg Gly |
| 204 | His Gly Glu Gly Thr Phe Thr Ser Asp Val Ser Ser Tyr Leu Glu Gly Gln Ala Ala |
| | Lys Glu Phe Ile Glu Trp Leu Val Lys Gly Arg Gly |
| 205 | His Val Glu Gly Thr Phe Thr Ser Asp Val Ser Ser Tyr Leu Glu Gly Gln Ala Ala |
| | Lys Glu Phe Ile Ala Trp Leu Val Lys Gly Arg His |
| 206 | His Gly Glu Gly Thr Phe Thr Ser Asp Val Ser Ser Tyr Leu Glu Gly Gln Ala Ala |
| | Lys Glu Phe Ile Ala Trp Leu Val Lys Gly Arg His |
| 207 | His Val Glu Gly Thr Phe Thr Ser Asp Val Ser Ser Tyr Leu Glu Glu Gln Ala Ala |
| | Lys Ala Phe Ile Ala Trp Leu Val Lys Gly Arg His |
| 208 | His Val Glu Gly Thr Phe Thr Ser Asp Val Ser Ser Tyr Leu Glu Lys Glu Ala Ala |
| | Lys Glu Phe Ile Ala Trp Leu Val Lys Gly Arg His |
| 209 | His Ala Glu Gly Thr Phe Thr Ser Asp Val Ser Ser Tyr Leu Glu Gly Gln Ala Ala |
| | Lys Glu Phe Ile Ala Trp Leu Val Lys Gly Arg Gly |
| 210 | His Ala Glu Gly Thr Phe Thr Ser Asp Val Ser Ser Tyr Leu Glu Glu Gln Ala Ala |
| | Lys Glu Phe Ile Ala Trp Leu Val Lys Gly Arg Gly |
| 211 | His Ala Glu Gly Thr Phe Thr Ser Asp Val Ser Ser Tyr Leu Glu Asp Gln Ala Ala |
| | Lys Glu Phe Ile Ala Trp Leu Val Lys Gly Arg Gly |
| 212 | His Ala Glu Gly Thr Phe Thr Ser Asp Val Ser Ser Tyr Leu Glu Arg Gln Ala Ala |
| | Lys Glu Phe Ile Ala Trp Leu Val Lys Gly Arg Gly |
| 213 | His Ala Glu Gly Thr Phe Thr Ser Asp Val Ser Ser Tyr Leu Glu Lys Gln Ala Ala |
| | Lys Glu Phe Ile Ala Trp Leu Val Lys Gly Arg Gly |
| 214 | His Ala Glu Gly Thr Phe Thr Ser Asp Val Ser Ser Tyr Leu Glu 3-sulfoAla Gln |
| | Ala Ala Lys Glu Phe Ile Ala Trp Leu Val Lys Gly Arg Gly |
| 215 | His Val Glu Gly Thr Phe Thr Ser Asp Val Ser Ser Tyr Leu Glu 3-sulfoAla Gln |
| | Ala Ala Lys Glu Phe Ile Ala Trp Leu Val Lys Gly Arg Gly |
| 216 | His Gly Glu Gly Thr Phe Thr Ser Asp Val Ser Ser Tyr Leu Glu 3-sulfoAla Gln |
| | Ala Ala Lys Glu Phe Ile Ala Trp Leu Val Lys Gly Arg Gly |
| 217 | His Ala Glu Gly Thr Phe Thr Ser Asp Val Ser Ser Tyr Leu Glu Glu Gln Ala Ala |
| | Lys Glu Phe Ile Ala Trp Leu Val Lys Gly Arg |
| 218 | His Ala Glu Gly Thr Phe Thr Ser Asp Val Ser Ser Tyr Leu Glu Asp Gln Ala Ala |
| | Lys Glu Phe Ile Ala Trp Leu Val Lys Gly Arg |
| 219 | His Ala Glu Gly Thr Phe Thr Ser Asp Val Ser Ser Tyr Leu Glu Arg Gln Ala Ala |
| | Lys Glu Phe Ile Ala Trp Leu Val Lys Gly Arg |
| 220 | His Ala Glu Gly Thr Phe Thr Ser Asp Val Ser Ser Tyr Leu Glu Lys Gln Ala Ala |
| | Lys Glu Phe Ile Ala Trp Leu Val Lys Gly Arg |
| 221 | His Ala Glu Gly Thr Phe Thr Ser Asp Val Ser Ser Tyr Leu Glu 3-sulfoAla Gln |
| | Ala Ala Lys Glu Phe Ile Ala Trp Leu Val Lys Gly Arg |
| 222 | His Val Glu Gly Thr Phe Thr Ser Asp Val Ser Ser Tyr Leu Glu Glu Gln Ala Ala |
| | Lys Glu Phe Ile Ala Trp Leu Val Lys Gly Arg |
| 223 | His Val Glu Gly Thr Phe Thr Ser Asp Val Ser Ser Tyr Leu Glu Asp Gln Ala Ala |
| | Lys Glu Phe Ile Ala Trp Leu Val Lys Gly Arg |
| 224 | His Val Glu Gly Thr Phe Thr Ser Asp Val Ser Ser Tyr Leu Glu Arg Gln Ala Ala |
| | Lys Glu Phe Ile Ala Trp Leu Val Lys Gly Arg |
| 225 | His Val Glu Gly Thr Phe Thr Ser Asp Val Ser Ser Tyr Leu Glu Lys Gln Ala Ala |
| | Lys Glu Phe Ile Ala Trp Leu Val Lys Gly Arg |
| 226 | His Val Glu Gly Thr Phe Thr Ser Asp Val Ser Ser Tyr Leu Glu 3-sulfoAla Gln |
| | Ala Ala Lys Glu Phe Ile Ala Trp Leu Val Lys Gly Arg |
| 227 | His Gly Glu Gly Thr Phe Thr Ser Asp Val Ser Ser Tyr Leu Glu Glu Gln Ala Ala |
| | Lys Glu Phe Ile Ala Trp Leu Val Lys Gly Arg |
| 228 | His Gly Glu Gly Thr Phe Thr Ser Asp Val Ser Ser Tyr Leu Glu Asp Gln Ala Ala |
| | Lys Glu Phe Ile Ala Trp Leu Val Lys Gly Arg |
| 229 | His Gly Glu Gly Thr Phe Thr Ser Asp Val Ser Ser Tyr Leu Glu Arg Gln Ala Ala |
| | Lys Glu Phe Ile Ala Trp Leu Val Lys Gly Arg |
| 230 | His Gly Glu Gly Thr Phe Thr Ser Asp Val Ser Ser Tyr Leu Glu Lys Gln Ala Ala |
| | Lys Glu Phe Ile Ala Trp Leu Val Lys Gly Arg |
| 231 | His Gly Glu Gly Thr Phe Thr Ser Asp Val Ser Ser Tyr Leu Glu 3-sulfoAla Gln |
| | Ala Ala Lys Glu Phe Ile Ala Trp Leu Val Lys Gly Arg |
| 232 | His Val Glu Gly Thr Phe Thr Ser Asp Val Ser Ser Tyr Leu Glu Gly Lys Ala Ala |
| | Lys Glu Phe Ile Ala Trp Leu Val Lys Gly Arg Gly |
| 233 | His Val Glu Gly Thr Phe Thr Ser Asp Val Ser Ser Tyr Leu Glu Gly Gln Ala Ala |
| | Lys Ala Phe Ile Ala Trp Leu Val Lys Gly Arg Gly |
| 234 | His Val Glu Gly Thr Phe Thr Ser Asp Val Ser Ser Tyr Leu Glu Gly Gln Ala Ala |
| | Lys Glu Phe Ile Glu Trp Leu Val Lys Gly Arg Gly |
| 235 | His Gly Glu Gly Thr Phe Thr Ser Asp Val Ser Ser Tyr Leu Glu Gly Gln Ala Ala |
| | Lys Glu Phe Ile Glu Trp Leu Val Lys Gly Arg Gly |
| 236 | His Val Glu Gly Thr Phe Thr Ser Asp Val Ser Ser Tyr Leu Glu Gly Gln Ala Ala |
| | Lys Glu Phe Ile Ala Trp Leu Val Lys His Arg Gly |
| 237 | His Val Glu Gly Thr Phe Thr Ser Asp Val Ser Ser Tyr Leu Glu Gly Gln Ala Ala |
| | Lys Glu Phe Ile Ala Trp Leu Val Lys Gly Arg His |
| 238 | His Val Glu Gly Thr Phe Thr Ser Asp Val Ser Ser Tyr Leu Glu Glu Lys Ala Ala |
| | Lys Glu Phe Ile Ala Trp Leu Val Lys Gly Arg Gly |
| 239 | His Val Glu Gly Thr Phe Thr Ser Asp Val Ser Ser Tyr Leu Glu Glu Glu Ala Ala |
| | Lys Glu Phe Ile Ala Trp Leu Val Lys Gly Arg Gly |
| 240 | His Val Glu Gly Thr Phe Thr Ser Asp Val Ser Ser Tyr Leu Glu Glu Gln Ala Ala |
| | Lys Ala Phe Ile Ala Trp Leu Val Lys Gly Arg Gly |
| 241 | His Val Glu Gly Thr Phe Thr Ser Asp Val Ser Ser Tyr Leu Glu Gly Gln Ala Ala |
| | Lys Glu Phe Ile Ala Trp Leu Val Gly Lys Arg Gly |
| 242 | His Val Glu Gly Thr Phe Thr Ser Asp Val Ser Ser Tyr Leu Glu Gly Gln Ala Ala |
| | Lys Glu Phe Ile Ala Trp Leu Val Lys Gly Arg His |
| 243 | His Gly Glu Gly Thr Phe Thr Ser Asp Val Ser Ser Tyr Leu Glu Gly Gln Ala Ala |
| | Lys Glu Phe Ile Ala Trp Leu Val Lys Gly Arg His |

In further embodiments, the bioactive peptide or protein of the compositions disclosed herein comprise amylin, amylin analogs and amylin derivatives. Any amylin, amylin analogs or amylin deriviatives known in the art can be used in the present compositions, including, but not limited to those disclosed in U.S. Pat. Nos. 6,610,824, 5,686,411, 5,580,953, 5,367,052 and 5,124,314. Examples of amylin peptides that may be used are described by the following formula:

Formula VIII

(SEQ ID NO. 248)

A₁-X-Asn-Thr-Ala-Thr-Y-Ala-Thr-Gln-Arg-Leu-B₁-Asn-
Phe-Leu-C₁-D₁-E₁-F₁-G₁-Asn-H₁-Gly-I₁-J₁-
Leu-K₁-L₁-Thr-M₁-Val-Gly-Ser-Asn-Thr-Tyr-Z,

where:

A₁ is Lys, Ala, Ser or hydrogen,

B₁ is Ala, Set or Thr;

C₁ is Val, Leu or Ile;

D₁ is His or Arg;

E₁ is Ser or Thr;

F₁ is Ser, Thr, Gln or Asn;

G₁ is Asn, Gln or His;

H₁ is Phe, Leu or Tyr;

I₁ is Ala or Pro;

J₁ is Ile, Val, Ala or Leu;

K₁ is Ser, Pro, Leu, Ile or Thr;

L₁ is Ser, Pro or Thr;

M₁ is Asn, Asp, or Gln;

X and Y are independently selected amino acid residues having side chains which are chemically bonded to each other to form an intramolecular linkage; and
Z is amino, alkylamino, dialkylamino, cycloalkylamino, arylamino, aralkylamino, alkyloxy, aryloxy or aralkyloxy. Particular, but non-limiting examples of amylin analogs and derivatives that can be used are presented in Table 4.

TABLE 4
Amylin analogs and derivatives

| SEQ | |
|---|---|
| ID NO | Table 4 Sequence |
| 249 | Lys Cys Asn Thr Ala Thr Cys Ala Thr Gln Arg Leu Ala Asn Phe Leu Val His Ser |
| | Ser Asn Asn Phe Gly Ala Ile Leu Ser Ser Thr Asn Val Gly Ser Asn Thr Tyr |
| 250 | Lys Cys Asn Thr Ala Thr Cys Ala Thr Gln Arg Leu Ala Asn Phe Leu Ile Arg Ser |
| | Ser Asn Asn Leu Gly Ala Ile Leu Ser Pro Thr Asn Val Gly Ser Asn Thr Tyr |
| 251 | Lys Cys Asn Thr Ala Thr Cys Ala Thr Gln Arg Leu Ala Asn Phe Leu Val Arg Thr |
| | Ser Asn Asn Leu Gly Ala Ile Leu Ser Pro Thr Asn Val Gly Ser Asn Thr Tyr |
| 252 | Lys Cys Asn Thr Ala Thr Cys Ala Thr Gln Arg Leu Ala Asn Phe Leu Val Arg Ser |
| | Ser Asn Asn Leu Gly Pro Val Leu Pro Pro Thr Asn Val Gly Ser Asn Thr Tyr |
| 253 | Lys Cys Asn Thr Ala Thr Cys Ala Thr Gln Arg Leu Ala Asn Phe Leu Val His Ser |
| | Asn Asn Asn Leu Gly Pro Val Leu Ser Pro Thr Asn Val Gly Ser Asn Thr Tyr |
| 254 | Lys Cys Asn Thr Ala Thr Cys Ala Thr Gln Arg Leu Thr Asn Phe Leu Val Arg Ser |
| | Ser His Asn Leu Gly Ala Ala Leu Leu Pro Thr Asp Val Gly Ser Asn Thr Tyr |
| 255 | Cys Asn Thr Ala Thr Cys Ala Thr Gln Arg Leu Ala Asn Phe Leu Val His Ser Ser |
| | Asn Asn Phe Gly Ala Ile Leu Ser Ser Thr Asn Val Gly Ser Asn Thr Tyr |
| 256 | Lys Cys Asn Thr Ala Thr Cys Ala Thr Gln Arg Leu Ala Asn Phe Leu Val His Ser |
| | Ser Asn Asn Phe Gly Ala Ile Leu Pro Ser Thr Asn Val Gly Ser Asn Thr Tyr |
| 257 | Lys Cys Asn Thr Ala Thr Cys Ala Thr Gln Arg Leu Ala Asn Phe Leu Val His Ser |
| | Ser Asn Asn Phe Gly Pro Ile Leu Pro Pro Thr Asn Val Gly Ser Asn Thr Tyr |
| 258 | Lys Cys Asn Thr Ala Thr Cys Ala Thr Gln Arg Leu Ala Asn Phe Leu Val Arg Ser |
| | Ser Asn Asn Phe Gly Pro Ile Leu Pro Ser Thr Asn Val Gly Ser Asn Thr Tyr |
| 259 | Cys Asn Thr Ala Thr Cys Ala Thr Gln Arg Leu Ala Asn Phe Leu Val His Arg Ser |
| | Asn Asn Phe Gly Pro Ile Leu Pro Ser Thr Asn Val Gly Ser Asn Thr Tyr |
| 260 | Lys Cys Asn Thr Ala Thr Cys Ala Thr Gln Arg Leu Ala Asn Phe Leu Val His Ser |
| | Ser Asn Asn Phe Gly Pro Val Leu Pro Pro Thr Asn Val Gly Ser Asn Thr Tyr |
| 261 | Lys Cys Asn Thr Ala Thr Cys Ala Thr Gln Arg Leu Ala Asn Phe Leu Val Arg Ser |
| | Ser Asn Asn Phe Gly Pro Ile Leu Pro Pro Thr Asn Val Gly Ser Asn Thr Tyr |
| 262 | Cys Asn Thr Ala Thr Cys Ala Thr Gln Arg Leu Ala Asn Phe Leu Val Arg Ser Ser |
| | Asn Asn Phe Gly Pro Ile Leu Pro Pro Ser Asn Val Gly Ser Asn Thr Tyr |
| 263 | Cys Asn Thr Ala Thr Cys Ala Thr Gln Arg Leu Ala Asn Phe Leu Val His Ser Ser |
| | Asn Asn Phe Gly Pro Ile Leu Pro Pro Ser Asn Val Gly Ser Asn Thr Tyr |
| 264 | Lys Cys Asn Thr Ala Thr Cys Ala Thr Gln Arg Leu Ala Asn Phe Leu Val His Ser |
| | Ser Asn Asn Leu Gly Pro Val Leu Pro Pro Thr Asn Val Gly Ser Asn Thr Tyr |
| 265 | Lys Cys Asn Thr Ala Thr Cys Ala Thr Gln Arg Leu Ala Asn Phe Leu Val His Ser |
| | Ser Asn Asn Leu Gly Pro Val Leu Pro Ser Thr Asn Val Gly Ser Asn Thr Tyr |
| 266 | Cys Asn Thr Ala Thr Cys Ala Thr Gln Arg Leu Ala Asn Phe Leu Val His Ser Ser |
| | Asn Asn Leu Gly Pro Val Leu Pro Ser Thr Asn Val Gly Ser Asn Thr Tyr |
| 267 | Lys Cys Asn Thr Ala Thr Cys Ala Thr Gln Arg Leu Ala Asn Phe Leu Val Arg Ser |
| | Ser Asn Asn Leu Gly Pro Val Leu Pro Ser Thr Asn Val Gly Ser Asn Thr Tyr |
| 268 | Lys Cys Asn Thr Ala Thr Cys Ala Thr Gln Arg Leu Ala Asn Phe Leu Val Arg Ser |
| | Ser Asn Asn Leu Gly Pro Ile Leu Pro Pro Thr Asn Val Gly Ser Asn Thr Tyr |
| 269 | Lys Cys Asn Thr Ala Thr Cys Ala Thr Gln Arg Leu Ala Asn Phe Leu Val Arg Ser |
| | Ser Asn Asn Leu Gly Pro Ile Leu Pro Ser Thr Asn Val Gly Ser Asn Thr Tyr |
| 270 | Lys Cys Asn Thr Ala Thr Cys Ala Thr Gln Arg Leu Ala Asn Phe Leu Ile His Ser |
| | Ser Asn Asn Leu Gly Pro Ile Leu Pro Pro Thr Asn Val Gly Ser Asn Thr Tyr |
| 271 | Lys Cys Asn Thr Ala Thr Cys Ala Thr Gln Arg Leu Ala Asn Phe Leu Val Ile Ser |
| | Ser Asn Asn Phe Gly Pro Ile Leu Pro Pro Thr Asn Val Gly Ser Asn Thr Tyr |
| 272 | Cys Asn Thr Ala Thr Cys Ala Thr Gln Arg Leu Ala Asn Phe Leu Ile His Ser Ser |
| | Asn Asn Leu Gly Pro Ile Leu Pro Pro Thr Asn Val Gly Ser Asn Thr Tyr |
| 273 | Lys Cys Asn Thr Ala Thr Cys Ala Thr Gln Arg Leu Ala Asn Phe Leu Ile Arg Ser |
| | Ser Asn Asn Leu Gly Ala Ile Leu Ser Ser Thr Asn Val Gly Ser Asn Thr Tyr |
| 274 | Lys Cys Asn Thr Ala Thr Cys Ala Thr Gln Arg Leu Ala Asn Phe Leu Ile Arg Ser |
| | Ser Asn Asn Leu Gly Ala Val Leu Ser Pro Thr Asn ValGly Ser Asn Thr Tyr |
| 275 | Lys Cys Asn Thr Ala Thr Cys Ala Thr Gln Arg Leu Ala Asn Phe Leu Ile Arg Ser |
| | Ser Asn Asn Leu Gly Pro Val Leu Pro Pro Thr Asn Val Gly Ser Asn Thr Tyr |
| 276 | Lys Cys Asn Thr Ala Thr Cys Ala Thr Gln Arg Leu Thr Asn Phe Leu Val His Ser |
| | Ser His Asn Leu Gly Ala Ala Leu Leu Pro Thr Asp Val Gly Ser Asn Thr Tyr |
| 277 | Lys Cys Asn Thr Ala Thr Cys Ala Thr Gln Arg Leu Thr Asn Phe Leu Val His Ser |
| | Ser His Asn Leu Gly Ala Ala Leu Ser Pro Thr Asp Val Gly Ser Asn Thr Tyr |
| 278 | Cys Asn Thr Ala Thr Cys Ala Thr Gln Arg Leu Thr Asn Phe Leu Val His Ser Ser |
| | His Asn Leu Gly Ala Val Leu Pro Ser Thr Asp Val Gly Ser Asn Thr Tyr |
| 279 | Lys Cys Asn Thr Ala Thr Cys Ala Thr Gln Arg Leu Thr Asn Phe Leu Val Arg Ser |
| | Ser His Asn Leu Gly Ala Ala Leu Ser Pro Thr Asp Val Gly Ser Asn Thr Tyr |
| 280 | Lys Cys Asn Thr Ala Thr Cys Ala Thr Gln Arg Leu Thr Asn Phe Leu Val Arg Ser |
| | Ser His Asn Leu Gly Ala Ile Leu Pro Pro Thr Asp Val Gly Ser Asn Thr Tyr |
| 281 | Lys Cys Asn Thr Ala Thr Cys Ala Thr Gln Arg Leu Thr Asn Phe Leu Val Arg Ser |
| | Ser His Asn Leu Gly Pro Ala Leu Pro Pro Thr Asp Val Gly Ser Asn Thr Tyr |
| 282 | Lys Asp Asn Thr Ala Thr Lys Ala Thr Gln Arg Leu Ala Asn Phe Leu Val His Ser |
| | Ser Asn Asn Phe Gly Ala Ile Leu Ser Ser Thr Asn Val Gly Ser Asn Thr Tyr |
| 283 | Ala Cys Asn Thr Ala Thr Cys Ala Thr Gln Arg Leu Ala Asn Phe Leu Val His Ser |
| | Ser Asn Asn Phe Gly Ala Ile Leu Ser Ser Thr Asn Val Gly Ser Asn Thr Tyr |
| 284 | Ser Cys Asn Thr Ala Thr Cys Ala Thr Gln Arg Leu Ala Asn Phe Leu Val His Ser |
| | Ser Asn Asn Phe Gly Ala Ile Leu Ser Ser Thr Asn Val Gly Ser Asn Thr Tyr |
| 285 | Lys Cys Asn Thr Ala Thr Cys Ala Thr Gln Arg Leu Ala Asn Phe Leu Val His Ser |
| | Ser Asn Asn Phe Gly Ala Ile Leu Ser Pro Thr Asn Val Gly Ser Asn Thr Tyr |
| 286 | Lys Cys Asn Thr Ala Thr Cys Ala Thr Gln Arg Leu Ala Asn Phe Leu Val His Ser |
| | Ser Asn Asn Phe Gly Pro Ile Leu Pro Ser Thr Asn Val Gly Ser Asn Thr Tyr |
| 287 | Cys Asn Thr Ala Thr Cys Ala Thr Gln Arg Leu Ala Asn Phe Leu Val His Ser Ser |
| | Asn Asn Phe Gly Pro Ile Leu Pro Ser Thr Asn Val Gly Ser Asn Thr Tyr |
| 288 | Cys Asn Thr Ala Thr Cys Ala Thr Gln Arg Leu Ala Asn Phe Leu Val His Ser Ser |
| | Asn Asn Phe Gly Pro Val Leu Pro Pro Ser Asn Val Gly Ser Asn Thr Tyr |
| 289 | Lys Cys Asn Thr Ala Thr Cys Ala Thr Gln Arg Leu Ala Asn Phe Leu Val His Ser |
| | Ser Asn Asn Phe Gly Ala Ile Leu Ser Ser Thr Asn Val Gly Ser Asn Thr Tyr |
| 290 | Lys Cys Asn Thr Ala Thr Cys Ala Thr Gln Arg Leu Ala Asn Phe Leu Val His Ser |
| | Ser Asn Asn Phe Gly Pro Ile Leu Pro Pro Thr Asn Val Gly Ser Asn Thr Tyr |
| 291 | Lys Cys Asn Thr Ala Thr Cys Val Leu Gly Arg Leu Ser Gln Glu Leu His Arg Leu |
| | Gln Thr Tyr Pro Arg Thr Asn Thr Gly Ser Asn Thr Tyr NH₂ |
| 292 | Cys Ser Asn Leu Ser Thr Cys Val Leu Gly Arg Leu Ser Gln Glu Leu His Arg Leu |
| | Gln Thr Tyr Pro Arg Thr Asn Thr Gly Ser Ans Thr Tyr NH₂ |

As discussed above, included in the compositions and methods disclosed herein are analogs and derivatives of bioactive peptides or proteins that have undergone one or more amino acid substitutions, additions or deletions. In one embodiment, the analog or derivative has undergone not more than 10 amino acid substitutions, deletions and/or additions. In another embodiment, the analog or derivative has undergone not more than 5 amino acid substitutions, deletions and/or additions.

It is recognized in the art that modifications in the amino acid sequence of a peptide, polypeptide, or protein can result in equivalent, or possibly improved, second generation peptides, etc., that display equivalent or superior functional characteristics when compared to the original amino acid sequence. Alterations can include amino acid insertions, deletions, substitutions, truncations, fusions, shuffling of subunit sequences, and the like.

One factor that can be considered in making such changes is the hydropathic index of amino acids. The importance of the hydropathic amino acid index in conferring interactive biological function on a protein has been discussed by Kyte and Doolittle (J. Mol. Biol., 157: 105-132, 1982). It is accepted that the relative hydropathic character of amino acids contributes to the secondary structure of the resultant protein.

Based on its hydrophobicity and charge characteristics, each amino acid has been assigned a hydropathic index as follows: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate/glutamine/aspartate/asparagine (−3.5); lysine (−3.9); and arginine (−4.5).

As is known in the art, certain amino acids in a peptide or protein can be substituted for other amino acids having a similar hydropathic index or score and produce a resultant peptide or protein having similar biological activity, i.e., which still retains biological functionality. In making such changes, it is preferable that amino acids having hydropathic indices within ±2 are substituted for one another. More preferred substitutions are those wherein the amino acids have hydropathic indices within ±1. Most preferred substitutions are those wherein the amino acids have hydropathic indices within ±0.5.

Like amino acids can also be substituted on the basis of hydrophilicity. U.S. Pat. No. 4,554,101 discloses that the greatest local average hydrophilicity of a protein, as governed by the hydrophilicity of its adjacent amino acids, correlates with a biological property of the protein. The following hydrophilicity values have been assigned to amino acids: arginine/lysine (+3.0); aspartate/glutamate (+3.0±1); serine (+0.3); asparagine/glutamine (+0.2); glycine (0); threonine (−0.4); proline (−0.5±1); alanine/histidine (−0.5); cysteine (−1.0); methionine (−1.3); valine (−1.5); leucine/isoleucine (−1.8); tyrosine (−2.3); phenylalanine (−2.5); and tryptophan (−3.4). Thus, one amino acid in a peptide, polypeptide, or protein can be substituted by another amino acid having a similar hydrophilicity score and still produce a resultant protein having similar biological activity, i.e., still retaining correct biological function. In making such changes, amino acids having hydrophilicity values within ±2 are preferably substituted for one another, those within ±1 are more preferred, and those within ±0.5 are most preferred.

As outlined above, amino acid substitutions in the bioactive peptides and proteins for use in the compositions and methods disclosed herein can be based on the relative similarity of the amino acid side-chain substituents, for example, their hydrophobicity, hydrophilicity, charge, size, etc. Exemplary substitutions that take various of the foregoing characteristics into consideration in order to produce conservative amino acid changes resulting in silent changes can be selected from other members of the class to which the naturally occurring amino acid belongs. Amino acids can be divided into the following four groups: (1) acidic amino acids; (2) basic amino acids; (3) neutral polar amino acids; and (4) neutral non-polar amino acids. Representative amino acids within these various groups include, but are not limited to: (1) acidic (negatively charged) amino acids such as aspartic acid and glutamic acid; (2) basic (positively charged) amino acids such as arginine, histidine, and lysine; (3) neutral polar amino acids such as glycine, serine, threonine, cysteine, cystine, tyrosine, asparagine, and glutamine; and (4) neutral non-polar amino acids such as alanine, leucine, isoleucine, valine, proline, phenylalanine, tryptophan, and methionine. It should be noted that changes which are not expected to be advantageous can also be useful if these result in the production of functional sequences.

Also included within the scope of the bioactive peptides and proteins that can be used in the present compositions are conjugates of the above referenced proteins, peptides and peptide analogs, e.g., chemically modified with or linked to at least one molecular weight enhancing compound known in the art such as polyethylene glycol, and chemically modified equivalents of such proteins, peptides, analogs, or conjugates. The polyethylene glycol polymers may have molecular weights between about 500 Da and 20,000 Da. Preferred conjugates include those described in International Patent Publication No. WO 00/66629, which is herein incorporated by reference in its entirety. In one embodiment, the bioactive peptides and proteins of the invention have a molecular weight up to about 100,000 Da, in another embodiment up to about 25,000 Da, while in still another embodiment up to about 5,000 Da.

In certain aspects of the invention, bioactive peptides are used in combination. Therefore, in one aspect, pre-lyophilization formulations include more than one bioactive peptide (e.g., two or more bioactive peptides) or include a bioactive peptide and one or more organic molecule(s) which have bioactive properties. In certain aspects, the bioactive property of the organic molecule(s) is to potentiate the activity of the bioactive molecule. For example, the organic molecule can include a DPP-IV inhibitor which increases the resistance of the bioactive peptide to DPP-IV cleavage when the peptide is administered to a subject.

Peptides can be prepared using standard solid-phase peptide synthesis techniques (see, e.g., U.S. Pat. No. 6,610,824, U.S. Pat. No. 5,686,411 and U.S. Pat. No. 6,610,824.), by recombinant techniques, by chemical ligation or other methods known in the art.

In certain aspects, peptides are provided as salts. Such salts include salts prepared with organic and inorganic acids, for example, HCl, HBr, H₂SO₄, H₃PO₄, trifluoroacetic acid, acetic acid, formic acid, methane-sulfonic acid, toluenesulfonic acid, maleic acid, fumaric acid and camphorsulfonic acid. Salts prepared with bases include ammonium salts, alkali metal salts, e.g. sodium and potassium salts, and alkali earth salts, e.g. calcium and magnesium salts. Acetate, hydrochloride, and trifluoroacetate salts are preferred. The salts may be formed by conventional means, as by reacting the free acid or base forms of the peptide with one or more equivalents of the appropriate base or acid in a solvent or medium in which the salt is insoluble, or in a solvent such as water which is then removed in vacuo or by freeze-drying or by exchanging the ions of an existing salt for another ion on a suitable ion exchange resin.

However, in certain aspects, the bioactive peptide is not provided as a salt. In further aspects, the pre-lyophilization formulation excludes salts.

In one embodiment, pre-lyophilization formulations according to the invention comprise a bioactive peptide and a solids content of at least about 20% w/w. The percent of solids contributed by the bioactive peptide can vary with the bioactive peptide and the intended use of the formulation after it is lyophilized and reconstituted. In one aspect, the peptide comprises about 0.05-5% w/w of the formulation. In another aspect, the peptide comprises about 0.75-1.5% w/w of the formulation. For example, the peptide can contribute from about 0.5-10%, or from about 0.5-6%, or about 0.5-3% of the solids content of the formulation. In one aspect, the bioactive peptide comprises 1.5% w/w of the formulation. In a further aspect, the peptide comprises 3.0% w/w of the formulation. In still another aspect, the peptide comprises 5.0% w/w of the formulation. In certain aspects, the peptide can be provided at a concentration which rages from about 1 mg/ml to about 10 mg/ml. For example, the peptide can be provided at a concentration of 3 mg/ml, 6 mg/ml or 10 mg/ml.

In certain embodiments, at least about 80% of the solids content is contributed to by a molecule which comprises a hydrophilic portion and a hydrophobic portion. In one aspect, the molecule can form an inclusion complex to shield a hydrophobic or lipophilic molecule from a hydrophilic environment, e.g., such as an aqueous solution. In another aspect, the molecule is used to dissolve the hydrophobic molecule in an aqueous solution. In one aspect, the molecule comprising the hydrophilic portion and the hydrophilic portion comprise a cyclic oligosaccharide, for example, such as a cyclodextrin.

Cyclodextrins can be neutral or charged, native (cyclodextrins α, β, γ, δ, ε), branched or polymerized, and in certain aspects, can be chemically modified, for example, by substitution of one or more hydroxypropyls by groups such as alkyls, aryls, arylalkyls, glycosidics, or by etherification, esterification with alcohols or aliphatic acids. Among the above groups, particular preference is given to those chosen from hydroxypropyl, methyl m, sulfobutylether groups. In certain aspects, cyclodextrins comprise six, seven, or eight glucopyranose units.

Suitable cyclodextrins according to aspects of the invention include α-cyclodextrin, β-cyclodextrin, and γ-cylcodextrin. For example, suitable α-cyclodextrins include, but are not limited to, hydroxypropyl-α-cyclodextrin, and hydroxyethyl-α-cyclodextrin. Suitable β-cyclodextrins include, but are not limited to, hydroxypropyl-β-cyclodextrin (e.g., such as 2-hydroxypropyl cyclodextrin), carboxymethyl-β-cyclodextrin, dihydroxypropyl-β-cyclodextrin, hydroxyethyl-β-cyclodextrin, 2,6-di-O-methyl-β-cyclodextrin, methyl-β-cyclodextrin, randomly methylated cylcodextrin, and sulfated-β-cyclodextrin. Suitable γ-cyclodextrins which may be used in are hydroxypropyl-γ-cyclodextrin, dihydroxypropyl-γ-cyclodextrin, hydroxyethyl-γ-cyclodextrin, and sulfated-γ-cyclodextrin.

In certain other embodiments, the pre-lyophilization formulation comprises a bioactive peptide, a lipid component, and an amount of a cyclic oligosaccharide to solubilize the lipid component. For example, the lipid component can be one which enhances the passage of the bioactive peptide through a mucosal lining, or through cell membranes more generally.

Suitable lipid components include, but are not limited to: liposomes (which may be charged or uncharged), long chain fatty acids, including, but not limited to unsaturated fatty acids, such as oleic acid, linoleic acid, monoolein, and the like, medium chain (C6 to C12) fatty acids, monoglycerides, and glycolipids, including, but not limited to short-chain sphingolipids (e.g., a short-chain glycosphingolipid or a short-chain sphingomyelin). Lipid components can also include N-[1-(2,3-dioleyloxy)propyl]N,N,N-trimethylammonium chloride (DOTMA), [N,N,N′,N′-tetramethyl-N,N-bis(2 hydroxyethyl)-2,3-di(oleoyloxy)-1,4-butanediammonium iodide] (Promega Madison, Wis., USA), dioctadecylamidoglycyl spermine (Promega Madison, Wis., USA), N-[1-(2,3-Dioleoyloxy)]N,N,N-trimethylammonium propane methylsulfate (DOTAP), N-[1-(2,3-dioleyloxy)propyl]N,N,N-trimethylammonium chloride, 1,2-dimyristyloxypropyl-3-dimethyl-hydroxy ethyl ammonium bromide (DMRIE), dimyristoleoyl phosphonomethyl trimethyl amn1onium (DMPTA); 1,2-dioleoyl-sn-glycero-3-phosphoethanolamine-N-(7-nitro-2-1,3-benzoxadiazol-4-yl), 1,2-dioleoyl-3 trimethylammonium-propane chloride, 1,2 dioleoyl-sn-glycero-3-phosphoethanolamine, and 1,3-dioleoyloxy-2-(6-carboxyspermyl)propylamide (DOSPER), and the like. Lipid components can optionally be provided as salts.

In one aspect, the lipid component comprises a phospholipid. In another aspect, the lipid component comprises 1,2-dimyristoylamido-1,2-deoxyphosphatidylcholine (“DDPC”).

In still further aspects, the phospholipid forms a lipid-complex or liposome complex with the bioactive peptide(s).

In certain aspects, the mass ratio of bioactive peptide to lipid component is less than 1:1; however, in other aspects, the ratio of bioactive peptide to lipid component is 1:1 or greater than 1:1. In still further aspects, the lipid component comprises about 0.5%, 1% or 2% w/w or more of the pre-lyophilization formulation. In certain other aspects, the mole ratio of the bioactive peptide to the molecule which comprises a hydrophilic portion and hydrophobic or lipophilic portion (e.g., cyclodextrin) comprises less than 1:1.

In one embodiment, the pre-lyophilization formulation comprises one or more buffer components, such that after lyophilization, lyophilized formulations can be reconstituted in a ready-to-use (e.g., ready-to-treat) form by the addition of water (e.g., such as sterile, non-pyrogenic water). Buffer component(s) can vary and can be selected to provide a suitable pH (e.g., from about 3-7) that will maximize the stability and activity (e.g., therapeutic effectiveness) of a bioactive peptide. In one aspect, the buffer component comprises tartrate. In certain embodiments, the buffer may be acetate, phosphate, citrate, glutamate, succinate (sodium or potassium), histidine, phosphate (sodium or potassium), Tris (tris (hydroxymethyl)aminomethane), diethanolamine, and the like.

Methods for calculating the buffering capacity (buffer value) of a buffer at a particular concentration and pH are well known in the art and can be determined by the skilled artisan without undue experimentation. In certain aspect, for example, where the formulation includes a polyamino acid, buffer components are selected which contain neutral and mono-anionic net charges. Examples of suitable buffers include, but are not limited to acetic acid, ε-aminocaproic acid, and glutamic acid.

In another embodiment, the pre-lyophilization formulation comprises a chelating component, such as EDTA or EGTA.

In a further embodiment, the pre-lyophilization formulation comprises a preservative component. Suitable preservatives include, but are not limited to: m-cresol, parabens (e.g., 0.18% methylparaben and 0.02% propylparaben), benzalkonium chloride (BAK), potassium sorbate, chlorhexidine acetate, chloroscresol and polyhexamine gluconate. However, in one aspect, the pre-lyophilization excludes BAK and includes parabens (e.g., a mixture of methylparaben and propylparaben). In one particular aspect, when the bioactive peptide includes an exendin, exendin analog, derivative, modified, chimeric or hybrid form thereof, the preservative excludes BAK and includes parabens. In additional aspects, the pre-lyophilization formulation excludes a preservative.

Tonicifying agents that may be used, include, but are not limited to, sodium chloride, mannitol, sucrose, and glucose. However, any tonicifying agent known in the art, and for example, which can be used to prevent mucosal irritation, can be used. In certain aspects, the tonicifying agent excludes sodium chloride and/or saccharides, disaccharides, and polyols.

Exemplary viscosity-increasing and bioadhesive agents that may be used in the compositions disclosed herein, include, but are not limited to, cellulose derivatives (e.g., hydroxypropyl cellulose, hydroxypropyl methylcellulose or methylcellulose of average molecular weight between 10 and 1,500 kDa), starch, gums, carbomers, and polycarbophil. However, any viscosity-increasing or bioadhesive agents known in the art to afford a higher viscosity or to increase the residence time of the pharmaceutical composition at the absorption site may be used.

Additional components which can be included comprise ionic and non-ionic (amphoteric) surfactants (e.g., polysorbates, cremophores, etc), bulking agents (e.g., a cyclodextrin, polyethylene glycol, and the like, and optionally, excluding saccharides, disaccharides, and polyols).

Suitable surfactants, which can be used, include but are not limited to: anionic surfactants such as salts of fatty acids, e.g., sodium lauryl sulphate and other sulphate salts of fatty acids; cationic surfactants, such as alkylamines; and nonionic surfactants, such as polysorbates and poloxaniers; as well as aliphatic monohydric alcohols of 5 to 25 carbon atoms such as decanol, lauryl alcohol, myristyl alcohol, palmityl alcohol, linolenyl alcohol and oleyl alcohol; other types of fatty acids of 5 to 30 carbon atoms such as oleic acid, stearic acid, linoleic acid, palmitic acid, myristic acid, lauric acid and capric acid and their esters. Additional surfactants include polysorbate 20 (Tween 20), polsorbate 80 (Tween 80), polyethylene glycol (PEG), cetyl alcohol, polyvinylpyrolidone (PVP), polyvinyl alcohol (PVA), lanolin alcohol, sorbitan monooleate, a cremophore, and didecanoyl phosphatidylcholine (DDPC), sodium cholate, sodium glycocholate, sodium glycodeoxycholate, taurodeoxycholate, sodium deoxycholate, sodium lithocholate chenocholate, chenodeoxycholate, ursocholate, ursodeoxy-cholate, hyodeoxycholate, dehydrocholate, glycochenocholate, taurochenocholate, and taurochenodeoxycholate and sodium dodecyl sulfate. Additional surfactants include sorbitan trioleate, soya lecithin, and oleic acid.

Formulations can additionally, or alternatively, include a polyamino acid. In one aspect, the permeation enhancer comprises a cationic polyamino acid. Suitable cationic polyamino acids include polymers of basic amino acids, such as histidine, arginine, and lysine, which are protonated in a neutral or acidic pH environment and are thus cationic. The molecular weight of such polymers, e.g., poly-L-histidine, poly-L-arginine, poly-L-lysine, or copolymers thereof, are generally between about 10 and about 300 kDa. In another embodiment, the polymers have an average molecular weight of between about 100 kDa and about 200 kDa. In still a further embodiment, the polymers have an average molecular weight between about 140 kDa and about 100 kDa, while in yet another embodiment the polymers have an average molecular weight of between about 140 kDa and about 500 kDa. In one particular embodiment, the cationic polyamino acid of the composition is poly-L-arginine hydrochloride with an average molecular weight of about 141 kDa. Methods for formulating bioactive peptides comprising cationic polyamino acids are described in, e.g., WO2005117584.

In certain aspects, formulations including bioactive peptides can include chitosan. As used herein, the term “chitosan” include all derivatives of chitin, or poly-N-acetyl-D-glucosamine, including all polyglucosamines and oligomers of glucosamine materials of different molecular weights, in which the greater proportion of the N-acetyl groups have been removed through hydrolysis (deacetylation). In one aspect, the degree of deacetylation, which represents the proportion of N-acetyl groups which have been removed through deacetylation, is in the range 40-97%, more preferably in the range 60-96% and most preferably be in the range 70-95%. In certain aspects, the chitosan component of the formulation has a molecular weight in the range of about 10,000 to 1,000,000 Da, in the range of about 15,000 to 750,000 Da, or in the range of about 20,000 to 500,000 Da. Salts of chitosan and chitosan derivatives are also encompassed in the scope of the invention and include, but are not limited to esters, ethers or other derivatives formed by bonding acyl and/or alkyl groups with the hydroxyl groups, but not the amino groups of chitosan. Examples include O-alkyl ethers of chitosan and O-acyl esters of chitosan. Modified chitosans, such as those conjugated to polyethylene glycol may also be used. See, e.g., as described in WO2005056008.

Formulations can also include bile salts and derivatives thereof as described, e.g., in U.S. Pat. No. 4,746,508.

Dimethyl sulfoxide (DMSO) can also be used in still other embodiments.

Other additional components can include excipients which the Federal Drug Administration (FDA) designates as ‘Generally Regarded as Safe’ (GRAS).

However, in certain aspects, the pre-lyophilization formulation excludes any polymers other than those contributed to the formulation by the peptide (e.g., a polymer conjugated to or fused to the peptide) or the cyclic oligosaccharide.

In one embodiment, the pre-lyophilization formulation comprises a bioactive peptide, a molecule comprising a hydrophilic portion and a hydrophobic or lipophilic portion, such as a cyclic oligosaccharide (e.g., cyclodextrin), a buffer component, a phospholipid component (e.g., DDPC), a chelating agent (e.g., such as EDTA), a preservative (e.g., parabens) and optionally, gelatin. In one aspect, the pre-lyophilization formulation consists essentially of a bioactive peptide, a molecule comprising a hydrophilic portion and a hydrophobic or lipophilic portion, such as a cyclic oligosaccharide (e.g., cyclodextrin), a buffer component, a phospholipid component (e.g., DDPC), a chelating agent (e.g., such as EDTA), and optionally, gelatin. As used herein, in one aspect, “consisting essentially of” excludes saccharides, disaccharides, polyols, solvents, and any polymers other than those contributed to the formulation by the peptide (e.g., a polymer conjugated to or fused to the peptide) or the molecule comprising a hydrophilic portion and a hydrophobic or lipophilic portion, and optionally, excludes surfactants (with the exception of the phospholipid component) and free amino acids. In another aspect, “consisting essentially of” excludes polyethylene glycol (except if the molecule is conjugated to the peptide), PVP or starch, monosaccharides, disaccharides, a polyhydroxy alcohol and/or free amino acids. In still other aspects, the pre-lyophilization formulation excludes non-cyclic polysaccharides, unless such polysaccharides are conjugated to the bioactive peptide.

In certain additional aspects, components which are excluded from the pre-lyophilization formulation can be added when reconstituting a lyophilized composition formed by lyophilizing the pre-lyophilization formulation.

In one embodiment, the invention further provides a kit comprising a pre-lyophilization formulation and components suitable for reconstituting a lyophilized composition formed by lyophilizing the pre-lyophilization formulation. In one aspect, the component comprises water (e.g., sterile, pyrogen-free water). In certain aspects, the component is a component excluded from the pre-lyophilization formulation. However, in other aspects, the component excluded from the pre-lyophilization formulation is also excluded from the reconstituted formulation.

Embodiments of the invention also provide stable lyophilized formulations, since a lyophilized pre-lyophilization formulation can be stored as a lyophilized composition. As discussed above, a “stable lyophilized composition” is one in which the active ingredient (e.g., such as a bioactive peptide) substantially retains its physical stability, chemical stability and/or biological activity upon storage. In one aspect, stable lyophilized compositions are those which retain biological activity (e.g., therapeutic activity) for at least about 1 month, at least about 2 months, at least about 3 months, at least about 4 months, at least about 5 months, at least about 6 months, at least about a year, at least about 2 years, or at least about five years. In certain aspects, the stable lyophilized composition retains biological activity and/or at least one therapeutic activity at least about 1 month, at least about 2 months, at least about 3 months, at least about 4 months, at least about 5 months, at least about 6 months, and at least about a year, at least about 2 years, or at least about five years at temperatures of at least about 20° C., e.g., 21° C., 22° C., 23° C., 24° C., or about 25° C.

Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 1993; 10:29-90. Stability can be measured at a selected temperature for a selected time period. In certain aspects, the purity of the peptide is also monitored, e.g., by SCX-HPLC, and at least about 95%, at least about 99%, and up to 100% of the theoretical content of the bioactive peptide can be reconstituted upon reconstituting the lyophilized composition.

Physical stability can be monitored by assessing aggregation, precipitation and/or denaturation of the peptide by a variety of methods, e.g., upon visual examination of color and/or clarity, or as measured by UV light scattering, size exclusion chromatography (SEC) and dynamic light scattering. Changes in conformation can be evaluated by methods known in the art, e.g., fluorescence spectroscopy or by FTIR spectroscopy.

Chemical stability can be assessed by detecting and quantifying chemically altered forms of the protein. Degradation processes that often alter the protein chemical structure include hydrolysis or clipping (evaluated by methods such as size exclusion chromatography and SDS-PAGE), oxidation (evaluated by methods such as by peptide mapping in conjunction with mass spectroscopy or MALDI/TOF/MS), deamidation (evaluated by methods such as ion-exchange chromatography, capillary isoelectric focusing, peptide mapping, isoaspartic acid measurement), and isomerization (evaluated by measuring the isoaspartic acid content, peptide mapping, etc.).

Additionally, or alternatively, the biological activity of the peptide is assayed, for example, by receptor binding assays, competition studies, biomarker studies, or studies of physiological responses typically observed when the peptide is administered in an animal, or by any other assay used to assess a bioactive peptide of interest for activity. In one aspect, the peptide retains at least about 80%, at least about 90%, at least about 95%, or more of the its biological activity after at least about 1 month, at least about 2 months, at least about 3 months, at least about 4 months, at least about 5 months, at least about 6 months, and at least about a year. In certain aspects, the stable lyophilized composition retains biological activity and/or therapeutic activity for at least about 1 month, at least about 2 months, at least about 3 months, at least about 4 months, at least about 5 months, at least about 6 months, and at least about a year at temperatures of at least about 20° C.

The pre-lyophilization formulation can be formulated or provided for lyophilization in a variety of containers. In one aspect, a suitable container permits thermal conductivity, is capable of being tightly sealed at the end of the lyophilization cycle, and/or minimizes the amount of moisture that can permeate its walls and seal. In one aspect, the container is made of a material that offers good thermal conductivity and provides good thermal contact with the source of heat during lyophilization (e.g., the lyophilizer shelf). Suitable materials include, but are not limited to, plastics, glass, and combinations thereof. Other suitable containers are described in U.S. Pat. No. 4,878,597 and are known in the art.

In certain aspects, the internal surface of the material is coated to prevent sticking of the lyophilized composition obtained by lyophilizing the pre-lyophilization formulation. For example, a glass material can be coated with silicone.

In one embodiment, the container is one which is adapted for use in a delivery system for delivering a therapeutically effective amount of the peptide to a subject. For example, the container, may be adapted for attachment to a spray cap for providing a formulation intranasally or can be adapted to complement a pump apparatus, a syringe, or as an injectable cartridge for a pen device. In one aspect, the container has a removable cover or seal which prevents moisture from entering the container during storage of the lyophilized composition and the container, upon removal of the cover or seal, can then be adapted for use in the delivery system. In another aspect, the seal or cover forms an aseptic barrier across the opening of the container. Covers and seals for lyophilization containers are known in the art.

In certain aspects, the cover or seal can be punctured by a component of the delivery system (e.g., a portion of a spray cap), which provides components (e.g., such as water) for reconstituting the lyophilized composition and maintains the reconstituted formulation in a sterile environment.

The container can comprise one or more doses of the formulation. In certain aspects, the container comprises unit doses of the formulation, suitable for administration over a week, two weeks, or a month, where the formulation is administered one time, two times or three times daily.

In certain aspects, the container has a fill-line on its external surface marking the appropriate level to fill the container in order to reconstitute the lyophilized composition obtained after lyophilizing the pre-lyophilization formulation to thereby obtain a ready-to-administer or ready-to-treat formulation.

In one embodiment, the fill volume of the pre-lyophilization formulation is at least about 40% of the final volume. For example, in one aspect, the fill volume of the pre-lyophilization formulation is 4 ml, and the volume of the reconstituted formulation is 10 ml. In certain aspects, the ratio of the fill height to the internal diameter of the container is greater than about 0.5, greater than about 0.75, or is about 1.0. In one aspect, the ratio of the fill height of the pre-lyophilization formulation to the total container height is at least about 25%. In another aspect, the ratio of the fill-height of the pre-lyophilization formulation to the fill height of reconstituted formulation (e.g., in one aspect, formed by adding water to the lyophilized composition obtained after lyophilization of the pre-lyophilization formulation), is at least about 40%, or at least about 45%. In a further aspect, the lyophilized constitution is reconstituted in a ready-to-administer form, by reconstituting in a volume of solution (e.g., water or buffer) which is larger than the volume of the pre-lyophilization formulation. In still another aspect, the ready-to-treat form is in the form of a powder, which can be provided to a patient, e.g., in a spray or aerosol delivery system. Therefore, in certain aspects, the composition may be provided to a subject without reconstituting.

Reconstituted formulations are suitable for administration by a variety of methods, e.g., for transmucosal delivery or for parenteral (e.g., intravenous, intramuscular, intraperitoneal or subcutaneous injection). In one embodiment, the reconstituted formulation is provided intranasally. The formulation can also be provided by eye drop, nasal drop, gargle, inhalation, by topical administration, by spray, or by other methods, such as instillation, metered dose delivery, nebulization, aerosolization, or instillation as suspension in compatible vehicles. Occular, nasal, pulmonary, buccal, sublingual, rectal, or vaginal administration are also contemplated as within the scope of the invention.

In one embodiment, the invention also provides methods for producing lyophilized compositions. Lyophilization systems can be used which are known in the art. Typically, these comprise a drying chamber, one or more, condenser, cooling system, and a mechanism for reducing pressure (e.g., a vacuum chamber).

In one aspect, the components of the pre-lyophilization formulation are combined in a container, such as described above.

In one aspect, the lyophilzation method comprises a freezing stage, a primary drying stage and a secondary drying stage.

Freezing can occur in a single step, by lowering the temperature from a starting temperature to a freezing temperature. In one aspect, the starting temperature ranges from about 0° C.-25° C., or is above 0° C., e.g., from about 10° C. to 25° C. The freezing temperature is selected to optimize crystal formation in the pre-lyophilization formulation. Freezing too rapidly may induce formation of small crystals that can result in higher water vapor resistance and an extended drying time. In one aspect, the difference between the starting temperature and the freezing temperature is at least about 30°, at least about 40°, at least about 50°, or at least about 60°, or at least about 80°. For example, in one aspect, the freezing temperature is from about −40° C. to about −80° C., e.g., the freezing temperature can be about −60° C. Temperature can be lowered gradually, at a constant rate. In one aspect, temperature is lowered about 1° C. per minute.

In certain aspects, the freezing process occurs in a single step. In one aspect, the freezing process excludes an annealing step (e.g., holding at a temperature above the crystallization temperature of a formulation component but below 0° C.). The freezing stage is followed by a primary drying stage. In certain aspects, the primary drying stage follows a period of time at the freezing temperature, for example, the frozen composition can be held at the freezing temperature from 0 minutes to 10 hours, or from about 0 minutes to about 4 hours.

During the primary drying stage, the frozen formulation is subjected to a lower pressure, e.g., by placing the frozen formulation under vacuum. In one aspect, pressure is reduced to 600 mTorr. Further, the frozen formulation is gradually heated (e.g., over 1° C./minute) to cause frozen water to sublime. In one aspect, the formulation is heated to a temperature which is lower than the T_{g} of the component which contributes the majority of the solids content of the pre-lyophilization formulation. For example, in one aspect, the formulation is heated to a temperature which is lower than the component which comprises the hydrophilic portion and hydrophobic or lipophilic portion (e.g., a cyclic oligosaccharide, such as cyclodextrin). In one aspect, the primary drying temperature is lower than −13° C., for example, from about −20° C. to about −16° C. In one aspect, pressure is reduced before the primary drying temperature is reached. In certain aspects, the primary drying temperature is maintained for a time interval, e.g., from about 0 minutes to about 1 hour, or about 30 minutes.

Temperature is again increased gradually (e.g., at 1° C./minute) to desorb remaining bound water during a secondary drying stage. In certain aspects, secondary drying is performed until there is less than about 5% residual water, less than about 2%, or less than about 1% residual water. In one aspect, the secondary drying temperature is a temperature above the final storage temperature for the lyophilized composition, e.g., above 0° C., above 3° C., above 5° C., above 15° C., above 20° C., above 21° C., above 22° C., above 23° C., above 24° C. (e.g., about 25° C.), above 40° C., for example, about 45° C. In one aspect, vacuum is maintained during this process.

In certain aspects, secondary drying occurs in two phases, e.g., temperature is raised to an initial secondary drying temperature, and then is raised again to a final secondary drying temperature. For example, in one aspect, the initial secondary drying temperature is above the T_{g} of the major component of the pre-lyophilization formulation but is below the secondary drying temperature, and in certain instances, is below 0° C., e.g., about −5° C. or about −3° C. The product can be held at the initial secondary drying temperature for a time interval, e.g., from about 0 minutes to about 20 hours, before the product is gradually raised to the final secondary drying temperature, e.g., above 0° C., above 20° C., above 40° C., for example, about 45° C. The product can be held at the final secondary drying temperature for a period of time, e.g., from about 0 minutes to about 10 hours, or about 6 hours. Alternatively, secondary drying can be performed at a single temperature, e.g., gradually raising the product from the primary temperature to a final secondary primary temperature and holding at the final secondary drying temperature for a period of time (e.g., about 5 to about 25 hours, or about 20 hours).

After the secondary drying stage, the product, now a lyophilized composition, in a solid form (e.g., a powder or cake), can be stored for a period of time (e.g., about 0 minutes to about 5 years) at the storage temperature. When ready for use (e.g., administration to a subject), the lyophilized composition can be reconstituted by adding water or buffer and/or additional components as discussed above. In one aspect, the product can be reconstituted to a particle-free solution in less than 30 minutes, e.g., in about 0-15 minutes. In one aspect, the reconstituted product is stable for at least about 48 hours at 0-4° C. In another aspect, the reconstituted product is stable for at least about one week or at least about a month after reconstitution. In still another aspect, the reconstituted product is stable for at least about one week or at least about a month after reconstitution at a temperature greater than 18° C. In certain aspects, the reconstituted product is stable for a period of time which permits a subject to use all the dosage units provided without requiring refrigeration or special storage procedures. For example, if the formulation is provides a one month supply of bioactive peptide, the reconstituted product is stable for at least that period of time (i.e., one month), without refrigeration, e.g., at a temperature greater than 18° C. (e.g., at about 25° C.).

The time of the lyophilization process can vary depending on the fill volume and the solids content, but in one aspect, with a solids content of at least about 20% w/w and a fill volume of the pre-lyophilization solution which is 40% of the final volume of the reconstituted formulation, the process takes under about 48 hours, under about 40 hours, and under about 35 hours.

Peptide formulations as described herein can be used in a variety of methods and generally in any treatment method in which the peptide can be used.

In certain aspects, a peptide formulation can be used to achieve any one or more of a variety of therapeutic effects, including, but not limited to: a glucose-lowering effect, reduction of postprandial glucose, reduction of fasting glucose, reduction in glycemic variability, a glucagon-lowering effect, an insulinotropic effect, modulation of food intake, modulation of appetite, an increase in satiety, an alteration in food preference, a reduction in binge eating, an alteration in weight or rate of change in weight, a decrease in BMI, a reduction in fat without an effect on lean muscle mass, a decrease in fat deposition, modulation of nutrient absorption, improved pancreatic β-cell function, increase in numbers or size of pancreatic β cells, pancreatic β-cell neogenesis, modulation of levels of C-peptide, modulation of apoptosis (e.g., such as a decrease in pancreatic β-cell apoptosis, a reduction in cytokine-mediated apoptosis, a reduction in glucocorticoid-induced apoptosis, a decrease in cardiac myocyte apoptosis, etc.), a modulation of gastric slowing, a modulation of gastric motility, a reduction in markers of oxidative stress, modulation of renal function (e.g., a decrease in glycosuria, etc.), a potentiating interaction with one or more bioactive agents (e.g., such as a bioactive peptide or small molecule), and/or a desired endpoint associated with administration of a peptide, e.g., such as an exendin, amylin, pramlintide, leptin, PYY, calcitonin, or modified, derivative, variant, chimeric and/or hybrid forms thereof.

In certain aspects, a peptide formulation can be used in methods of treatment which include but are not limited to: improving lipid profile (including reducing LDL cholesterol and triglyceride levels and/or changing HDL cholesterol levels), treating hypertension, dyslipidemia, cardiovascular disease, insulin-resistance, treating diabetes mellitus of any kind, including Type I, Type II, and gestational diabetes, diabetes complications (neuropathy), neuropathic pain, retinopathy, nephropathy, conditions of insufficient pancreatic-beta cell mass, treatment of stress hyperglycemia, for treating conditions or disorders associated with toxic hypervolemia, such as renal failure, congestive heart failure, nephrotic syndrome, cirrhosis, pulmonary edema, and hypertension, for treating conditions or disorders that can be alleviated by an increase in cardiac contractility such as congestive heart failure, for treating conditions associated with weight gain (e.g., obesity, for example, having a BMI of 30 or greater), or hunger (e.g., Prader-Willi), loss of body fat (e.g., lipodystrophy), and for treating eating disorders, for treating impaired glucose tolerance (IGT), conditions of impaired fasting plasma glucose, metabolic acidosis, ketosis, arthritis, obesity and osteoporosis.

Methods of treatment will necessarily depend on the bioactive peptide provided in the formulation and should be apparent to those of skill in the art after reading the disclosure herein.

### Example

The following example is provided to illustrate embodiments of the invention and is not intended to be limiting.

### Example 1

A solution was prepared from the following materials and at the following concentrations shown in Table 5 to form a pre-lyophilization formulation of exenatide (synthetic exendin-4).

TABLE 5

| | Pre-lyophilization | | Reconstituted | |
|---|---|---|---|---|
| | formulation (ml) | | Formulation (ml) | |
| | Concentration | Amount | Concentration | Amount |
| Component | (mg/ml) | (mg) | (mg/ml) | (mg) |
| exenatide (synthetic | 7.5 | 30 | 3 | 30 |
| exendin-4) | | | | |
| tartrate buffer | 75 mM | 45 | 30 mM | 45 |
| pH 4.5 | | | (4.5 mg/ml) | |
| methyl-β- | 200 | 800 | 80 | 800 |
| cyclodextrin | | | | |
| DDPC | 5 | 20 | 2 | 20 |
| EDTA | 12.5 | 50 | 5 | 50 |
| BAK | 0.05% | 2 | 0.02% | 2 |
| | (approximately | | (0.2 mg/ml) | |
| | 0.5 mg/ml) | | | |
| Gelatin | 6.25 | 25 | 2.5 | 25 |

% solids: 24.3%

The total height of the vial receiving the pre-lyophilization formulation was 50.62 mm (the thickness of the vial bottom was 2.71 mm). The vial outer diameter (OD) was 23.90 mm while the inner diameter was 19.22 mm. The fill height of the pre-lyophilization formula in the vial was 17.21 to the top of the meniscus, for 4 ml. The fill height of the reconstituted formulation was 34.69 to the top of the meniscus, for 10 ml. Measurements are approximate.

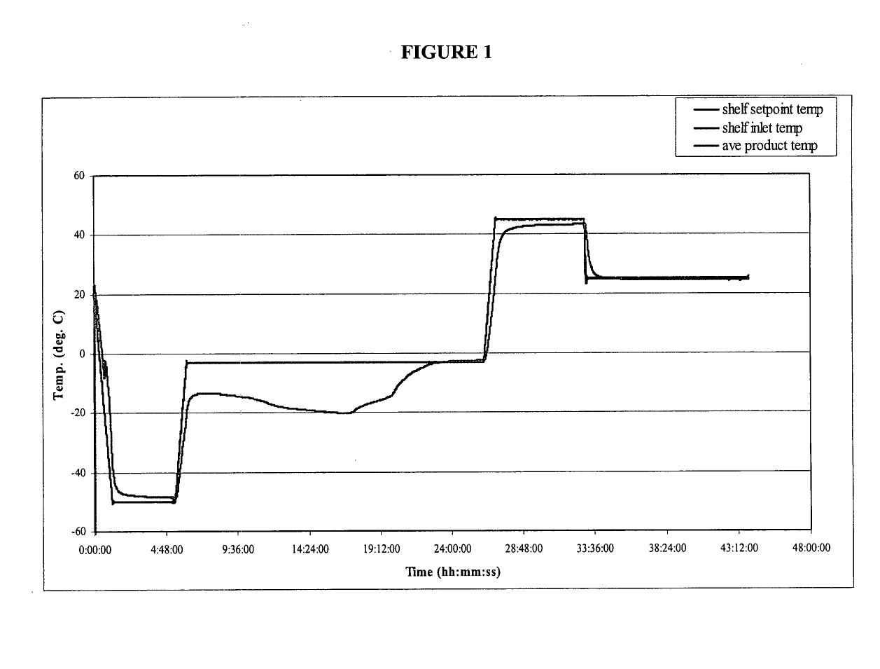

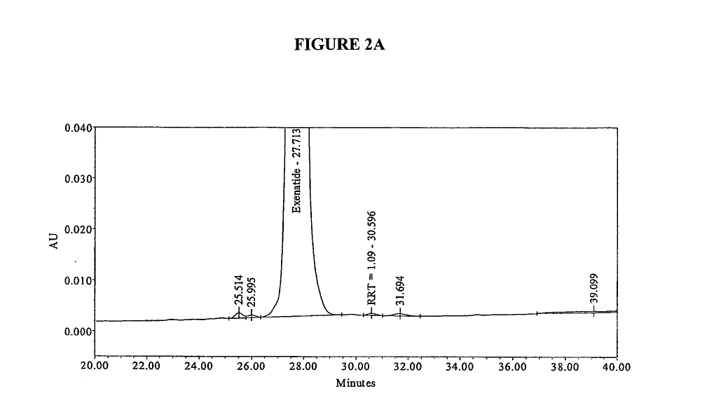

The pre-lyophilization formulation was placed in the lyophilization chamber of a FTS LyoStar II lyophilizer (FTS Systems, Stone Ridge, N.Y.) and subjected to freezing conditions by lowering the temperature from 23° C. to −50° C. at a rate of 1° C./minute. The frozen formulation was maintained at −50° C. for approximately 4 hours. The product temperature was then raised to −16° C. (a shelf temperature of −3° C.) at rate of 1° C./minute for the primary drying stage and the pressure was reduced to 600 mTorr before the formulation was exposed to the primary drying temperature for approximately 20 hours, during which time the product temperature rose from above the Tg of cyclodextrin to the primary drying temperature. The formulation was exposed to a secondary drying stage by raising the temperature to 45° C. at a rate of 1° C./minute and maintaining this temperature for approximately 6 hours. After this time, the temperature was lowered to the storage temperature for the lyophilized composition, i.e., to 25° C. An exemplary lyophilization cycle trace is shown in FIG. 1. The product reconstituted to clarity in 15-25 minutes. The average moisture content for the completely lyophilized product ranged from 2.25% H20 to 0.90% as measured by Karl-Fischer titration (See, e.g., Scholz E. et al, Karl Fischer Titration, Springer Verlag, Berlin, Heidelberg, N.Y., York, Tokyo, 1983).

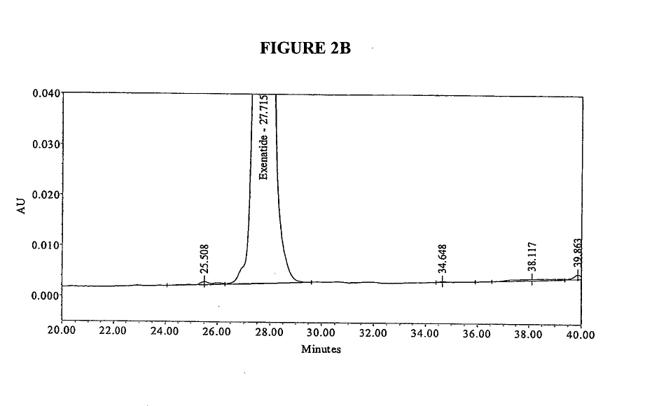

As shown in FIG. 2, 100% of the theoretical content of exenatide can be recovered by this process as determined by SCX-HPLC and comparing the absorbance of the reconstituted formulation at 214 nm as measured by an HPLC photodiode array (PDA) detector and the absorbance of an exenatide-containing standard.

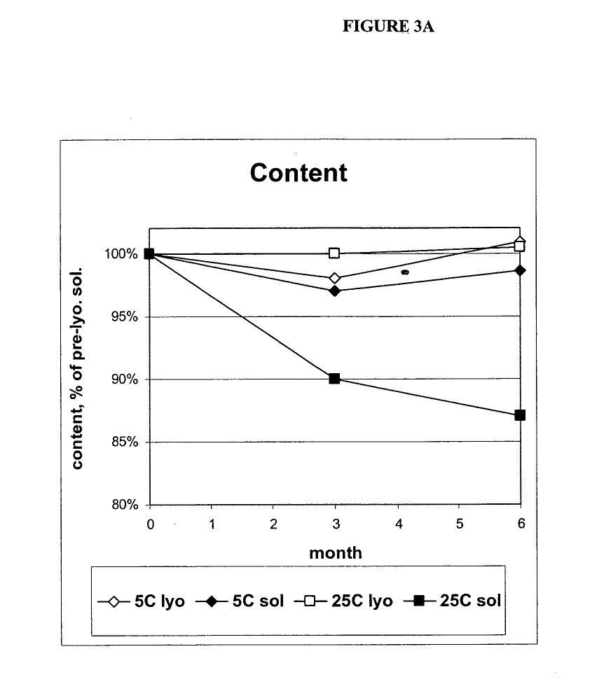

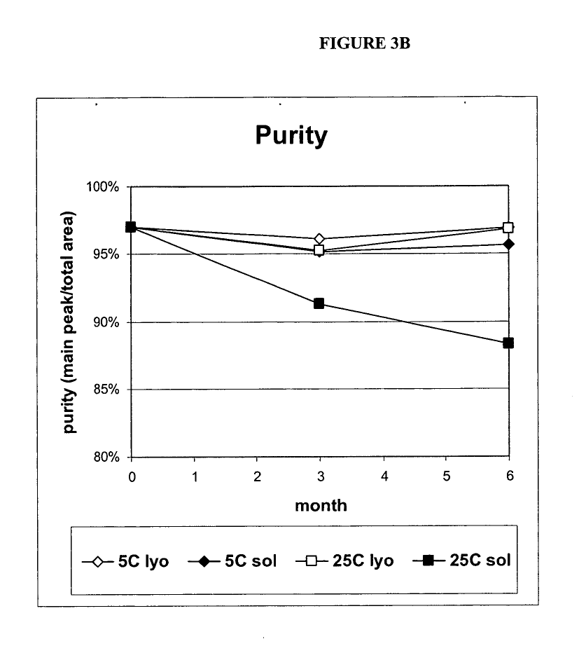

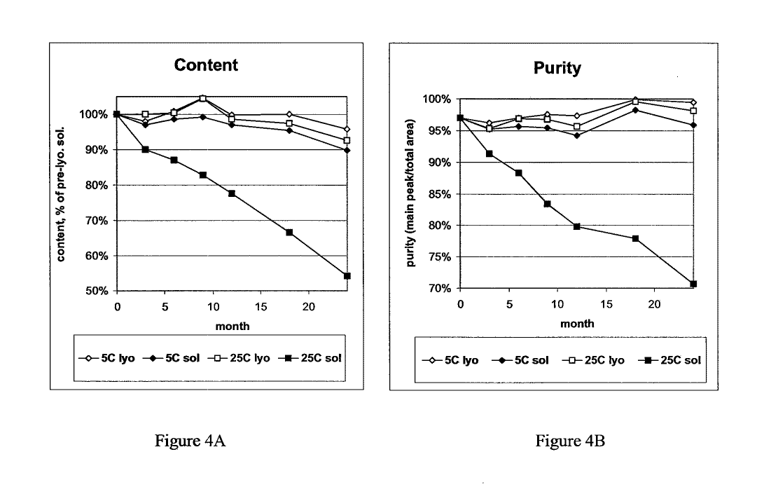

The content and purity of the lyophilized formulation was measured over time for up to six months of storage at both 5° C. and 20° C. as shown in FIGS. 3A and 3B. At each time point, the lyophilized solid was reconstituted with water and the content of exenatide determined by SCX HPLC. “Content” refers to the amount of pure exenatide present in the sample, comparing the exenatide peak area in a lyophilized formulation to peak areas of exenatide standards as well as to exenatide content in the pre-lyophilization formulation. In the figures, % exenatide content is compared-to the exenatide content in the pre-lyophilization formulation which is designated as “100%” . . . Then, for additional comparison, lyophilized samples were reconstituted (t=0) and kept as solutions at 5° C., 25° C., and 40° C. as assayed for content and purity at various time periods and the stability of exenatide in solution (“sol”) was compared to the stability of exenatide in the lyophilized formulation (“lyo”). For example, “5C lyo” is a lyophilized formulation of exenatide stored at 5° C. while “5C sol” refers to the reconstituted formulation of exenatide stored at 5° C. for an identical time period.

As can be seen from the Figures, lyophilized formulations retained over 95% purity over 6 months of storage compared to solution formulations of exenatide which had not been lyophilized and were stored at 5° C. In all cases, the lyophilized formulations were more stable than the solution formulations. Further, the lyophilized formulations showed good stability at 25° C.

### Conclusion

In light of the detailed description of the invention and the example presented above, it can be appreciated that the several aspects of the invention are achieved.

It is to be understood that the present invention has been described in detail by way of illustration and example in order to acquaint others skilled in the art with the invention, its principles, and its practical application. Particular formulations and processes of the present invention are not limited to the descriptions of the specific embodiments presented, but rather the descriptions and examples should be viewed in terms of the claims that follow and their equivalents. While some of the examples and descriptions above include some conclusions about the way the invention may function, the inventors do not intend to be bound by those conclusions and functions, but put them forth only as possible explanations.

It is to be further understood that the specific embodiments of the present invention as set forth are not intended as being exhaustive or limiting of the invention, and that many alternatives, modifications, and variations will be apparent to those of ordinary skill in the art in light of the foregoing examples and detailed description. Accordingly, this invention is intended to embrace all such alternatives, modifications, and variations that fall within the spirit and scope of the following claims.

1-34. (canceled)

35. A pre-lyophilization solution comprising:
a peptide; and
a cyclic oligosaccharide;
wherein the solution comprises a solids content of at least 20% w/w, and wherein the cyclic oligosaccharide provides for at least 80% of the solids content.

36. The solution of claim 35 wherein, the peptide is a bioactive peptide.

37. The solution of claim 35 wherein the peptide is a glucoregulatory peptide.

38. The solution of claim 35 wherein the peptide is a weight-controlling and/or diet-controlling peptide.

39. The solution of claim 35 wherein the peptide is selected from the group consisting of an incretin, an incretin agonist, an amylin, an amylin agonist, calcitonin, a calcitonin agonist, a leptin, a leptin agonist, a PYY antagonist, ghrelin antagonist, and analogs, thereof.

40. The solution of claim 39 wherein the incretin is an exendin, an exendin analog, GLP-1, GLP-1 analog, GIP or a GIP analog.

41. The solution of claim 35 wherein the peptide is fused or conjugated to another bioactive peptide.

42. The solution of claim 35 wherein the solution comprises a preservative which is not benzalkonium chloride and the peptide is an exendin or exendin analog.

43. The solution of claim 42 wherein the preservative comprises paraben.

44. The solution of claim 35 further comprising a polyamino acid.

45. The solution of claim 35 wherein the cyclic oligosaccharide is a cyclodextrin.

46. The solution of claim 35 wherein the solution does not comprise a cryoprotectant or lyoprotectant.

47. A method for preparing a lyophilized peptide composition, comprising:
providing a container comprising a pre-lyophilization solution comprising the peptide, wherein the pre-lyophilization solution comprises a solids content of at least 20% w/w;
lyophilizing the pre-lyophilization solution comprising the peptide, thereby providing a lyophilized peptide composition;
wherein the ratio of the fill height of the pre-lyophilization solution to the container internal diameter is greater than 0.5.

48. The method of claim 47 wherein the ratio of the fill height of the pre-lyophilization solution to the container internal diameter is 0.75 or greater.

49. The method of claim 47 wherein lyophilizing comprises a freezing cycle, a primary drying cycle, and a secondary drying cycle.

50. The method of claim 47 wherein lyophilizing does not include an annealing step.

51. The method of claim 49 wherein the primary drying cycle occurs at a temperature which is below the glass transition temperature of an ingredient in the solution which provides the largest contribution to the solids content.

52. The method of claim 51 wherein the ingredient in the solution which provides the largest contribution to the solids content comprises a cyclic oligosaccharide.

53. The method of claim 49 wherein the secondary drying cycle occurs at greater than 25° C.

54. The method of claim 47 wherein the peptide is a bioactive peptide, and wherein the lyophilized peptide composition is stored at greater than 4° C. and retains biological activity for a period longer than six months.

55. The method of claim 47 wherein lyophilization occurs in under 48 hours.

56. The method of claim 47 further comprising adding a final volume of aqueous solution to the lyophilized peptide composition, wherein the fill volume of the pre-lyophilization solution is 40% of the final volume.

57. A method for preparing a lyophilized peptide composition, comprising:
providing a pre-lyophilization solution comprising the peptide; wherein the pre-lyophilization solution comprises a solids content of at least 20% w/w and further comprises a cyclic oligosaccharide which provides at least 80% of the solids content and lyophilizing the pre-lyophilization solution, thereby obtaining the lyophilized peptide composition.

58. A method for storing a bioactive peptide, comprising preparing a lyophilized peptide composition according to the method of claim 47 and storing the lyophilized peptide composition for at least 48 hours.

59. The method of claim 58 wherein the lyophilized peptide composition is stored for at least one month.

60. The method of claim 58 wherein the lyophilized peptide composition is stored for at least six months.

61. The method of claim 59 wherein the lyophilized peptide composition is stored at a temperature above 18° C.

62. A method of treating a patient treatable with a bioactive peptide comprising administering the peptide in a peptide formulation prepared according to the method of claim 47.


SEQUENCE LISTING (299 sequences)

SEQ ID NO: 1 - protein, 39 aa, Artificial Sequence
  Description of Artificial Sequence Synthetic polypeptide
HSDGTFTSDLSKQMEEEAVRLFIEWLKNGGPSSGAPPPS

SEQ ID NO: 2 - protein, 39 aa, Artificial Sequence
  Description of Artificial Sequence Synthetic polypeptide
HGEGTFTSDLSKQMEEEAVRLFIEWLKNGGPSSGAPPPS

SEQ ID NO: 3 - protein, 39 aa, Artificial Sequence
  Description of Artificial Sequence Synthetic polypeptide
XXXGTXXXXXSKQXEEEAVRLXXXXLKNGGXSSGAXXXX

SEQ ID NO: 4 - protein, 38 aa, Artificial Sequence
  Description of Artificial Sequence Synthetic polypeptide
XXXGXXXXXXXXXXXXXAXXXXXXXXXXGGXSSGAXXX

SEQ ID NO: 5 - protein, 39 aa, Artificial Sequence
  Description of Artificial Sequence Synthetic polypeptide
XXXXXXXXXXXXXXXXXAXXXXXXXXXXGGXSSGAXXXX

SEQ ID NO: 6 - protein, 30 aa, Artificial Sequence
  Description of Artificial Sequence Synthetic polypeptide
HGEGTFTSDLSKQMEEEAVRLFIEWLKNGG

SEQ ID NO: 7 - protein, 30 aa, Artificial Sequence; identical to SEQ ID NO: 6
  Description of Artificial Sequence Synthetic polypeptide

SEQ ID NO: 8 - protein, 28 aa, Artificial Sequence
  Description of Artificial Sequence Synthetic polypeptide
HGEGTFTSDLSKQLEEEAVRLAIEFLKN

SEQ ID NO: 9 - protein, 39 aa, Artificial Sequence
  Description of Artificial Sequence Synthetic polypeptide
HGEGTFTSDLSKQLEEEAVRLFIEFLKNGGPSSGAPPPS

SEQ ID NO: 10 - protein, 39 aa, Artificial Sequence
  Description of Artificial Sequence Synthetic polypeptide
HGEGTFTSDLSKQLEEEAVRLFIEWLKNGGPSSGAPPPS

SEQ ID NO: 11 - protein, 39 aa, Artificial Sequence
  Description of Artificial Sequence Synthetic polypeptide
HGEGTFTSDLSKQMEEEAVRLFIEFLKNGGPSSGAPPPS

SEQ ID NO: 12 - protein, 39 aa, Artificial Sequence
  Description of Artificial Sequence Synthetic polypeptide
YGEGTFTSDLSKQMEEEAVRLFIEWLKNGGPSSGAPPPS

SEQ ID NO: 13 - protein, 39 aa, Artificial Sequence
  Description of Artificial Sequence Synthetic polypeptide
HGEGTFTSDLSKQMEEEAVRLFIEWLKNGGPSSGAPPPY

SEQ ID NO: 14 - protein, 39 aa, Artificial Sequence
  Description of Artificial Sequence Synthetic polypeptide
HGDGTFTSDLSKQMEEEAVRLFIEWLKNGGPSSGAPPPS

SEQ ID NO: 15 - protein, 39 aa, Artificial Sequence
  Description of Artificial Sequence Synthetic polypeptide
HGEGTXTSDLSKQMEEEAVRLFIEWLKNGGPSSGAPPPS

SEQ ID NO: 16 - protein, 39 aa, Artificial Sequence
  Description of Artificial Sequence Synthetic polypeptide
HGEGTFSSDLSKQMEEEAVRLFIEWLKNGGPSSGAPPPS

SEQ ID NO: 17 - protein, 39 aa, Artificial Sequence
  Description of Artificial Sequence Synthetic polypeptide
HGEGTFSTDLSKQMEEEAVRLFIEWLKNGGPSSGAPPPS

SEQ ID NO: 18 - protein, 39 aa, Artificial Sequence
  Description of Artificial Sequence Synthetic polypeptide
HGEGTFTTDLSKQMEEEAVRLFIEWLKNGGPSSGAPPPS

SEQ ID NO: 19 - protein, 39 aa, Artificial Sequence
  Description of Artificial Sequence Synthetic polypeptide
HGEGTFTSELSKQMEEEAVRLFIEWLKNGGPSSGAPPPS

SEQ ID NO: 20 - protein, 39 aa, Artificial Sequence
  Description of Artificial Sequence Synthetic polypeptide
HGEGTFTSDXSKQMEEEAVRLFIEWLKNGGPSSGAPPPS

SEQ ID NO: 21 - protein, 39 aa, Artificial Sequence
  Description of Artificial Sequence Synthetic polypeptide
HGEGTFTSDXSKQLEEEAVRLFIEFLKNGGPSSGAPPPS

SEQ ID NO: 22 - protein, 39 aa, Artificial Sequence
  Description of Artificial Sequence Synthetic polypeptide
HGEGTFTSDLSKQXEEEAVRLFIEWLKNGGPSSGAPPPS

SEQ ID NO: 23 - protein, 39 aa, Artificial Sequence
  Description of Artificial Sequence Synthetic polypeptide
HGEGTFTSDLSKQXEEEAVRLFIEFLKNGGPSSGAPPPS

SEQ ID NO: 24 - protein, 39 aa, Artificial Sequence
  Description of Artificial Sequence Synthetic polypeptide
HGEGTFTSDLSKQMEEEAVRLXIEWLKNGGPSSGAPPPS

SEQ ID NO: 25 - protein, 39 aa, Artificial Sequence
  Description of Artificial Sequence Synthetic polypeptide
HGEGTFTSDLSKQMEEEAVRLFVEWLKNGGPSSGAPPPS

SEQ ID NO: 26 - protein, 39 aa, Artificial Sequence
  Description of Artificial Sequence Synthetic polypeptide
HGEGTFTSDLSKQLEEEAVRLFVEFLKNGGPSSGAPPPS

SEQ ID NO: 27 - protein, 39 aa, Artificial Sequence
  Description of Artificial Sequence Synthetic polypeptide
HGEGTFTSDLSKQMEEEAVRLFXEWLKNGGPSSGAPPPS

SEQ ID NO: 28 - protein, 39 aa, Artificial Sequence
  Description of Artificial Sequence Synthetic polypeptide
HGEGTFTSDLSKQLEEEAVRLFXEFLKNGGPSSGAPPPS

SEQ ID NO: 29 - protein, 39 aa, Artificial Sequence
  Description of Artificial Sequence Synthetic polypeptide
HGEGTFTSDLSKQMEEEAVRLFIDWLKNGGPSSGAPPPS

SEQ ID NO: 30 - protein, 39 aa, Artificial Sequence
  Description of Artificial Sequence Synthetic polypeptide
HAEGTFTSDLSKQLEEEAVRLFIEFLKNGGPSSGAPPPS

SEQ ID NO: 31 - protein, 39 aa, Artificial Sequence
  Description of Artificial Sequence Synthetic polypeptide
HGEGTFTSDLSKQMEEEAVRLFIEWLKNGGXSSGAXXXS

SEQ ID NO: 32 - protein, 39 aa, Artificial Sequence
  Description of Artificial Sequence Synthetic polypeptide
HGEGTFTSDLSKQMEEEAVRLFIEWLKNGGPSSGAXXXS

SEQ ID NO: 33 - protein, 39 aa, Artificial Sequence; identical to SEQ ID NO: 31
  Description of Artificial Sequence Synthetic polypeptide

SEQ ID NO: 34 - protein, 39 aa, Artificial Sequence; identical to SEQ ID NO: 32
  Description of Artificial Sequence Synthetic polypeptide

SEQ ID NO: 35 - protein, 39 aa, Artificial Sequence
  Description of Artificial Sequence Synthetic polypeptide
HGEGTFTSDLSKQLEEEAVRLFIEFLKNGGXSSGAXXXS

SEQ ID NO: 36 - protein, 39 aa, Artificial Sequence; identical to SEQ ID NO: 35
  Description of Artificial Sequence Synthetic polypeptide

SEQ ID NO: 37 - protein, 39 aa, Artificial Sequence; identical to SEQ ID NO: 31
  Description of Artificial Sequence Synthetic polypeptide

SEQ ID NO: 38 - protein, 39 aa, Artificial Sequence; identical to SEQ ID NO: 32
  Description of Artificial Sequence Synthetic polypeptide

SEQ ID NO: 39 - protein, 39 aa, Artificial Sequence; identical to SEQ ID NO: 35
  Description of Artificial Sequence Synthetic polypeptide

SEQ ID NO: 40 - protein, 28 aa, Artificial Sequence
  Description of Artificial Sequence Synthetic polypeptide
HGEGTFTSDLSKQMEEEAVRLFIEWLKN

SEQ ID NO: 41 - protein, 28 aa, Artificial Sequence
  Description of Artificial Sequence Synthetic polypeptide
HGEGTFTSDLSKQLEEEAVRLFIEFLKN

SEQ ID NO: 42 - protein, 28 aa, Artificial Sequence
  Description of Artificial Sequence Synthetic polypeptide
HAEGTFTSDLSKQLEEEAVRLFIEFLKN

SEQ ID NO: 43 - protein, 28 aa, Artificial Sequence
  Description of Artificial Sequence Synthetic polypeptide
HGEGAFTSDLSKQLEEEAVRLFIEFLKN

SEQ ID NO: 44 - protein, 28 aa, Artificial Sequence
  Description of Artificial Sequence Synthetic polypeptide
HGEGTATSDLSKQLEEEAVRLFIEFLKN

SEQ ID NO: 45 - protein, 28 aa, Artificial Sequence
  Description of Artificial Sequence Synthetic polypeptide
HGEGTFTADLSKQLEEEAVRLFIEFLKN

SEQ ID NO: 46 - protein, 28 aa, Artificial Sequence
  Description of Artificial Sequence Synthetic polypeptide
HGEGTFTSDASKQLEEEAVRLFIEFLKN

SEQ ID NO: 47 - protein, 28 aa, Artificial Sequence
  Description of Artificial Sequence Synthetic polypeptide
HGEGTFTSDLAKQLEEEAVRLFIEFLKN

SEQ ID NO: 48 - protein, 28 aa, Artificial Sequence
  Description of Artificial Sequence Synthetic polypeptide
HGEGTFTSDLSAQLEEEAVRLFIEFLKN

SEQ ID NO: 49 - protein, 28 aa, Artificial Sequence
  Description of Artificial Sequence Synthetic polypeptide
HGEGTFTSDLSKALEEEAVRLFIEFLKN

SEQ ID NO: 50 - protein, 28 aa, Artificial Sequence
  Description of Artificial Sequence Synthetic polypeptide
HGEGTFTSDLSKQAEEEAVRLFIEFLKN

SEQ ID NO: 51 - protein, 28 aa, Artificial Sequence
  Description of Artificial Sequence Synthetic polypeptide
HGEGTFTSDLSKQLAEEAVRLFIEFLKN

SEQ ID NO: 52 - protein, 28 aa, Artificial Sequence
  Description of Artificial Sequence Synthetic polypeptide
HGEGTFTSDLSKQLEAEAVRLFIEFLKN

SEQ ID NO: 53 - protein, 28 aa, Artificial Sequence
  Description of Artificial Sequence Synthetic polypeptide
HGEGTFTSDLSKQLEEAAVRLFIEFLKN

SEQ ID NO: 54 - protein, 28 aa, Artificial Sequence
  Description of Artificial Sequence Synthetic polypeptide
HGEGTFTSDLSKQLEEEAARLFIEFLKN

SEQ ID NO: 55 - protein, 28 aa, Artificial Sequence
  Description of Artificial Sequence Synthetic polypeptide
HGEGTFTSDLSKQLEEEAVALFIEFLKN

SEQ ID NO: 56 - protein, 28 aa, Artificial Sequence
  Description of Artificial Sequence Synthetic polypeptide
HGEGTFTSDLSKQLEEEAVRAFIEFLKN

SEQ ID NO: 57 - protein, 28 aa, Artificial Sequence
  Description of Artificial Sequence Synthetic polypeptide
HGEGTFTSDLSKQLEEEAVRLFIAFLKN

SEQ ID NO: 58 - protein, 28 aa, Artificial Sequence
  Description of Artificial Sequence Synthetic polypeptide
HGEGTFTSDLSKQLEEEAVRLFIEALKN

SEQ ID NO: 59 - protein, 28 aa, Artificial Sequence
  Description of Artificial Sequence Synthetic polypeptide
HGEGTFTSDLSKQLEEEAVRLFIEFAKN

SEQ ID NO: 60 - protein, 28 aa, Artificial Sequence
  Description of Artificial Sequence Synthetic polypeptide
HGEGTFTSDLSKQLEEEAVRLFIEFLAN

SEQ ID NO: 61 - protein, 28 aa, Artificial Sequence
  Description of Artificial Sequence Synthetic polypeptide
HGEGTFTSDLSKQLEEEAVRLFIEFLKA

SEQ ID NO: 62 - protein, 38 aa, Artificial Sequence
  Description of Artificial Sequence Synthetic polypeptide
HGEGTFTSDLSKQMEEEAVRLFIEWLKNGGPSSGAPPP

SEQ ID NO: 63 - protein, 38 aa, Artificial Sequence
  Description of Artificial Sequence Synthetic polypeptide
HGEGTFTSDLSKQLEEEAVRLFIEFLKNGGPSSGAPPP

SEQ ID NO: 64 - protein, 37 aa, Artificial Sequence
  Description of Artificial Sequence Synthetic polypeptide
HGEGTFTSDLSKQMEEEAVRLFIEWLKNGGPSSGAPP

SEQ ID NO: 65 - protein, 37 aa, Artificial Sequence
  Description of Artificial Sequence Synthetic polypeptide
HGEGTFTSDLSKQLEEEAVRLFIEFLKNGGPSSGAPP

SEQ ID NO: 66 - protein, 36 aa, Artificial Sequence
  Description of Artificial Sequence Synthetic polypeptide
HGEGTFTSDLSKQMEEEAVRLFIEWLKNGGPSSGAP

SEQ ID NO: 67 - protein, 36 aa, Artificial Sequence
  Description of Artificial Sequence Synthetic polypeptide
HGEGTFTSDLSKQLEEEAVRLFIEFLKNGGPSSGAP

SEQ ID NO: 68 - protein, 35 aa, Artificial Sequence
  Description of Artificial Sequence Synthetic polypeptide
HGEGTFTSDLSKQMEEEAVRLFIEWLKNGGPSSGA

SEQ ID NO: 69 - protein, 35 aa, Artificial Sequence
  Description of Artificial Sequence Synthetic polypeptide
HGEGTFTSDLSKQLEEEAVRLFIEFLKNGGPSSGA

SEQ ID NO: 70 - protein, 34 aa, Artificial Sequence
  Description of Artificial Sequence Synthetic polypeptide
HGEGTFTSDLSKQMEEEAVRLFIEWLKNGGPSSG

SEQ ID NO: 71 - protein, 34 aa, Artificial Sequence
  Description of Artificial Sequence Synthetic polypeptide
HGEGTFTSDLSKQLEEEAVRLFIEFLKNGGPSSG

SEQ ID NO: 72 - protein, 33 aa, Artificial Sequence
  Description of Artificial Sequence Synthetic polypeptide
HGEGTFTSDLSKQMEEEAVRLFIEWLKNGGPSS

SEQ ID NO: 73 - protein, 33 aa, Artificial Sequence
  Description of Artificial Sequence Synthetic polypeptide
HGEGTFTSDLSKQLEEEAVRLFIEFLKNGGPSS

SEQ ID NO: 74 - protein, 32 aa, Artificial Sequence
  Description of Artificial Sequence Synthetic polypeptide
HGEGTFTSDLSKQMEEEAVRLFIEWLKNGGPS

SEQ ID NO: 75 - protein, 32 aa, Artificial Sequence
  Description of Artificial Sequence Synthetic polypeptide
HGEGTFTSDLSKQLEEEAVRLFIEFLKNGGPS

SEQ ID NO: 76 - protein, 31 aa, Artificial Sequence
  Description of Artificial Sequence Synthetic polypeptide
HGEGTFTSDLSKQMEEEAVRLFIEWLKNGGP

SEQ ID NO: 77 - protein, 31 aa, Artificial Sequence
  Description of Artificial Sequence Synthetic polypeptide
HGEGTFTSDLSKQLEEEAVRLFIEFLKNGGP

SEQ ID NO: 78 - protein, 30 aa, Artificial Sequence
  Description of Artificial Sequence Synthetic polypeptide
HGEGTFTSDLSKQLEEEAVRLFIEFLKNGG

SEQ ID NO: 79 - protein, 29 aa, Artificial Sequence
  Description of Artificial Sequence Synthetic polypeptide
HGEGTFTSDLSKQMEEEAVRLFIEWLKNG

SEQ ID NO: 80 - protein, 29 aa, Artificial Sequence
  Description of Artificial Sequence Synthetic polypeptide
HGEGTFTSDLSKQLEEEAVRLFIEFLKNG

SEQ ID NO: 81 - protein, 38 aa, Artificial Sequence
  Description of Artificial Sequence Synthetic polypeptide
HGEGTFTSDLSKQMEEEAVRLFIEWLKNGGXSSGAXXX

SEQ ID NO: 82 - protein, 38 aa, Artificial Sequence
  Description of Artificial Sequence Synthetic polypeptide
HGEGTFTSDLSKQMEEEAVRLFIEWLKNGGPSSGAXXX

SEQ ID NO: 83 - protein, 37 aa, Artificial Sequence
  Description of Artificial Sequence Synthetic polypeptide
HGEGTFTSDLSKQMEEEAVRLFIEWLKNGGXSSGAPP

SEQ ID NO: 84 - protein, 37 aa, Artificial Sequence
  Description of Artificial Sequence Synthetic polypeptide
HGEGTFTSDLSKQMEEEAVRLFIEWLKNGGXSSGAXX

SEQ ID NO: 85 - protein, 37 aa, Artificial Sequence; identical to SEQ ID NO: 84
  Description of Artificial Sequence Synthetic polypeptide

SEQ ID NO: 86 - protein, 36 aa, Artificial Sequence
  Description of Artificial Sequence Synthetic polypeptide
HGEGTFTSDLSKQMEEEAVRLFIEWLKNGGXSSGAX

SEQ ID NO: 87 - protein, 35 aa, Artificial Sequence
  Description of Artificial Sequence Synthetic polypeptide
RGEGTFTSDLSKQMEEEAVRLFIEWLKNGGPSSGA

SEQ ID NO: 88 - protein, 30 aa, Artificial Sequence
  Description of Artificial Sequence Synthetic polypeptide
HGDGTFTSDLSKQMEEEAVRLFIEWLKNGG

SEQ ID NO: 89 - protein, 28 aa, Artificial Sequence
  Description of Artificial Sequence Synthetic polypeptide
HGEGTXTSDLSKQLEEEAVRLFIEFLKN

SEQ ID NO: 90 - protein, 28 aa, Artificial Sequence
  Description of Artificial Sequence Synthetic polypeptide
HGEGTFSSDLSKQMEEEAVRLFIEWLKN

SEQ ID NO: 91 - protein, 28 aa, Artificial Sequence
  Description of Artificial Sequence Synthetic polypeptide
HGEGTFSTDLSKQMEEEAVRLFIEWLKN

SEQ ID NO: 92 - protein, 28 aa, Artificial Sequence
  Description of Artificial Sequence Synthetic polypeptide
HGEGTFTSELSKQMAEEAVRLFIEWLKN

SEQ ID NO: 93 - protein, 28 aa, Artificial Sequence
  Description of Artificial Sequence Synthetic polypeptide
HGEGTFTSDXSKQLEEEAVRLFIEFLKN

SEQ ID NO: 94 - protein, 28 aa, Artificial Sequence
  Description of Artificial Sequence Synthetic polypeptide
HGEGTFTSDLSKQLEEEAVRLXIEFLKN

SEQ ID NO: 95 - protein, 28 aa, Artificial Sequence
  Description of Artificial Sequence Synthetic polypeptide
HGEGTFTSDLSKQMEEEAVRLFXEWLKN

SEQ ID NO: 96 - protein, 28 aa, Artificial Sequence
  Description of Artificial Sequence Synthetic polypeptide
HGEGTFTSDLSKQLEEEAVRLFIDFLKN

SEQ ID NO: 97 - protein, 33 aa, Artificial Sequence
  Description of Artificial Sequence Synthetic polypeptide
HGEGTFTSDASKQLEEEAVRLFIEFLKNGGPSS

SEQ ID NO: 98 - protein, 29 aa, Artificial Sequence
  Description of Artificial Sequence Synthetic polypeptide
HGEGTFTSDASKQMEEEAVRLFIEWLKNG

SEQ ID NO: 99 - protein, 37 aa, Artificial Sequence
  Description of Artificial Sequence Synthetic polypeptide
HGEGTFTSDASKQMEEEAVRLFIEWLKNGGXSSGAXX

SEQ ID NO: 100 - protein, 28 aa, Artificial Sequence
  Description of Artificial Sequence Synthetic polypeptide
AGEGTFTSDLSKQLEEEAVRLFIEFLKN

SEQ ID NO: 101 - protein, 28 aa, Artificial Sequence
  Description of Artificial Sequence Synthetic polypeptide
HGAGTFTSDLSKQLEEEAVRLFIEFLKN

SEQ ID NO: 102 - protein, 28 aa, Artificial Sequence
  Description of Artificial Sequence Synthetic polypeptide
HGEATFTSDLSKQLEEEAVRLFIEFLKN

SEQ ID NO: 103 - protein, 28 aa, Artificial Sequence
  Description of Artificial Sequence Synthetic polypeptide
HGEGTFTSALSKQLEEEAVRLFIEFLKN

SEQ ID NO: 104 - protein, 28 aa, Artificial Sequence
  Description of Artificial Sequence Synthetic polypeptide
AGEGTFTSDLSKQMEEEAVRLFIEWLKN

SEQ ID NO: 105 - protein, 28 aa, Artificial Sequence
  Description of Artificial Sequence Synthetic polypeptide
HGAGTFTSDLSKQMEEEAVRLFIEWLKN

SEQ ID NO: 106 - protein, 28 aa, Artificial Sequence
  Description of Artificial Sequence Synthetic polypeptide
HGEATFTSDLSKQMEEEAVRLFIEWLKN

SEQ ID NO: 107 - protein, 28 aa, Artificial Sequence
  Description of Artificial Sequence Synthetic polypeptide
HGEGTFTSALSKQMEEEAVRLFIEWLKN

SEQ ID NO: 108 - protein, 28 aa, Artificial Sequence
  Description of Artificial Sequence Synthetic polypeptide
HGEGTFTSDASKQMEEEAVRLFIEWLKN

SEQ ID NO: 109 - protein, 28 aa, Artificial Sequence
  Description of Artificial Sequence Synthetic polypeptide
AAEGTFTSDLSKQMEEEAVRLFIEWLKN

SEQ ID NO: 110 - protein, 28 aa, Artificial Sequence
  Description of Artificial Sequence Synthetic polypeptide
AAEGTFTSDLSKQLEEEAVRLFIEFLKN

SEQ ID NO: 111 - protein, 28 aa, Artificial Sequence
  Description of Artificial Sequence Synthetic polypeptide
AGDGTFTSDLSKQMEEEAVRLFIEWLKN

SEQ ID NO: 112 - protein, 28 aa, Artificial Sequence
  Description of Artificial Sequence Synthetic polypeptide
AGDGTFTSDLSKQLEEEAVRLFIEFLKN

SEQ ID NO: 113 - protein, 28 aa, Artificial Sequence
  Description of Artificial Sequence Synthetic polypeptide
AGDGAFTSDLSKQMEEEAVRLFIEWLKN

SEQ ID NO: 114 - protein, 28 aa, Artificial Sequence
  Description of Artificial Sequence Synthetic polypeptide
AGDGAFTSDLSKQLEEEAVRLFIEFLKN

SEQ ID NO: 115 - protein, 28 aa, Artificial Sequence
  Description of Artificial Sequence Synthetic polypeptide
AGDGTXTSDLSKQMEEEAVRLFIEWLKN

SEQ ID NO: 116 - protein, 28 aa, Artificial Sequence
  Description of Artificial Sequence Synthetic polypeptide
AGDGTXTSDLSKQLEEEAVRLFIEFLKN

SEQ ID NO: 117 - protein, 28 aa, Artificial Sequence
  Description of Artificial Sequence Synthetic polypeptide
AGDGTFSSDLSKQMEEEAVRLFIEWLKN

SEQ ID NO: 118 - protein, 28 aa, Artificial Sequence
  Description of Artificial Sequence Synthetic polypeptide
AGDGTFSSDLSKQLEEEAVRLFIEFLKN

SEQ ID NO: 119 - protein, 28 aa, Artificial Sequence
  Description of Artificial Sequence Synthetic polypeptide
AGDGTFTADLSKQMEEEAVRLFIEWLKN

SEQ ID NO: 120 - protein, 28 aa, Artificial Sequence
  Description of Artificial Sequence Synthetic polypeptide
AGDGTFTADLSKQLEEEAVRLFIEFLKN

SEQ ID NO: 121 - protein, 28 aa, Artificial Sequence
  Description of Artificial Sequence Synthetic polypeptide
AGDGTFTSALSKQMEEEAVRLFIEWLKN

SEQ ID NO: 122 - protein, 28 aa, Artificial Sequence
  Description of Artificial Sequence Synthetic polypeptide
AGDGTFTSALSKQLEEEAVRLFIEFLKN

SEQ ID NO: 123 - protein, 28 aa, Artificial Sequence
  Description of Artificial Sequence Synthetic polypeptide
AGDGTFTSELSKQMEEEAVRLFIEWLKN

SEQ ID NO: 124 - protein, 28 aa, Artificial Sequence
  Description of Artificial Sequence Synthetic polypeptide
AGDGTFTSELSKQLEEEAVRLFIEFLKN

SEQ ID NO: 125 - protein, 28 aa, Artificial Sequence
  Description of Artificial Sequence Synthetic polypeptide
AGDGTFTSDASKQMEEEAVRLFIEWLKN

SEQ ID NO: 126 - protein, 28 aa, Artificial Sequence
  Description of Artificial Sequence Synthetic polypeptide
AGDGTFTSDASKQLEEEAVRLFIEFLKN

SEQ ID NO: 127 - protein, 28 aa, Artificial Sequence
  Description of Artificial Sequence Synthetic polypeptide
AGDGTFTSDXSKQMEEEAVRLFIEWLKN

SEQ ID NO: 128 - protein, 28 aa, Artificial Sequence
  Description of Artificial Sequence Synthetic polypeptide
AGDGTFTSDXSKQLEEEAVRLFIEFLKN

SEQ ID NO: 129 - protein, 28 aa, Artificial Sequence
  Description of Artificial Sequence Synthetic polypeptide
AGDGTFTSDLAKQMEEEAVRLFIEWLKN

SEQ ID NO: 130 - protein, 28 aa, Artificial Sequence
  Description of Artificial Sequence Synthetic polypeptide
AGDGTFTSDLAKQLEEEAVRLFIEFLKN

SEQ ID NO: 131 - protein, 28 aa, Artificial Sequence
  Description of Artificial Sequence Synthetic polypeptide
AGDGTFTSDLSAQMEEEAVRLFIEWLKN

SEQ ID NO: 132 - protein, 28 aa, Artificial Sequence
  Description of Artificial Sequence Synthetic polypeptide
AGDGTFTSDLSAQLEEEAVRLFIEFLKN

SEQ ID NO: 133 - protein, 28 aa, Artificial Sequence
  Description of Artificial Sequence Synthetic polypeptide
AGDGTFTSDLSKAMEEEAVRLFIEWLKN

SEQ ID NO: 134 - protein, 28 aa, Artificial Sequence
  Description of Artificial Sequence Synthetic polypeptide
AGDGTFTSDLSKALEEEAVRLFIEFLKN

SEQ ID NO: 135 - protein, 28 aa, Artificial Sequence
  Description of Artificial Sequence Synthetic polypeptide
AGDGTFTSDLSKQAEEEAVRLFIEWLKN

SEQ ID NO: 136 - protein, 28 aa, Artificial Sequence
  Description of Artificial Sequence Synthetic polypeptide
AGDGTFTSDLSKQAEEEAVRLFIEFLKN

SEQ ID NO: 137 - protein, 28 aa, Artificial Sequence
  Description of Artificial Sequence Synthetic polypeptide
AGDGTFTSDLSKQXEEEAVRLFIEWLKN

SEQ ID NO: 138 - protein, 28 aa, Artificial Sequence
  Description of Artificial Sequence Synthetic polypeptide
AGDGTFTSDLSKQXEEEAVRLFIEFLKN

SEQ ID NO: 139 - protein, 28 aa, Artificial Sequence
  Description of Artificial Sequence Synthetic polypeptide
AGDGTFTSDLSKQMAEEAVRLFIEWLKN

SEQ ID NO: 140 - protein, 28 aa, Artificial Sequence
  Description of Artificial Sequence Synthetic polypeptide
AGDGTFTSDLSKQLAEEAVRLFIEFLKN

SEQ ID NO: 141 - protein, 28 aa, Artificial Sequence
  Description of Artificial Sequence Synthetic polypeptide
AGDGTFTSDLSKQMEAEAVRLFIEWLKN

SEQ ID NO: 142 - protein, 28 aa, Artificial Sequence
  Description of Artificial Sequence Synthetic polypeptide
AGDGTFTSDLSKQLEAEAVRLFIEFLKN

SEQ ID NO: 143 - protein, 28 aa, Artificial Sequence
  Description of Artificial Sequence Synthetic polypeptide
AGDGTFTSDLSKQMEEAAVRLFIEWLKN

SEQ ID NO: 144 - protein, 28 aa, Artificial Sequence
  Description of Artificial Sequence Synthetic polypeptide
AGDGTFTSDLSKQLEEAAVRLFIEFLKN

SEQ ID NO: 145 - protein, 28 aa, Artificial Sequence
  Description of Artificial Sequence Synthetic polypeptide
AGDGTFTSDLSKQMEEEAARLFIEWLKN

SEQ ID NO: 146 - protein, 28 aa, Artificial Sequence
  Description of Artificial Sequence Synthetic polypeptide
AGDGTFTSDLSKQLEEEAARLFIEFLKN

SEQ ID NO: 147 - protein, 28 aa, Artificial Sequence
  Description of Artificial Sequence Synthetic polypeptide
AGDGTFTSDLSKQMEEEAVALFIEWLKN

SEQ ID NO: 148 - protein, 28 aa, Artificial Sequence
  Description of Artificial Sequence Synthetic polypeptide
AGDGTFTSDLSKQLEEEAVALFIEFLKN

SEQ ID NO: 149 - protein, 28 aa, Artificial Sequence
  Description of Artificial Sequence Synthetic polypeptide
AGDGTFTSDLSKQMEEEAVRAFIEWLKN

SEQ ID NO: 150 - protein, 28 aa, Artificial Sequence
  Description of Artificial Sequence Synthetic polypeptide
AGDGTFTSDLSKQLEEEAVRAFIEFLKN

SEQ ID NO: 151 - protein, 28 aa, Artificial Sequence
  Description of Artificial Sequence Synthetic polypeptide
AGDGTFTSDLSKQMEEEAVRLXIEWLKN

SEQ ID NO: 152 - protein, 28 aa, Artificial Sequence
  Description of Artificial Sequence Synthetic polypeptide
AGDGTFTSDLSKQLEEEAVRLXIEFLKN

SEQ ID NO: 153 - protein, 28 aa, Artificial Sequence
  Description of Artificial Sequence Synthetic polypeptide
AGDGTFTSDLSKQMEEEAVRLFVEWLKN

SEQ ID NO: 154 - protein, 28 aa, Artificial Sequence
  Description of Artificial Sequence Synthetic polypeptide
AGDGTFTSDLSKQLEEEAVRLFVEFLKN

SEQ ID NO: 155 - protein, 28 aa, Artificial Sequence
  Description of Artificial Sequence Synthetic polypeptide
AGDGTFTSDLSKQMEEEAVRLFXEWLKN

SEQ ID NO: 156 - protein, 28 aa, Artificial Sequence
  Description of Artificial Sequence Synthetic polypeptide
AGDGTFTSDLSKQLEEEAVRLFXEFLKN

SEQ ID NO: 157 - protein, 28 aa, Artificial Sequence
  Description of Artificial Sequence Synthetic polypeptide
AGDGTFTSDLSKQMEEEAVRLFIDWLKN

SEQ ID NO: 158 - protein, 28 aa, Artificial Sequence
  Description of Artificial Sequence Synthetic polypeptide
AGDGTFTSDLSKQLEEEAVRLFIDFLKN

SEQ ID NO: 159 - protein, 28 aa, Artificial Sequence
  Description of Artificial Sequence Synthetic polypeptide
AGDGTFTSDLSKQMEEEAVRLFIEALKN

SEQ ID NO: 160 - protein, 28 aa, Artificial Sequence
  Description of Artificial Sequence Synthetic polypeptide
AGDGTFTSDLSKQLEEEAVRLFIEALKN

SEQ ID NO: 161 - protein, 28 aa, Artificial Sequence
  Description of Artificial Sequence Synthetic polypeptide
AGDGTFTSDLSKQMEEEAVRLFIEWAKN

SEQ ID NO: 162 - protein, 28 aa, Artificial Sequence
  Description of Artificial Sequence Synthetic polypeptide
AGDGTFTSDLSKQLEEEAVRLFIEFAKN

SEQ ID NO: 163 - protein, 28 aa, Artificial Sequence
  Description of Artificial Sequence Synthetic polypeptide
AGDGTFTSDLSKQMEEEAVRLFIEWLAN

SEQ ID NO: 164 - protein, 28 aa, Artificial Sequence
  Description of Artificial Sequence Synthetic polypeptide
AGDGTFTSDLSKQLEEEAVRLFIEFLAN

SEQ ID NO: 165 - protein, 28 aa, Artificial Sequence
  Description of Artificial Sequence Synthetic polypeptide
AGDGTFTSDLSKQMEEEAVRLFIEWLKA

SEQ ID NO: 166 - protein, 28 aa, Artificial Sequence
  Description of Artificial Sequence Synthetic polypeptide
AGDGTFTSDLSKQLEEEAVRLFIEFLKA

SEQ ID NO: 167 - protein, 38 aa, Artificial Sequence
  Description of Artificial Sequence Synthetic polypeptide
AGEGTFTSDLSKQMEEEAVRLFIEWLKNGGPSSGAPPP

SEQ ID NO: 168 - protein, 38 aa, Artificial Sequence
  Description of Artificial Sequence Synthetic polypeptide
HGAGTFTSDLSKQLEEEAVRLFIEFLKNGGPSSGAPPP

SEQ ID NO: 169 - protein, 37 aa, Artificial Sequence
  Description of Artificial Sequence Synthetic polypeptide
HGEATFTSDLSKQMEEEAVRLFIEWLKNGGPSSGAPP

SEQ ID NO: 170 - protein, 36 aa, Artificial Sequence
  Description of Artificial Sequence Synthetic polypeptide
HGEGTFTSALSKQMEEEAVRLFIEWLKNGGPSSGAP

SEQ ID NO: 171 - protein, 36 aa, Artificial Sequence
  Description of Artificial Sequence Synthetic polypeptide
AGEGTFTSDASKQLEEEAVRLFIEFLKNGGPSSGAP

SEQ ID NO: 172 - protein, 35 aa, Artificial Sequence
  Description of Artificial Sequence Synthetic polypeptide
AGEGTFTSDLSKQMEEEAVRLFIEWLKNGGPSSGA

SEQ ID NO: 173 - protein, 35 aa, Artificial Sequence
  Description of Artificial Sequence Synthetic polypeptide
HGAGTFTSDLSKQLEEEAVRLFIEFLKNGGPSSGA

SEQ ID NO: 174 - protein, 34 aa, Artificial Sequence
  Description of Artificial Sequence Synthetic polypeptide
HGEATFTSDLSKQMEEEAVRLFIEWLKNGGPSSG

SEQ ID NO: 175 - protein, 33 aa, Artificial Sequence
  Description of Artificial Sequence Synthetic polypeptide
HGEGTFTSALSKQMEEEAVRLFIEWLKNGGPSS

SEQ ID NO: 176 - protein, 32 aa, Artificial Sequence
  Description of Artificial Sequence Synthetic polypeptide
AGEGTFTSDLSKQMEEEAVRLFIEWLKNGGPS

SEQ ID NO: 177 - protein, 32 aa, Artificial Sequence
  Description of Artificial Sequence Synthetic polypeptide
HGAGTFTSDLSKQLEEEAVRLFIEFLKNGGPS

SEQ ID NO: 178 - protein, 31 aa, Artificial Sequence
  Description of Artificial Sequence Synthetic polypeptide
HGEATFTSDLSKQMEEEAVRLFIEWLKNGGP

SEQ ID NO: 179 - protein, 30 aa, Artificial Sequence
  Description of Artificial Sequence Synthetic polypeptide
HGEGTFTSALSKQLEEEAVRLFIEFLKNGG

SEQ ID NO: 180 - protein, 29 aa, Artificial Sequence
  Description of Artificial Sequence Synthetic polypeptide
AGEGTFTSDLSKQLEEEAVRLFIEFLKNG

SEQ ID NO: 181 - protein, 38 aa, Artificial Sequence
  Description of Artificial Sequence Synthetic polypeptide
HGAGTFTSDLSKQMEEEAVRLFIEWLKNGGXSSGAXXX

SEQ ID NO: 182 - protein, 38 aa, Artificial Sequence
  Description of Artificial Sequence Synthetic polypeptide
HGEATFTSDLSKQMEEEAVRLFIEWLKNGGPSSGAXXX

SEQ ID NO: 183 - protein, 37 aa, Artificial Sequence
  Description of Artificial Sequence Synthetic polypeptide
HGEGTFTSALSKQMEEEAVRLFIEWLKNGGXSSGAXX

SEQ ID NO: 184 - protein, 36 aa, Artificial Sequence
  Description of Artificial Sequence Synthetic polypeptide
AGEGTFTSDLSKQMEEEAVRLFIEWLKNGGXSSGAX

SEQ ID NO: 185 - protein, 35 aa, Artificial Sequence
  Description of Artificial Sequence Synthetic polypeptide
HGAGTFTSDLSKQMEEEAVRLFIEWLKNGGPSSGA

SEQ ID NO: 186 - protein, 30 aa, Artificial Sequence
  Description of Artificial Sequence Synthetic polypeptide
HGDATFTSDLSKQMEEEAVRLFIEWLKNGG

SEQ ID NO: 187 - protein, 39 aa, Artificial Sequence
  Description of Artificial Sequence Synthetic polypeptide
AGEGTFTSDLSKQMEEEAVRLFIEWLKNGGPSSGAPPPS

SEQ ID NO: 188 - protein, 39 aa, Artificial Sequence
  Description of Artificial Sequence Synthetic polypeptide
AGAGTFTSDLSKQLEEEAVRLFIEFLKNGGPSSGAPPPS

SEQ ID NO: 189 - protein, 36 aa, Artificial Sequence
  Description of Artificial Sequence Synthetic polypeptide
APLEPVYPGDNATPEQMAQYAADLRRYINMLTRPRY

SEQ ID NO: 190 - protein, 36 aa, Artificial Sequence
  Description of Artificial Sequence Synthetic polypeptide
YPIKPEAPGEDASPEELNRYYASLRHYLNLVTRQRY

SEQ ID NO: 191 - protein, 34 aa, Artificial Sequence
  Description of Artificial Sequence Synthetic polypeptide
IKPEAPGEDASPEELNRYYASLRHYLNLVTRQRY

SEQ ID NO: 192 - protein, 36 aa, Artificial Sequence
  Description of Artificial Sequence Synthetic polypeptide
YPSKPDNPGEDAPAEDMARYYSALRHYINLITRQRY

SEQ ID NO: 193 - protein, 34 aa, Artificial Sequence
  Description of Artificial Sequence Synthetic polypeptide
SKPDNPGEDAPAEDMARYYSALRHYINLITRQRY

SEQ ID NO: 194 - protein, 15 aa, Artificial Sequence
  Description of Artificial Sequence Synthetic polypeptide
ASLRHYLNLVTRQRY

SEQ ID NO: 195 - protein, 31 aa, Artificial Sequence
  Description of Artificial Sequence Synthetic polypeptide
HVEGTFTSDVSSYLEEQAAKEFIAWLVKGRG

SEQ ID NO: 196 - protein, 31 aa, Artificial Sequence
  Description of Artificial Sequence Synthetic polypeptide
HVEGTFTSDVSSYLEDQAAKEFIAWLVKGRG

SEQ ID NO: 197 - protein, 31 aa, Artificial Sequence
  Description of Artificial Sequence Synthetic polypeptide
HVEGTFTSDVSSYLERQAAKEFIAWLVKGRG

SEQ ID NO: 198 - protein, 31 aa, Artificial Sequence
  Description of Artificial Sequence Synthetic polypeptide
HVEGTFTSDVSSYLEKQAAKEFIAWLVKGRG

SEQ ID NO: 199 - protein, 31 aa, Artificial Sequence
  Description of Artificial Sequence Synthetic polypeptide
HGEGTFTSDVSSYLEEQAAKEFIAWLVKGRG

SEQ ID NO: 200 - protein, 31 aa, Artificial Sequence
  Description of Artificial Sequence Synthetic polypeptide
HGEGTFTSDVSSYLEDQAAKEFIAWLVKGRG

SEQ ID NO: 201 - protein, 31 aa, Artificial Sequence
  Description of Artificial Sequence Synthetic polypeptide
HGEGTFTSDVSSYLERQAAKEFIAWLVKGRG

SEQ ID NO: 202 - protein, 31 aa, Artificial Sequence
  Description of Artificial Sequence Synthetic polypeptide
HGEGTFTSDVSSYLEKQAAKEFIAWLVKGRG

SEQ ID NO: 203 - protein, 31 aa, Artificial Sequence
  Description of Artificial Sequence Synthetic polypeptide
HVEGTFTSDVSSYLEGQAAKEFIEWLVKGRG

SEQ ID NO: 204 - protein, 31 aa, Artificial Sequence
  Description of Artificial Sequence Synthetic polypeptide
HGEGTFTSDVSSYLEGQAAKEFIEWLVKGRG

SEQ ID NO: 205 - protein, 31 aa, Artificial Sequence
  Description of Artificial Sequence Synthetic polypeptide
HVEGTFTSDVSSYLEGQAAKEFIAWLVKGRH

SEQ ID NO: 206 - protein, 31 aa, Artificial Sequence
  Description of Artificial Sequence Synthetic polypeptide
HGEGTFTSDVSSYLEGQAAKEFIAWLVKGRH

SEQ ID NO: 207 - protein, 31 aa, Artificial Sequence
  Description of Artificial Sequence Synthetic polypeptide
HVEGTFTSDVSSYLEEQAAKAFIAWLVKGRH

SEQ ID NO: 208 - protein, 31 aa, Artificial Sequence
  Description of Artificial Sequence Synthetic polypeptide
HVEGTFTSDVSSYLEKEAAKEFIAWLVKGRH

SEQ ID NO: 209 - protein, 31 aa, Artificial Sequence
  Description of Artificial Sequence Synthetic polypeptide
HAEGTFTSDVSSYLEGQAAKEFIAWLVKGRG

SEQ ID NO: 210 - protein, 31 aa, Artificial Sequence
  Description of Artificial Sequence Synthetic polypeptide
HAEGTFTSDVSSYLEEQAAKEFIAWLVKGRG

SEQ ID NO: 211 - protein, 31 aa, Artificial Sequence
  Description of Artificial Sequence Synthetic polypeptide
HAEGTFTSDVSSYLEDQAAKEFIAWLVKGRG

SEQ ID NO: 212 - protein, 31 aa, Artificial Sequence
  Description of Artificial Sequence Synthetic polypeptide
HAEGTFTSDVSSYLERQAAKEFIAWLVKGRG

SEQ ID NO: 213 - protein, 31 aa, Artificial Sequence
  Description of Artificial Sequence Synthetic polypeptide
HAEGTFTSDVSSYLEKQAAKEFIAWLVKGRG

SEQ ID NO: 214 - protein, 31 aa, Artificial Sequence
  Description of Artificial Sequence Synthetic polypeptide
HAEGTFTSDVSSYLEXQAAKEFIAWLVKGRG

SEQ ID NO: 215 - protein, 31 aa, Artificial Sequence
  Description of Artificial Sequence Synthetic polypeptide
HVEGTFTSDVSSYLEXQAAKEFIAWLVKGRG

SEQ ID NO: 216 - protein, 31 aa, Artificial Sequence
  Description of Artificial Sequence Synthetic polypeptide
HGEGTFTSDVSSYLEXQAAKEFIAWLVKGRG

SEQ ID NO: 217 - protein, 30 aa, Artificial Sequence
  Description of Artificial Sequence Synthetic polypeptide
HAEGTFTSDVSSYLEEQAAKEFIAWLVKGR

SEQ ID NO: 218 - protein, 30 aa, Artificial Sequence
  Description of Artificial Sequence Synthetic polypeptide
HAEGTFTSDVSSYLEDQAAKEFIAWLVKGR

SEQ ID NO: 219 - protein, 30 aa, Artificial Sequence
  Description of Artificial Sequence Synthetic polypeptide
HAEGTFTSDVSSYLERQAAKEFIAWLVKGR

SEQ ID NO: 220 - protein, 30 aa, Artificial Sequence
  Description of Artificial Sequence Synthetic polypeptide
HAEGTFTSDVSSYLEKQAAKEFIAWLVKGR

SEQ ID NO: 221 - protein, 30 aa, Artificial Sequence
  Description of Artificial Sequence Synthetic polypeptide
HAEGTFTSDVSSYLEXQAAKEFIAWLVKGR

SEQ ID NO: 222 - protein, 30 aa, Artificial Sequence
  Description of Artificial Sequence Synthetic polypeptide
HVEGTFTSDVSSYLEEQAAKEFIAWLVKGR

SEQ ID NO: 223 - protein, 30 aa, Artificial Sequence
  Description of Artificial Sequence Synthetic polypeptide
HVEGTFTSDVSSYLEDQAAKEFIAWLVKGR

SEQ ID NO: 224 - protein, 30 aa, Artificial Sequence
  Description of Artificial Sequence Synthetic polypeptide
HVEGTFTSDVSSYLERQAAKEFIAWLVKGR

SEQ ID NO: 225 - protein, 30 aa, Artificial Sequence
  Description of Artificial Sequence Synthetic polypeptide
HVEGTFTSDVSSYLEKQAAKEFIAWLVKGR

SEQ ID NO: 226 - protein, 30 aa, Artificial Sequence
  Description of Artificial Sequence Synthetic polypeptide
HVEGTFTSDVSSYLEXQAAKEFIAWLVKGR

SEQ ID NO: 227 - protein, 30 aa, Artificial Sequence
  Description of Artificial Sequence Synthetic polypeptide
HGEGTFTSDVSSYLEEQAAKEFIAWLVKGR

SEQ ID NO: 228 - protein, 30 aa, Artificial Sequence
  Description of Artificial Sequence Synthetic polypeptide
HGEGTFTSDVSSYLEDQAAKEFIAWLVKGR

SEQ ID NO: 229 - protein, 30 aa, Artificial Sequence
  Description of Artificial Sequence Synthetic polypeptide
HGEGTFTSDVSSYLERQAAKEFIAWLVKGR

SEQ ID NO: 230 - protein, 30 aa, Artificial Sequence
  Description of Artificial Sequence Synthetic polypeptide
HGEGTFTSDVSSYLEKQAAKEFIAWLVKGR

SEQ ID NO: 231 - protein, 30 aa, Artificial Sequence
  Description of Artificial Sequence Synthetic polypeptide
HGEGTFTSDVSSYLEXQAAKEFIAWLVKGR

SEQ ID NO: 232 - protein, 31 aa, Artificial Sequence
  Description of Artificial Sequence Synthetic polypeptide
HVEGTFTSDVSSYLEGKAAKEFIAWLVKGRG

SEQ ID NO: 233 - protein, 31 aa, Artificial Sequence
  Description of Artificial Sequence Synthetic polypeptide
HVEGTFTSDVSSYLEGQAAKAFIAWLVKGRG

SEQ ID NO: 234 - protein, 31 aa, Artificial Sequence; identical to SEQ ID NO: 203
  Description of Artificial Sequence Synthetic polypeptide

SEQ ID NO: 235 - protein, 31 aa, Artificial Sequence; identical to SEQ ID NO: 204
  Description of Artificial Sequence Synthetic polypeptide

SEQ ID NO: 236 - protein, 31 aa, Artificial Sequence
  Description of Artificial Sequence Synthetic polypeptide
HVEGTFTSDVSSYLEGQAAKEFIAWLVKHRG

SEQ ID NO: 237 - protein, 31 aa, Artificial Sequence; identical to SEQ ID NO: 205
  Description of Artificial Sequence Synthetic polypeptide

SEQ ID NO: 238 - protein, 31 aa, Artificial Sequence
  Description of Artificial Sequence Synthetic polypeptide
HVEGTFTSDVSSYLEEKAAKEFIAWLVKGRG

SEQ ID NO: 239 - protein, 31 aa, Artificial Sequence
  Description of Artificial Sequence Synthetic polypeptide
HVEGTFTSDVSSYLEEEAAKEFIAWLVKGRG

SEQ ID NO: 240 - protein, 31 aa, Artificial Sequence
  Description of Artificial Sequence Synthetic polypeptide
HVEGTFTSDVSSYLEEQAAKAFIAWLVKGRG

SEQ ID NO: 241 - protein, 31 aa, Artificial Sequence
  Description of Artificial Sequence Synthetic polypeptide
HVEGTFTSDVSSYLEGQAAKEFIAWLVGKRG

SEQ ID NO: 242 - protein, 31 aa, Artificial Sequence; identical to SEQ ID NO: 205
  Description of Artificial Sequence Synthetic polypeptide

SEQ ID NO: 243 - protein, 31 aa, Artificial Sequence; identical to SEQ ID NO: 206
  Description of Artificial Sequence Synthetic polypeptide

SEQ ID NO: 244 - protein, 32 aa, Artificial Sequence
  Description of Artificial Sequence Synthetic polypeptide
HXEGXXTSDXSSYLEXXXAXXFIAXLXXXXXX

SEQ ID NO: 245 - protein, 32 aa, Artificial Sequence
  Description of Artificial Sequence Synthetic polypeptide
HXEGTXTSDXSSYLEXXAAXEFIXWLVKXRXX

SEQ ID NO: 246 - protein, 32 aa, Artificial Sequence
  Description of Artificial Sequence Synthetic polypeptide
HXEGTFTSDVSSYLEXXAAKXFIXWLVKGRXX

SEQ ID NO: 247 - protein, 31 aa, Artificial Sequence
  Description of Artificial Sequence Synthetic polypeptide
XXEGTFTSDVSSYLEXQAAKEFIAWLVKGRG

SEQ ID NO: 248 - protein, 37 aa, Artificial Sequence
  Description of Artificial Sequence Synthetic polypeptide
XXNTATXATQRLXNFLXXXXXNXGXXLXXTXVGSNTY

SEQ ID NO: 249 - protein, 37 aa, Artificial Sequence [sequence omitted]

SEQ ID NO: 250 - protein, 37 aa, Artificial Sequence
  Description of Artificial Sequence Synthetic polypeptide
KCNTATCATQRLANFLIRSSNNLGAILSPTNVGSNTY

SEQ ID NO: 251 - protein, 37 aa, Artificial Sequence
  Description of Artificial Sequence Synthetic polypeptide
KCNTATCATQRLANFLVRTSNNLGAILSPTNVGSNTY

SEQ ID NO: 252 - protein, 37 aa, Artificial Sequence
  Description of Artificial Sequence Synthetic polypeptide
KCNTATCATQRLANFLVRSSNNLGPVLPPTNVGSNTY

SEQ ID NO: 253 - protein, 37 aa, Artificial Sequence
  Description of Artificial Sequence Synthetic polypeptide
KCNTATCATQRLANFLVHSNNNLGPVLSPTNVGSNTY

SEQ ID NO: 254 - protein, 37 aa, Artificial Sequence
  Description of Artificial Sequence Synthetic polypeptide
KCNTATCATQRLTNFLVRSSHNLGAALLPTDVGSNTY

SEQ ID NO: 255 - protein, 36 aa, Artificial Sequence
  Description of Artificial Sequence Synthetic polypeptide
CNTATCATQRLANFLVHSSNNFGAILSSTNVGSNTY

SEQ ID NO: 256 - protein, 37 aa, Artificial Sequence
  Description of Artificial Sequence Synthetic polypeptide
KCNTATCATQRLANFLVHSSNNFGAILPSTNVGSNTY

SEQ ID NO: 257 - protein, 37 aa, Artificial Sequence
  Description of Artificial Sequence Synthetic polypeptide
KCNTATCATQRLANFLVHSSNNFGPILPPTNVGSNTY

SEQ ID NO: 258 - protein, 37 aa, Artificial Sequence
  Description of Artificial Sequence Synthetic polypeptide
KCNTATCATQRLANFLVRSSNNFGPILPSTNVGSNTY

SEQ ID NO: 259 - protein, 36 aa, Artificial Sequence
  Description of Artificial Sequence Synthetic polypeptide
CNTATCATQRLANFLVHRSNNFGPILPSTNVGSNTY

SEQ ID NO: 260 - protein, 37 aa, Artificial Sequence
  Description of Artificial Sequence Synthetic polypeptide
KCNTATCATQRLANFLVHSSNNFGPVLPPTNVGSNTY

SEQ ID NO: 261 - protein, 37 aa, Artificial Sequence
  Description of Artificial Sequence Synthetic polypeptide
KCNTATCATQRLANFLVRSSNNFGPILPPTNVGSNTY

SEQ ID NO: 262 - protein, 36 aa, Artificial Sequence
  Description of Artificial Sequence Synthetic polypeptide
CNTATCATQRLANFLVRSSNNFGPILPPSNVGSNTY

SEQ ID NO: 263 - protein, 36 aa, Artificial Sequence
  Description of Artificial Sequence Synthetic polypeptide
CNTATCATQRLANFLVHSSNNFGPILPPSNVGSNTY

SEQ ID NO: 264 - protein, 37 aa, Artificial Sequence
  Description of Artificial Sequence Synthetic polypeptide
KCNTATCATQRLANFLVHSSNNLGPVLPPTNVGSNTY

SEQ ID NO: 265 - protein, 37 aa, Artificial Sequence
  Description of Artificial Sequence Synthetic polypeptide
KCNTATCATQRLANFLVHSSNNLGPVLPSTNVGSNTY

SEQ ID NO: 266 - protein, 36 aa, Artificial Sequence
  Description of Artificial Sequence Synthetic polypeptide
CNTATCATQRLANFLVHSSNNLGPVLPSTNVGSNTY

SEQ ID NO: 267 - protein, 37 aa, Artificial Sequence
  Description of Artificial Sequence Synthetic polypeptide
KCNTATCATQRLANFLVRSSNNLGPVLPSTNVGSNTY

SEQ ID NO: 268 - protein, 37 aa, Artificial Sequence
  Description of Artificial Sequence Synthetic polypeptide
KCNTATCATQRLANFLVRSSNNLGPILPPTNVGSNTY

SEQ ID NO: 269 - protein, 37 aa, Artificial Sequence
  Description of Artificial Sequence Synthetic polypeptide
KCNTATCATQRLANFLVRSSNNLGPILPSTNVGSNTY

SEQ ID NO: 270 - protein, 37 aa, Artificial Sequence
  Description of Artificial Sequence Synthetic polypeptide
KCNTATCATQRLANFLIHSSNNLGPILPPTNVGSNTY

SEQ ID NO: 271 - protein, 37 aa, Artificial Sequence
  Description of Artificial Sequence Synthetic polypeptide
KCNTATCATQRLANFLVISSNNFGPILPPTNVGSNTY

SEQ ID NO: 272 - protein, 36 aa, Artificial Sequence
  Description of Artificial Sequence Synthetic polypeptide
CNTATCATQRLANFLIHSSNNLGPILPPTNVGSNTY

SEQ ID NO: 273 - protein, 37 aa, Artificial Sequence
  Description of Artificial Sequence Synthetic polypeptide
KCNTATCATQRLANFLIRSSNNLGAILSSTNVGSNTY

SEQ ID NO: 274 - protein, 37 aa, Artificial Sequence
  Description of Artificial Sequence Synthetic polypeptide
KCNTATCATQRLANFLIRSSNNLGAVLSPTNVGSNTY

SEQ ID NO: 275 - protein, 37 aa, Artificial Sequence
  Description of Artificial Sequence Synthetic polypeptide
KCNTATCATQRLANFLIRSSNNLGPVLPPTNVGSNTY

SEQ ID NO: 276 - protein, 37 aa, Artificial Sequence
  Description of Artificial Sequence Synthetic polypeptide
KCNTATCATQRLTNFLVHSSHNLGAALLPTDVGSNTY

SEQ ID NO: 277 - protein, 37 aa, Artificial Sequence
  Description of Artificial Sequence Synthetic polypeptide
KCNTATCATQRLTNFLVHSSHNLGAALSPTDVGSNTY

SEQ ID NO: 278 - protein, 36 aa, Artificial Sequence
  Description of Artificial Sequence Synthetic polypeptide
CNTATCATQRLTNFLVHSSHNLGAVLPSTDVGSNTY

SEQ ID NO: 279 - protein, 37 aa, Artificial Sequence
  Description of Artificial Sequence Synthetic polypeptide
KCNTATCATQRLTNFLVRSSHNLGAALSPTDVGSNTY

SEQ ID NO: 280 - protein, 37 aa, Artificial Sequence
  Description of Artificial Sequence Synthetic polypeptide
KCNTATCATQRLTNFLVRSSHNLGAILPPTDVGSNTY

SEQ ID NO: 281 - protein, 37 aa, Artificial Sequence
  Description of Artificial Sequence Synthetic polypeptide
KCNTATCATQRLTNFLVRSSHNLGPALPPTDVGSNTY

SEQ ID NO: 282 - protein, 37 aa, Artificial Sequence
  Description of Artificial Sequence Synthetic polypeptide
KDNTATKATQRLANFLVHSSNNFGAILSSTNVGSNTY

SEQ ID NO: 283 - protein, 37 aa, Artificial Sequence
  Description of Artificial Sequence Synthetic polypeptide
ACNTATCATQRLANFLVHSSNNFGAILSSTNVGSNTY

SEQ ID NO: 284 - protein, 37 aa, Artificial Sequence
  Description of Artificial Sequence Synthetic polypeptide
SCNTATCATQRLANFLVHSSNNFGAILSSTNVGSNTY

SEQ ID NO: 285 - protein, 37 aa, Artificial Sequence
  Description of Artificial Sequence Synthetic polypeptide
KCNTATCATQRLANFLVHSSNNFGAILSPTNVGSNTY

SEQ ID NO: 286 - protein, 37 aa, Artificial Sequence
  Description of Artificial Sequence Synthetic polypeptide
KCNTATCATQRLANFLVHSSNNFGPILPSTNVGSNTY

SEQ ID NO: 287 - protein, 36 aa, Artificial Sequence
  Description of Artificial Sequence Synthetic polypeptide
CNTATCATQRLANFLVHSSNNFGPILPSTNVGSNTY

SEQ ID NO: 288 - protein, 36 aa, Artificial Sequence
  Description of Artificial Sequence Synthetic polypeptide
CNTATCATQRLANFLVHSSNNFGPVLPPSNVGSNTY

SEQ ID NO: 289 - protein, 37 aa, Artificial Sequence [sequence omitted]

SEQ ID NO: 290 - protein, 37 aa, Artificial Sequence; identical to SEQ ID NO: 257
  Description of Artificial Sequence Synthetic polypeptide

SEQ ID NO: 291 - protein, 32 aa, Artificial Sequence
  Description of Artificial Sequence Synthetic polypeptide
KCNTATCVLGRLSQELHRLQTYPRTNTGSNTY

SEQ ID NO: 292 - protein, 32 aa, Artificial Sequence
  Description of Artificial Sequence Synthetic polypeptide
CSNLSTCVLGRLSQELHRLQTYPRTNTGSNTY

SEQ ID NO: 293 - protein, 5 aa, Artificial Sequence
  Description of Artificial Sequence Synthetic peptide
GGXSS

SEQ ID NO: 294 - protein, 6 aa, Artificial Sequence
  Description of Artificial Sequence Synthetic peptide
GGXSSG

SEQ ID NO: 295 - protein, 7 aa, Artificial Sequence
  Description of Artificial Sequence Synthetic peptide
GGXSSGA

SEQ ID NO: 296 - protein, 8 aa, Artificial Sequence
  Description of Artificial Sequence Synthetic peptide
GGXSSGAX

SEQ ID NO: 297 - protein, 9 aa, Artificial Sequence
  Description of Artificial Sequence Synthetic peptide
GGXSSGAXX

SEQ ID NO: 298 - protein, 10 aa, Artificial Sequence
  Description of Artificial Sequence Synthetic peptide
GGXSSGAXXX

SEQ ID NO: 299 - protein, 11 aa, Artificial Sequence
  Description of Artificial Sequence Synthetic peptide
GGXSSGAXXXX